(19) United States
(12) Patent Application Publication
(10) Pub. No.: US 2017/0370934 A1
(43) Pub. Date: Dec. 28, 2017

(54) METHODS AND COMPOSITIONS FOR IMMUNOMODULATION

(71) Applicant: Nodality, Inc., South San Francisco, CA (US)

(72) Inventors: Andrew Hotson, Menlo Park, CA (US); Rachael Hawtin, San Carlos, CA (US); Andrew T. Conroy, Redwood City, CA (US); Erwan Le Scolan, San Francisco, CA (US)

(21) Appl. No.: 15/647,214

(22) Filed: Jul. 11, 2017

Related U.S. Application Data
(63) Continuation of application No. 14/525,013, filed on Oct. 27, 2014.
(60) Provisional application No. 62/057,977, filed on Sep. 30, 2014.
(60) Provisional application No. 61/949,867, filed on Mar. 7, 2014.
(60) Provisional application No. 61/915,245, filed on Dec. 12, 2013.
(60) Provisional application No. 61/895,816, filed on Oct. 25, 2013.

(51) Int. Cl. G01N 33/574; C07K 16/28; G01N 33/50; A61K 39/00

(52) U.S. Cl. CPC G01N 33/5743; G01N 33/505; C07K 16/2818; G01N 33/574; G01N 33/57415; A61K 2039/505; C07K 2317/21; C07K 2317/76; G01N 2333/70503; G01N 2333/70532

(57) ABSTRACT

The invention relates to immunomodulation of cells and the detection and use thereof, for example, in drug screening, including methods, compositions and systems therefor, or in an aspect of healthcare, such as prognosis, diagnosis, an aspect of treatment, monitoring, and the like, and methods, compositions, and systems therefor.

### CROSS-REFERENCE

This application is a continuation of U.S. patent application Ser. No. 14/525,013, filed Oct. 27, 2014, which claims the benefit of U.S. Provisional Patent Application No. 61/895,816, filed Oct. 25, 2013; U.S. Provisional Patent Application No. 61/915,245, filed Dec. 12, 2013; U.S. Provisional Patent Application No. 61/949,867, filed Mar. 7, 2014, and U.S. Provisional Patent Application No. 62/057,977, filed Sep. 30, 2014, each of which is incorporated by reference in its entirety.

### BACKGROUND OF THE INVENTION

In certain conditions, such as cancer, cellular pathways may be influenced by cells associated with the condition, e.g., tumor cells, such that certain cells, e.g., immune cells are inhibited or stimulated. Screening potential therapeutic agents, targeting therapy for the condition, and other activities may require an understanding of the functionality of pathways in one or more cell types, and/or optionally expression levels in the cell types and/or in diseased cells such as tumor cells of various ligands, receptors, or other cell components.

### SUMMARY OF THE INVENTION

The invention provides a method of treating a patient suffering from a pathological condition comprising treating the patient with a treatment for the condition. In some embodiments, an aspect of treating the patient with the treatment is based on an outcome of a treatment decision process. In some embodiments, the treatment decision process comprises consideration of at least two of a first, second, and/or third quantitative value, or a value or values derived from the at least two quantitative values. In some embodiments, the first, second, and third quantitative values are obtained from results of a first, second, and/or third assay, respectively. In some embodiments, the first assay comprises determining surface expression levels of a first immunomodulatory receptor (IMR) of a first cell population cell population (CP in a first sample from the patient. In some embodiments, the second assay comprises determining functional status of a second IMR in single cells of a second CP or a subpopulation thereof in a second sample from the patient. In some embodiments, the third assay comprises determining surface expression levels of an IMR ligand (IMRL) for a third IMR in a third cell population in a third sample from the patient. In some embodiments, the condition is cancer.

In some embodiments, the surface expression levels of the first IMR in the first assay are determined in single cells, or the surface expression levels of the IMRL of the third IMR in the third assay are determined in single cells, or both.

In some embodiments, the method of determination in the assay comprises cytometry. In some embodiments, the cytometry is flow cytometry or mass cytometry. In some embodiments, the cytometry is flow cytometry. In some embodiments, the cytometry is mass cytometry.

In some embodiments, the aspect of treating the patient comprises a decision to treat the patient or not treat the patient with the treatment, a choice of the treatment or a component of the treatment, a choice of the timing of the treatment or of a component of the treatment, a choice of a dosage of the treatment or a component of the treatment, or a combination thereof. In some embodiments, the outcome of the treatment decision process comprises a first likelihood of the patient responding to the treatment, a second likelihood of prolongation of the patient's life due to receiving the treatment, or a third likelihood of the patient experiencing an adverse treatment effect, or any combination of the first, second, and/or third likelihoods. In some embodiments, the assays comprise the first assay and the second assay, the assays are performed on single cells, the first and second samples are the same sample, the first and second IMRs are the same IMR, and the first and second cell populations are the same population, and the second quantitative value represents a functional status of the IMR for the subpopulation of the population, the process of obtaining the second quantitative value comprises gating the results for functional status of the IMR in the single cells of the cell population on the basis of the results of the determination of the expression level of the IMR in the same single cells of the population. In some embodiments, the gating comprises establishing a threshold for expression level of the IMR in a single cell and single cells in the cell population having an expression level of the IMR above the threshold are included in the subpopulation and single cells in the cell population having an expression level equal to or below, or below, the threshold are excluded from the subpopulation.

In some embodiments, the treatment is a combination treatment comprising an immunotherapy treatment. In some embodiments, the combination treatment further comprises a targeted treatment, a chemotherapy treatment, a radiation treatment, or a surgical treatment.

In some embodiments, the first and second cell populations are immune cell populations.

In some embodiments, the first and second immune cell populations are the same immune cell population. In some embodiments, the first and second immune cell populations are different immune cell populations. In some embodiments, the third cell population is a non-immune cell population. In some embodiments, the third cell population is a tumor cell population.

In some embodiments, the first sample and the second sample, and optionally the third sample, are the same sample. In some embodiments, the sample is a blood or blood-derived sample, or bone marrow or bone marrow-derived sample. In some embodiments, the first and second samples, and optionally the third sample, are solid samples or solid-sample-derived samples. In some embodiments, the sample comprise a tumor sample. In some embodiments, the tumor sample is a primary tumor sample or a metastatic tumor sample. In some embodiments, the first and second samples comprise tumor-infiltrating lymphocytes (TILS) derived from a solid tumor sample and the third sample comprises tumor cells derived from the same solid tumor sample. In some embodiments, the sample is a peripheral blood mononuclear cell (PBMC) sample.

In some embodiments, the surface expression levels of a plurality of IMRLs in the third assay are determined in single cells. In some embodiments, the plurality of IMRLs comprises a plurality of IMRLS of FIG. 15 and the description thereof. In some embodiments, the surface expression levels of a plurality of first IMRs in the first assay are determined. In some embodiments, the surface expression levels of a plurality of second IMRs in the second assay are determined. In some embodiments, the surface expression levels of a plurality of IMRs in the first assay and the second assay are determined. In some embodiments, the plurality of IMRs comprises a plurality of IMRs of FIG. 15 and the description thereof.

In some embodiments, the condition is cancer, the therapy is a combination therapy comprising immunotherapy, in first assay and second assay a plurality of IMRs is assayed, and the aspect of the treatment comprises choice of the combination therapy.

In some embodiments, the assay of the functional status of the IMR in the second assay comprises determining the change in an activation level of an intracellular activatable element or change in expression level of an intracellular expression element. In some embodiments, the activatable element is an activatable element of TABLE 1, or FIG. 20. In some embodiments, the activatable element comprises p-ERK or p-AKT.

In some embodiments, the treatment decision process further comprises consideration of a characteristic of the patient. In some embodiments, the characteristic comprises a genetic characteristic, age, gender, race, health status, previous treatment history, or any combination thereof. In some embodiments, the first and second cell populations comprise a first and second cell immune cell population of TABLE 1 or FIG. 17. In some embodiments, the first and second cell population are the same cell population. In some embodiments, the cell populations are identified by surface expression levels of at least three of the cell surface markers of Table 1 or FIG. 17.

In some embodiments, the IMRL corresponds to the first IMR in the first assay or the second IMR in the second assay. In some embodiments, the second assay comprises determining the functional status of the IMR in the presence and absence of an immunotherapeutic agent. In some embodiments, the second assay comprises determining the functional status of the IMR in the presence and absence of a plurality of immunotherapeutic agents.

The invention further provides a kit comprising (i) a distinguishably detectable binding element configured for use in binding to and distinguishably detecting a first intracellular element; and (ii) a distinguishably detectable binding element configured for use in binding to and distinguishably detecting a cell surface IMR on the cell or a cell surface IMRL on a cell of a population of cells of a non-immune cell type. In some embodiments, a change in the expression level and/or activation level of the first intracellular element in a cell of an immune cell type in response to exposure of the cell to an activator of the immune cell type is indicative of activation of the cell. In some embodiments, the kit further comprises the activator of the immune cell type. In some embodiments, the kit comprises a plurality of distinguishably detectable binding elements configured for use in binding to and distinguishably detecting a plurality of different cell surface IMRs or a plurality of different cell surface IMRLs. In some embodiments, the kit further comprises instructions for use of the kit in an assay for predicting the response of a patient to immunotherapy. In some embodiments, the immunotherapy is an immunotherapy that directly or indirectly affects activation of the population of cells of the immune cell type. In some embodiments, the kit further comprises a plurality of distinguishably detectable binding elements, each configured for use in binding to and distinguishably detecting a different cell surface marker. In some embodiments, the level of at least two of the plurality of different cell surface markers can be used to type the cell as a cell of an immune cell population. In some embodiments, the plurality of different surface IMRs and/or the plurality of different cell surface IMRLs is a plurality of different surface IMRs and/or a plurality of different cell surface IMRLs of FIG. 15 and the description thereof. In some embodiments, the plurality of IMRs comprise PD-1 and CTLA-4 and the plurality of IMRLs comprise at least two of B7-1, B7-2, PDL-1, and PDL-2. In some embodiments, the plurality of cell surface markers comprise a plurality of cell surface markers listed in TABLE 1 or FIG. 17. In some embodiments, the cell surface IMR or the cell surface IMRL comprises an IMR or an IMRL or of FIG. 15 and the description thereof. In some embodiments, the IMR is PD-1 and the IMRL is PDL-1 or PDL-2. In some embodiments, the intracellular element is an intracellular activatable element. In some embodiments, the activatable element is an activatable element of TABLE 1 or FIG. 20.

The invention further provides a kit comprising at least three distinguishably detectable binding elements. In some embodiments, the at least three distinguishably detectable binding elements are configured for use in binding to and distinguishably detecting at least one, two, or three different cell surface IMRs on single cells of an immune cell population and/or at least one, two, or three cell surface IMRLs on single cells of a non-immune cell population.

In some embodiments, the IMR or IMRs, and/or IMRL or IMRLs, are an IMR and/or IMRL of FIG. 15 and the description thereof. In some embodiments, the kit comprises at least five distinguishably detectable binding elements. In some embodiments, the at least five distinguishably detectable binding elements are configured for use in binding to and distinguishably detecting at least one, two, three, four, or five different cell surface IMRs on single cells of an immune cell population and/or at least one, two, three, four cell or five surface IMRLs on single cells of a non-immune cell population. In some embodiments, the kit comprises at least five detectable binding elements. In some embodiments, at least three distinguishably detectable binding elements are each configured for use in binding to and distinguishably detecting a cell surface IMR on the cell of an immune cell population or a cell surface IMRL on a cell of a non-immune cell population.

The invention further provides a pharmaceutical package comprising one or more immunotherapeutic agents and (i) instructions and/or an imprint indicating that the one or more immunotherapeutic agents is to be used for treatment of a patient who suffers from a pathological condition; (ii) instructions and/or an imprint indicating that the patient is to be stratified by one or more the methods described herein that produces a result that can be used to determine if condition (i)(a), (b), (c), and/or (d) is satisfied; and/or (iii) one or more necessary materials to carry out the one or more of methods of part (ii).

In some embodiments, cells (e.g., cells associated with the patient's pathological condition, an immune cell population from a sample from the patient) or non-cell samples (e.g., a non-cell liquid from a sample from the patient) can be characterized. In some embodiments, cells associated with the patient's pathological condition are characterized by surface expression of an IMRL at a level greater than, or greater than or equal to a threshold level of expression or surface expression of a plurality of different IMRLs at levels greater than, or greater than or equal to, a plurality of threshold expression levels. In some embodiments, an immune cell population from a sample from the patient is characterized by surface expression level of a first IMR that is greater than, or greater than or equal to a threshold expression level. In some embodiments, an immune cell population from a sample from the patient is characterized by a change in the expression level and/or activation level of an intracellular element that is less than, or less than or equal to a threshold change. For example, the change in the expression level or activation level of the intracellular element in a cell of an immune cell type is in response to contact with an activator of that immune cell type and is indicative of the activation level of the cell, and the change in the level may be measured in the presence and/or absence of an activator and/or inhibitor of an IMR that can be expressed on the cell of the immune cell type. In some embodiments, a non-cell liquid from a sample from the patient contains an immune effector molecule at a level greater than, greater than or equal to, less than, or less than or equal to a threshold level. In some embodiments, any combination of the above-mentioned cells or non-cell samples can be characterized. In some embodiments, the pharmaceutical package further comprises one or more components for use in gathering, treating, and/or transporting one or more samples from the patient for use in the one or more methods of the above-mentioned characterizations.

In some embodiments, the pathological condition is cancer. In some embodiments, the cells associated with the pathological condition comprise tumor cells. In some embodiments, the cancer is characterized by tumor cell surface expression of an IMRL that modulates an inhibitory IMR of FIG. 15 and the description thereof. In some embodiments, the tumor cell surface expression level of the IMRL is greater than, or greater than or equal to, a threshold level. In some embodiments, the cancer is characterized by tumor cell surface expression of an IMRL that activates PD-1. In some embodiments, the cancer is characterized by tumor cell surface expression of plurality of IMRLs, each of which modulates a different inhibitory IMR of FIG. 15 and the description thereof. In some embodiments, the surface expression level of each of the IMRLs is greater than, or greater than or equal to, a threshold level for surface expression for that IMRL. In some embodiments, the cancer is characterized by tumor cell surface expression of an IMRL that activates PD-1 and tumor cell surface expression of an IMRL that activates CTLA4.

In some embodiments, the intracellular element is an intracellular activatable element and the activation level of the element is indicative of the activation level of the cell. In some embodiments, the intracellular activatable element is an activatable element of TABLE 1 or FIG. 20. In some embodiments, the intracellular activatable element comprises p-ERK, p-AKT, p-ZAP70, PLCg, p-PKC θ, p-p38, or pNFkBp65. In some embodiments, the activatable element comprises p-ERK or p-AKT. In some embodiments, the intracellular activatable element comprises p-STAT1, p-STAT3, p-STAT4, p-STAT5, or p-STAT6, or a combination thereof.

The invention further provides a method for screening a first agent at a first screening level. The method comprises (i) contacting a first immune cell population expressing a first IMR on their surfaces with the first agent and activating the cells of the first population by contacting them with an activator; (ii) activating the cells of a second immune cell population expressing the first IMR on their surfaces that have not been contacted with the first agent by contacting them with the activator; (iii) determining (a) expression levels of an intracellular expression element in single cells of the first population or a subpopulation thereof and expression levels of the intracellular element in single cells of the second population or a subpopulation thereof, and/or (b) activation levels of an intracellular activatable element in single cells of the first population or a subpopulation thereof and activation levels of the intracellular activatable element in single cells of the second population or a subpopulation thereof; (iv) making a determination to send or not send the agent to a second screening level based on the results of (iii). In some embodiments, the intracellular expression element is an element whose expression levels changes upon activation of the cells of the first and second immune cell populations. In some embodiments, the intracellular activatable element is an activatable element whose activation level changes upon activation of the cell of the first and second immune cell populations. In some embodiments, the determination of step (iv) comprises an evaluation of a result of a comparison of the expression levels of the intracellular element and/or the activation levels of the intracellular activatable element in the single cells of the first population, or a first quantitative value derived therefrom, with the expression levels of the intracellular element and/or the activation levels of the intracellular activatable element in the single cells of the second population, or a second quantitative value derived therefrom. In some embodiments, the result is a third quantitative value. In some embodiments, the determination of step (iv) comprises comparing the third quantitative value with a threshold value to determine if the third value is greater than, greater than or equal to, less than, or less than or equal to the threshold value. In some embodiments, the agent is sent to the second screening level if the third quantitative value is greater than, or greater than or equal to, the threshold value. In some embodiments, the agent is sent to the second screening level if the third quantitative value is less than, or less than or equal to, the threshold value. In some embodiments, the first and second cell populations are the same immune cell population. In some embodiments, the identity of the first and second immune cell populations is determined by determining the levels of at least one cell surface marker in single cells of the first and second immune cell populations.

In some embodiments, the method further comprises determining the expression levels of the intracellular element and/or the activation levels of the intracellular activatable element in single cells of a third immune cell population type that have not been activated and that have not been contacted with the agent. In some embodiments, the first, second, and third immune cell populations are the same immune cell population.

In some embodiments, the method further comprises determining surface expression levels of the first IMR in single cells of the first and second immune cell populations. In some embodiments, the expression levels of the intracellular element and/or the activation levels of the intracellular activatable element are determined in subpopulations of the first and second immune cell populations. In some embodiments, a cell is gated into the subpopulation of the first or second population on the basis of its surface expression level of the first IMR. In some embodiments, a cell is gated by comparing its surface expression level of the IMR to a threshold expression level value for the first IMR. In some embodiments, the cell is gated into the subpopulation if its surface expression level of the first IMR is greater than the threshold value, or greater than or equal to the threshold value.

In some embodiments, the method further comprises screening a second agent in combination with the first agent and step (i) of the above-mentioned method of screening further comprises contacting the first immune cell population with the second agent; and the cells of the second immune cell population further express the second IMR on their surfaces and in step (ii) of the above-mentioned method of screening the cells of the second population have not been contacted with the second agent. In some embodiments, the second agent is different from the first agent. In some embodiments, the cells of the first immune cell population further express a second IMR on their surfaces. In some embodiments, the method further comprises determining surface expression levels of the second IMR in single cells of the first and second immune cell populations. In some embodiments, the expression levels of the intracellular expression element and/or the activation levels of the intracellular activatable element are determined in subpopulations of the first and second populations. In some embodiments, a cell is gated into the subpopulation of the first and second population on the basis of its surface expression level of the first IMR and its surface expression level of the second IMR. In some embodiments, a cell is gated by comparing its surface expression level of the first IMR to a threshold expression level value for the first IMR and its surface expression level of the second IMR to a threshold expression level value for the second IMR. For example, the cell is gated into the subpopulation if its surface expression level of the first IMR is greater than the threshold value for the surface expression level of the first IMR and its surface expression level of the second IMR is greater than the threshold value for the surface expression level of the second IMR, or greater than or equal to the threshold values for the surface expression of the first and second IMRs.

In some embodiments, the cells of the first and second immune cell populations expressing the first IMR have been induced to express the first IMR by activation of the cells of the first and second immune cell populations at a time previous to steps (i) and (ii). In some embodiments, the cells are derived from a sample from a healthy individual, a plurality of samples from the healthy individual, or a plurality of samples from a plurality of healthy individuals. In some embodiments, the cells are from cell lines. In some embodiments, the cells are derived from a sample from an individual suffering from a pathological condition, or a plurality of samples from the individual, or a plurality of samples from a plurality of individuals suffering from the pathological condition. In some embodiments, the pathological condition is cancer.

The invention further provides a method of determining a phenotype of a population of cells of an immune cell population in a sample from a patient suffering from a pathological condition, comprising determining in single cells of the immune cell population surface expression levels of at least at least three different IMRs, and determining the phenotype based on the levels of the at least three different IMRs. In some embodiments, the method comprises determining the phenotype based on surface expression levels of at least 4 different IMRs on single cells of the population. The invention further provides a method of treating a patient comprising determining an immunotherapy for the patient, based on a phenotype determined by the above-mentioned method. In some embodiments, the immunotherapy is a combination immunotherapy. In some embodiments, the combination immunotherapy is a combination comprising at least two different immunotherapies.

The invention further provides a method of determining a phenotype of a population of cells of an non-immune cell population in a sample from a patient suffering from a pathological condition, comprising determining in single cells of the cell population surface expression levels of at least at least three different IMRLs and determining the phenotype based on the levels of the at least three different IMRLs. In some embodiments, the method comprises determining the phenotype based on surface expression levels of at least 4 different IMRLs on single cells of the population. The invention further provides a method of treating a patient comprising determining an immunotherapy for the patient based on a phenotype determined by the above-mentioned method. In some embodiments, the immunotherapy is a combination immunotherapy. In some embodiments, the immunotherapy is a combination immunotherapy. In some embodiments, the combination immunotherapy is a combination of two different immunotherapies.

The invention further provides a method of determining a phenotype of a population of cells of an immune cell population in a sample from a patient suffering from a pathological condition, comprising determining in single cells of the cell population a functional status of an IMR expressed on the surface of the cells and determining the phenotype based on the functional status of the IMR. In some embodiments, the method comprises determining the phenotype based on surface expression levels of at least 2 different IMRs expressed on the surfaces of single cells of the population. In some embodiments, the method further comprises determining the surface expression levels of the IMR in the single cells. In some embodiments, the cell population is a subpopulation of an immune cell population. In some embodiments, each single cell is placed or not placed in the subpopulation based on its surface expression level of the IMR. The invention further provides a method of treating a patient comprising determining an immunotherapy for the patient based on a phenotype determined by the above-mentioned method. In some embodiments, the immunotherapy is a combination immunotherapy. In some embodiments, the combination immunotherapy is a combination of two different immunotherapies.

The invention further provides a method of treating a patient suffering from a pathological condition. The method comprises treating the patient with a treatment for the condition. In some embodiments, an aspect of treating the patient with the treatment is based on an outcome of a treatment decision process comprising consideration of a quantitative value, or a value or values derived from the quantitative value. In some embodiments, the quantitative value is obtained from results of an assay comprising determining functional status of an IMR in single cells of an immune cell population or a subpopulation thereof in a sample from the patient. In some embodiments, the method further comprises determining surface expression levels of the IMR in the single cells. In some embodiments, the method is performed using a subpopulation of the immune cell population. In some embodiments, single cells of the subpopulation are gated into the subpopulation on the basis of the surface expression level of the IMR of the single cell. In some embodiments, the method of determination in the assay comprises cytometry. In some embodiments, the cytometry is flow cytometry or mass cytometry. In some embodiments, the cytometry is flow cytometry. In some embodiments, the cytometry is mass cytometry. In some embodiments, the aspect of treating the patient comprises a decision to treat the patient or not treat the patient with the treatment, a choice of the treatment or a component of the treatment, a choice of the timing of the treatment or of a component of the treatment, a choice of a dosage of the treatment or a component of the treatment, or a combination thereof. In some embodiments, the outcome of the treatment decision process comprises a first likelihood of the patient responding to the treatment, a second likelihood of prolongation of the patient's life due to receiving the treatment, or a third likelihood of the patient experiencing an adverse treatment effect, or any combination of the first, second, and/or third likelihoods. In some embodiments, the pathological condition is cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an example of the immune system cell communication network.

FIG. 2 provides results from Example 10.

FIG. 3 shows patient demographics for Example 11.

FIG. 4 shows monocyte hyporesponsiveness in melanoma vs. healthy patients.

FIG. 5 shows TCR signaling in various patient groups and health individual in Example 11.

FIG. 6 shows reduced IL-15 signaling in samples from patients that received ipilimumab.

FIG. 7 shows CTLA-4 defined differential signaling populations in CD4+ T cells.

FIG. 8 shows ipilimumab promotes in vitro T cell activation.

FIG. 9 shows co-stimulation effects of anti-CD3, anti-CD28, anti-PD1, and anti-ICOS, in various combinations, in normal T cells.

FIG. 10 shows signaling pathways interrogated in healthy and CLL T cells.

FIG. 11 shows basal signaling in CLL vs. healthy T cells.

FIG. 12 shows modulated signaling in CLL vs. healthy T cells.

FIG. 13 shows increased TCR signaling in CLL PD1+ CD8 T cells compared to healthy.

FIG. 14 shows decreased TCR induced proliferation in CLL PD-1+ cells compared to healthy, c311s activated 48 hr with anti-CD3/CD28+/−PD-1 blockade.

FIG. 15 shows exemplary immunomodulator receptors (IMRs), which can be expressed on the surface of cells of one or more immune cell populations and which have a role in immunomodulation in normal immune function as well as immunomodulation in a variety of pathological conditions, for example, immunosuppression in cancer, and which can be inhibitory (decrease the activation of the cells in response to one or more activators) or costimulatory (increase activation of the cells in response to one or more activators). A single cell may have multiple IMRs, which can be of either or both types (inhibitory and/or costimulatory) The inhibitory IMRs shown in this Figure, and their corresponding IMRLs, as well as an additional inhibitory IMR not shown in the Figure, and its IMRL, are
inhib IMR: CTLA-4, IMRLs: B7-1 (aka CD80), B7-2 (aka CD86)
inhib IMR: PD-1, IMRLs: PD-L1 (aka CD274, B741), PDL-2 (aka CD273, B7DC)
inhib IMR: BTLA (aka CD272), IMRL: HVEM
inhib IMR: LAG3 (aka CD223), IMRL: MHC class II molecules
inhib IMR: TIM-3 (HAvcr2), IMRL: Gal9
inhib IMR: VISTA, IMRL: unknown (putatively VISTAL, or the ligand for VISTA);
not shown in FIG. 15: inhib IMR: A2aR, IMRL: adenosine. Treg cells express high levels of the exoenzymes CD39 (aka NTPDase 1), which converts extracellular ATP to AMP, and CD73 (aka 5′-NT), which converts AMP to adenosine. Given that A2aR engagement by adenosine drives T cells to become Treg cells, this can produce a self-amplifying loop within the tumor, and expression levels of one or both. Thus A2aR is an important IMR, adenosine an important IMRL, and the exoenzymes CD39 and CD73 important immune effector molecules, any of which or any combination of which may be used as markers to determine immunosuppression, e.g., in the tumor microenvironment or in peripheral blood, and/or as target or targets for immunotherapy
The costimulatory IMRs and their corresponding IMRLs shown in the Figure, as well as an additional costimulatory IMR not shown in the Figure, and its IMRL are:
costim IMR: CD28, IMRL: B7-1 (aka CD80);
costim IMR: GITR (aka TNFRSR18, AITR, CD357, GITR-D), IMRL: GITRL;
costim IMR: OX-40 (aka CD134, TNFRSF4, ACT35, IMD16, TXGP1L), IMRL: OX-40L,
costim IMR: 4-1BB (aka CD137, TNFRSF9, ILA, CDw137), IMRL: 4-1BBL,
costim IMR: CD40L (aka CD 154, CD40 ligand; it is a costimulatory IMR despite the misleading name), IMRL: CD40,
costim IMR: CD27, IMRL: CD70
not shown in FIG. 15: costim IMR: ICOS (aka CD278), IMRL: B7-RP1 (aka CD275, ICOSLG).
FIG. 15 also shows a group of IMRs designated KIRs (killer cell immunoglobulin-like receptors), some of which are costimulatory IMRs and some of which are inhibitory IMRs, expressed on NK cells and certain T cells, IMRLs: MHC class I molecules

FIG. 16 shows selected results from Example 15, in which cells from samples from AML patients and from healthy volunteers were compared for surface expression levels of four costimulatory IMRs (4-1BB, OX-40, CD27, and GITR), three inhibitory IMRs (PD-1, LAG3, and TIM-3), and an IMRL (PD-L1), which were measured in single cells from different cell populations to determine surface expression levels of the IMRs and IMRLs in the different cell populations. Cell surface expression levels of PD-1, PD-L1, TIM-3, 4-1BB, and OX-40 in both CD4+ and CD8+ cell populations are shown; both PD-1 and OX-40 showed upregulation in cell populations in the AML patients.

FIG. 17 shows several populations of immune cells that can be the subject of the methods and compositions of the invention, and a selection of their corresponding cell surface markers. For a more complete list of cell surface markers, immune cell populations, and immune cell subpopulations, see TABLE 1. CD3+CD4+ cells are Thelper lineage cell populations, CD3+CD8+ cells are Tcytotoxic lineage cell populations, EM: effector memory cell subpopulations (T helper subpopulation if CD4+CD62Llow,CD45RAlow, Tcyto subpopulation if CD8+CD62LlowCD45RAhigh); CM: central memory cell subpopulation (T helper subpopulation if CD4+CD62LhighCD45RAlow, Tcyto subpopulation if CD8+CD62LhighCD45RAlow); E: effector cell subpopulation (Thelper subpopulation if CD4+CD62LlowCD45RAhigh, Tcyto subpopulation if CD8+CD62LlowCD45RAhigh); N: naïve cell subpopulation (Thelper subpopulation if CD4+CD62LhighCD45RAhigh, Tcyto subpopulation if CD8+CD62LhighCD45RAhigh). Further subpopulations of Tcells, from the Thelper subpopulation lineage, are Treg subpopulation if CD4+Foxp3+ which can be further subdivided into CD4+CD25+Foxp3+ Treg subpopulation and CD4+CD25-Foxp3+Treg subpopulation. CD3− cells are non-T cell lineage cell populations, of which B cells, NK cell, and monocytes are subpopulations. or Cell surface markers for B cell subpopulations are CD3−CD14−CD20+, which can be further divided into subpopulations on the basis of CD27 (+ or −), IgD (+ or −) (not shown, see Table 1. NK cell subpopulation is CD3−CD19−CD14−CD20−CD56+, further subdivided into subpopulations CD56bright and CD56dim.

FIG. 18 shows the protocol and results of Example 16.

FIG. 19 shows the protocol and results of Example 17.

FIG. 20 shows exemplary pathways interrogated by SCNP, activators for the pathways, intracellular expressed elements in the pathways, and intracellular activatable elements in the pathways.

### DETAILED DESCRIPTION OF THE INVENTION

The present invention incorporates information disclosed in other applications and texts. The following patent and other publications are hereby incorporated by reference in their entireties: Haskell et al, Cancer Treatment, 5th Ed., W.B. Saunders and Co., 2001; Alberts et al., The Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.; and Sambrook, Fritsche and Maniatis. “Molecular Cloning A laboratory Manual” 3rd Ed. Cold Spring Harbor Press (2001), all of which are herein incorporated in their entirety by reference for all purposes.

Also, patents and applications that are incorporated by reference include U.S. Pat. Nos. 7,381,535, 7,393,656, 7,563,584, 7,695,924, 7,695,926, 7,939,278, 8,148,094, 8,187,885, 8,198,037, 8,206,939, 8,214,157, 8,227,202, 8,242,248; U.S. patent application Ser. Nos. 11/338,957, 11/655,789, 12/061,565, 12/125,759, 12/125,763, 12/229,476, 12/432,239, 12/432,720, 12/471,158, 12/501,274, 12/501,295, 12/538,643, 12/551,333, 12/581,536, 12/606,869, 12/617,438, 12/687,873, 12/688,851, 12/703,741, 12/713,165, 12/730,170, 12/778,847, 12/784,478, 12/877,998, 12/910,769, 13/082,306, 13/091,971, 13/094,731, 13/094,735, 13/094,737, 13/098,902, 13/098,923, 13/098,932, 13/098,939, 13/384,181; International Applications Nos. PCT/US2011/001565, PCT/US2011/065675, PCT/US2011/026117, PCT/US2011/029845, PCT/US2011/048332; and U.S. Provisional Applications Ser. Nos. 60/304,434, 60/310,141, 60/646,757, 60/787,908, 60/957,160, 61/048,657, 61/048,886, 61/048,920, 61/055,362, 61/079,537, 61/079,551, 61/079,579, 61/079,766, 61/085,789, 61/087,555, 61/104,666, 61/106,462, 61/108,803, 61/113,823, 61/120,320, 61/144,68, 61/144,955, 61/146,276, 61/151,387, 61/153,627, 61/155,373, 61/156,754, 61/157,900, 61/162,598, 61/162,673, 61/170,348, 61/176,420, 61/177,935, 61/181,211, 61/182,518, 61/182,638, 61/186,619, 61/216,825, 61/218,718, 61/226,878, 61/236,281, 61/240,193, 61/240,613, 61/241,773, 61/245,000, 61/254,131, 61/263,281, 61/265,585, 61/265,743, 61/306,665, 61/306,872, 61/307,829, 61/317,187, 61/327,347, 61/350,864, 61/353,155, 61/373,199, 61/374,613, 61/381,067, 61/382,793, 61/423,918, 61/436,534, 61/440,523, 61/469,812, 61/499,127, 61/515,660, 61/521,221, 61/542,910, 61/557,831, 61/558,343, 61/565,391, 61/565,929, 61/565,935, 61/591,122, 61/640,794, 61/658,092, 61/664,426, and 61/693,429.

### Definitions

A “cell population,” as that term is used herein, encompasses a population of cells in which the majority of cells is of a same cell type or has a same characteristic. One convenient way to class single cells as part of a cell population is to determine the level of a cell surface marker of a given cell population on the single cell. The term “cell surface marker” and “extracellular cell marker” are used interchangeably herein. For example, T cells can be identified and classed based on the presence or absence, or relative abundance, of the CD4+ marker; thus one cell population can be CD4+ T cells, or T helper cells. Such markers and classifications are well-known in the art and any suitable method of classification may be used. Some exemplary cell surface markers and cell populations are shown in Table 1. A cell population can also be a subpopulation of another cell population. For example, the Thelper cell population is a subpopulation of the T cell lineage population, and the Thelper effector population is a subpopulation of the Thelper population. Other examples of cell populations that are subpopulations of another cell population are shown in Table 1.

An “immune cell population,” as that term is used herein, encompasses populations of cells of the immune system, for example, the human immune system. Such cell populations are known in the art and any suitable system for classifying cells as cells of an immune cell population may be used.

A “non-immune cell population,” as that term is used herein, encompasses any population of cells that is not an immune cell population, for example, any population of human cells, whether normal or abnormal, that is not an immune cell population, such as a population of cancer cells. In some pathological conditions, a population of immune cells may also be an abnormal, e.g., pathological population, such as a cancer cell population. For example, in certain hematological malignancies, such as AML, immune system cells themselves may be cancer cells. In these cases, the cells are classed as part of a non-immune population, for example, a tumor population, despite their origin as immune cells.

Other meanings are as in the description below.

### Description

In many pathological conditions, modulation of the immune response to the condition by cells associated with the pathological condition is an important aspect of the condition. One such pathological condition is cancer, where the tumor cells themselves develop strategies of immunosuppression to decrease various aspects of the host immune response; modulation of the immune system occurs in other pathological conditions as well, e.g., autoimmunity and HIV infection, and the invention encompasses such conditions. However, for convenience, the invention is described in many instances herein in terms of cancer; one of skill in the art will make the necessary alterations for any particular pathological condition. Detailed descriptions of immunosuppression in cancer and cancer immunotherapies are available, see Characiejus et al. Anticancer Res 31:639-648 (2011); Disis, M., Cancer Immunol. Immunother. 60:433-442 (2011 Kirkwood et al., CA Cancer J. Clin. 62:309-335 (2012); Pardoll, D. M., Nature Reviews, Cancer 12:252-264 (2012); Spranger and Gajewski, J. for Immunotherapy of Cancer 1:16-30 (2013); Vanneman, M., and Dranoff, G., Nature Reviews: Cancer 12:237-251 (2012); all of which are hereby incorporated by reference herein in their entireties.

One aspect of immunomodulation, e.g., immunosuppression, and of some of the methods and compositions of the invention involves immunomodulatory receptors (IMRs) on immune cells, and their interactions with IMR ligands (IMRLs) normally expressed on certain immune cell populations, e.g., antigen-presenting cells (APCs), and other cell populations, e.g., MHC I molecules, some or all of which can also be expressed on the surfaces of cells associated with a pathological condition, e.g. cancer cells. In certain cases, e.g., A2aR, the IMRL is a soluble factor (e.g., adenosine) and the pathological condition can affect its levels in the extracellular environment. Immune cells, e.g., T cells and non-T cells such as NK cells, monocytes, B cells, and dendritic cells (DC), and subpopulations thereof, such as Treg and Tcyto, express a variety of receptors that either inhibit the activation of the immune cell, (e.g., in the T cell, stimulation at the T Cell Receptor (TCR), similarly with other receptor or receptors for a particular immune cell population, see Table 1), or activate (costimulate) the activation of the cells. Both inhibitory and activating (costimulatory) receptors are referred to as “immunomodulatory receptors” (IMRs) herein. Tumor cells, as well as antigen-presenting cells (APCs) and other cells, often express IMR ligands (IMRLs) on their surface that interact with one or more of these receptors, thus blunting the immune response and decreasing effectiveness of the immune system in eradicating the tumor. In certain cases, such as the A2aR IMR, the ligand is a soluble molecule, e.g., adenosine. See, e.g., FIG. 15, which shows various stimulatory (costimulatory) and inhibitory IMRs found on T cells and the corresponding ligands found on, e.g., APCs or tumor cells, and Table 1, which provides exemplary methods of activating various immune cell populations. The description of FIG. 15 in the Brief Description of the Drawings provides further details and description, as well as additional IMRs and IMRLs that are encompassed by the methods and compositions of the invention.

Current therapies in cancer include immunotherapies, in which this blunting of the immune system is partially or completely reversed; various strategies may be employed, alone or, in many cases preferably, in combination. As used herein, an “immunotherapy” encompasses any therapy directed at altering modulation of the immune system of a patient, where the patient's immune system has been modulated by the pathological condition from which he or she suffers, e.g., immunotherapies in cancer seek to counteract, by one or preferably more than one, mechanism the immune suppression seen in a particular cancer. Immunotherapy, e.g., cancer immunotherapy, may be directed at any aspect of immunosuppression, or multiple aspects, for example, at modulating one or more of IMR-IMRL interactions, such as immune checkpoint blockade (blocking an inhibitory IMR with an antagonist, or blocking an inhibitory IMRL) but also including activating a costimulatory IMR); vaccination to bolster the immune response (often used in combination with modulation of IMR-IMRL interaction and generally involving DCs); cytokine therapy, e.g., treating a patient with IL-2, to bolster immune response; and adoptive immunotherapy, e.g., treating a patient with T cells that have been removed from the patient and modulated ex vivo to increase their tumor-killing capacity. Other immunotherapies are known and any immunotherapy described in, e.g., Characiejus et al. Anticancer Res 31:639-648 (2011); Disis, M., Cancer Immunol. Immunother. 60:433-442 (2011); Kirkwood et al., CA Cancer J. Clin. 62:309-335 (2012); Pardoll, D. M., Nature Reviews, Cancer 12:252-264 (2012); Spranger and Gajewski, J. for Immunotherapy of Cancer 1:16-30 (2013); Vanneman, M., and Dranoff, G., Nature Reviews: Cancer 12:237-251 (2012); all of which are hereby incorporated by reference herein in their entireties, may be an immunotherapy or provide an immunotherapeutic agent for the methods and compositions described herein, except, as used herein, “immunotherapy” does not encompass therapies in which an antibody targeting a tumor-associated antigen (TAA) is used, alone or conjugated to a therapeutic agent, to directly attack the tumor, even though it involves a component of the immune system, an antibody or a fragment thereof; such therapies are a type of “targeted therapies,” see, e.g., Vanneman, M., and Dranoff, G., Nature Reviews: Cancer 12:237-251 (2012).

Thus, one such strategy, or immunotherapy, is to modulate the activation of an IMR by its corresponding IMRL or IMRLs; this type of therapy is sometimes called checkpoint therapy if the therapy is aimed at decreasing the interaction between an inhibitory IMR and its ligand or ligands; however, therapies in which costimulatory IMRs are activated are also being developed. The therapy may be aimed at blocking the IMR if it is an inhibitory IMR, or blocking one or more ligands for the inhibitory IMR, or activating a costimulatory IMR with an agonist to its IMRL, or otherwise modulating the IMR-IMRL interaction, and/or modulating the activity of the IMR, so that the activity of the IMR and its IMR pathway is modulated-increased, in the case of a costimulatory IMR, or decreased, in the case of an inhibitory IMR. The ultimate result is thought to be that cells of one or more immune cell populations experience less immunosuppression and ultimately the immune system is able to attack and destroy the tumor cells. A well-known example of such therapy is ipilimumab therapy for malignant melanoma, which blocks the CTLA-4 (inhibitory) receptor, thus stimulating immune cells, e.g., T cells. Other similar therapies are being developed or tested, such as molecules to block the PD-1 (inhibitory) IMR or one or both of its cognate ligands. Anti-PD-1 therapies are now being tested, and some response is being seen in non-small cell lung cancer (NSCLC) and in renal cancer, but not all patients respond, and current stratification techniques are not effective. For example, patient stratification may be attempted by analyzing the level of expression of an IMRL, e.g., PD1 ligand (PD-L1) on tumor cells. Every IMR on T cells or other immune cells represents both a possible avenue for tumor cells to affect the immune system, however, and analysis of a single IMR alone may not prove sufficient for prediction of response to therapy. Multiple IMRs also present multiple potential targets for immunotherapy; thus, as mentioned, in some cases, immunotherapy may be used to inhibit an inhibitory IMR pathway or pathways, or stimulate activating (costimulatory) IMR pathway or pathways. Diagnosis, prognosis, monitoring, selection of an aspect of treatment of a patient, and screening candidate agents, e.g., drug candidates, to develop for such therapies are all aided by understanding and using knowledge of the pathways involved in the activity of IMRs (IMR pathways), especially at the single cell level, because the initial effect of an IMR pathway is at the level of the cell expressing the IMR on its surface.

Thus, methods, such as selection of an aspect of therapy for a patient suffering from a particular condition, e.g., cancer may be based at least in part on characterization of one or more of the IMR pathways, for example, the functional status of one or more IMR pathways.

The advantage of using functional status over traditional biomarkers such as expression levels, is that it gives a measure of the actual state of single cells of a cell population, e.g., a cell population from a sample from an individual such as a patient, or a cell population used in screening drug candidates. Unlike most traditional biomarkers, functional status relies on modulating the IMR or IMRs, preferably in single cells, to determine the level at which the particular IMR or IMRs is functioning, e.g., by activating the IMR or IMRs. When practiced with single cells, the functional status of all the cells of a population may be analyzed, one by one. By directly interrogating the functional status of an IMR/IMR pathway, one bases decisions on how the cells actually respond to a stimulus, generally a stimulus that results in activation similar to activation in the body, and how that response is modified by the one or more IMRs. Although other values, such as the expression level of the IMR, may also give an indication of, e.g., whether or not, and to what degree, the IMR is affecting a particular cell, it is not a direct indication, and does not take into account the complexity of the cell's actual function. For example, a particular cell may express a particular IMR at a high level, but, when the IMR is activated in conjunction with overall activation of the cell, the IMR may have little or no effect on the cell's response to overall activation. In this case, just using the expression level of the IMR as a marker for its effect in the cell will give an erroneous view of its influence on the cell; by surface expression level, its effect should be high, but by actual interrogation of its effect, it is low. Though this is not necessarily true for any given cell, and in many cases expression levels may be sufficient to provide a marker for cell or cell population function, and/or may be useful in gating cells from a population so that the functional status of an IMR is determined only in cells expressing the IMR above a certain threshold level on their surface is determined, determining functional status eliminates many potential sources of inaccuracy.

As used herein, “functional status,” for example used in reference to an IMR pathway or IMR, encompasses the magnitude of effect or potential effect on (e.g., modulation of, or potential modulation of), the immune activity of a particular cell or cell population due to the effects or potential effects of the IMR/IMR pathway in that cell or cell population. For example, if, when an immune cell is activated in the presence of activation of an IMR/IMR pathway, and in the absence of such activation, there is no difference in the activation level of the immune cell, the functional status of the IMR/IMR pathway is low in that cell, for example, can be expressed as 0. If, when an immune cell is activated in the presence of activation of an IMR/IMR pathway, and in the absence of such activation, there is a large difference in the activation level of the immune cell, the functional status of the IMR/IMR pathway is high. The functional status of an IMR/IMR pathway can be expressed in any suitable manner, for example as a quantitative value whose magnitude corresponds with magnitude of the effect of the IMR/IMRL on a particular cell or cell population. Depending on the IMR, and in some cases depending on the cell or cell population, the modulation or potential modulation of the activity of the cell or cell population can be an increase in the immune activity of the cell or cell population or a decrease in the immune activity of the cell or cell population.

The activation of the cell or population refers, e.g., to its response when activated by one or more activators for that particular cell or cell population, thus the functional status of an IMR or IMR pathway is generally, assessed in the context of activation of the cell or cell population in which it operates. For example, in T cells, activation can be achieved by well-known methods, such as those described herein and known in the art, e.g., to specifically activate T cells, one may contact the T cells with one or more activators of T cells, such as αCD3 and αCD28. The functional status of one or more IMRs/IMR pathways in T cells can be assessed by activating the T cells in the presence and absence of activation of the one or more IMRs/IMR pathways and assessing the difference in the activation of the T cells with and without such activation of the IMR/IMR pathway. Activation of the cell or cell population may be determined by any suitable means. In certain embodiments, determining the activation of a cell or cell population can include determining a change in the expression level of one or more intracellular expressed elements, and/or the change in the activation level of one or more intracellular activatable elements, as described herein, compared to the level without activation of the cell or cell population.

Intracellular activatable elements and intracellular expressed elements (also referred to herein as intracellular expression elements) are collectively referred to herein as “intracellular elements.” Intracellular expressed elements are typically proteins, e.g., intracellular proteins whose expression levels change in response to activation of the cell or cell population, e.g., where the change in expression levels corresponds to the level of activation of the cell or cell population. Intracellular activatable elements are typically proteins, e.g., proteins whose activation levels change in response to the activation of the cell or cell population, e.g., where the change in activation level corresponds to the level of activation of the cell or cell population. The kinetics of change in activation levels of one or more intracellular activatable elements, and/or expression levels of one or more intracellular expressed elements, can also be indicative of the activation of the cell or cell population. See Table 1 for examples of immune cell populations, cell surface markers, in vitro activators, intracellular activatable elements, and intracellular expressed elements.

TABLE 1
Immune cell population cell surface markers, in vitro activator(s), intracellular activatable elements, and
intracellular expressed elements

| | Exemplary | | | Intracellular | | | |
|---|---|---|---|---|---|---|---|
| | cell surface | | | activatable | Exemplary | Intracellular | Exemplary |
| | markers for | In vitro | | element² | intracellular | expressed | expressed |
| Immune cell | gating | activator | Exemplary | readout, | activatable | element³, | intracellular |
| population | population¹ | type | activators | type | elements | type | elements |
| T cell lineage | | | | | | | |
| T lineage | CD3+, | TCR | αCD3, | TCR | p-ERK, p- | Cytokine⁵ | TNFα, |
| | CD14− | activator⁴ | αCD28 | pathways | AKT, p- | | IFNγ, IL-2 |
| T helper⁶ | CD4+ | | | | ZAP70, | | IL-17 as |
| T helper | CD62Llow | | | | PLCγ, p- | | well for |
| effector | (or CD27), | | | | PKCθ, p- | | Thelper |
| memory | CD45RAhigh | | | | p38, | | |
| | CD45RAlow | | | | pNFκBp65, | | |
| T helper | CD62Lhigh | | | | p-IκB | | |
| central | (orCD27), | | | | | | |
| memory | CD45RAlow | | | | | | |
| T helper | CD62Llow | | | | | | |
| effector | (or CD27), | | | | | | |
| | CD45RAhigh | | | | | | |
| T helper | CD62Lhigh | | | | | | |
| naive | (orCD27), | | | | | | |
| | CD45RAhigh | | | | | | |
| T cyto | CD8+ | | | | | | |
| T cyto | CD62Llow | | | | | | |
| effector | (or CD27), | | | | | | |
| memory | CD45RAlow | | | | | | |
| T cyto | CD62Lhigh | | | | | | |
| central | (orCD27), | | | | | | |
| memory | CD45RAlow | | | | | | |
| T cyto | CD62Llow | | | | | | |
| effector | (or CD27), | | | | | | |
| | CD45RAhigh | | | | | | |
| T cyto | CD62Lhigh | | | | | | |
| naive | (orCD27), | | | | | | |
| | CD45RAhigh | | | | | | |
| Treg⁶ | CD4+Foxp3+ | | | | | | |
| Treg | CD25+, CD25− | | | | | | |
| CD25+ | | | | | | | |
| or − | | | | | | | |
| Treg | Helios+, | | | | | | |
| Helios | Helios− | | | | | | |
| Non-T | CD3− | | | | | | |
| lineage | | | | | | | |
| B cell | CD14−, | BCR | αIGM, | BCR | IkBα, p- | cytokine | IL-2, IL-4, |
| | CD20+ | activator⁷ | αIgG, | pathway | ERK, p- | | TNFα, IL-6, |
| Naïve B | CD27− | | αIgD, | | AKT | | IFNγ, IL-10, |
| cell | | | CD40L | | | | IL-12 |
| CD27+ B | CD27+ | TLR | TLR7, 8 & 9 | NFkB, | pSTAT (1, | | |
| cell | | agonist or | agonists | PI3K, | 3, 5, 6), p- | | |
| CD27+ | IgD+ | TLR | and/or | JAK/STAT | ERK, p- | | |
| Memory | | ligand | ligands | MAPK pathways | AKT, others | | |
| B cell | | (TLRL) | | | as in | | |
| | | | | | Detailed | | |
| | | | | | Description | | |
| | | | | | for pathways | | |
| NK cell | CD19−, | TLR | TLR2, 3, 4, | MAPK, | | Cytokine | IFNγ, GM- |
| | CD14−, | agonist, | 7, 8, 9, | PI3k, | | | CSF |
| | CD20−, | TLR | agonists | JAK/STAT, | | | |
| | CD56+ | TLRL | and/or | NFkB | | | |
| CD56dim | CD56dim | | ligands | pathways | | | |
| NK cells | | Fc | IgG, aCD16 | | | | |
| CD56bright | CD56bright | receptorγ | | | | | |
| NK cells | | ligand or | | | | | |
| | | agonist | | | | | |
| | | KIR | αCD158d | | | | |
| | | ligand or | | | | | |
| | | agonist or | | | | | |
| | | antagonist | | | | | |
| Non-T | CD3− | | | | | | |
| lineage | | | | | | | |
| Monocyte | CD20− | TLR | TLR1, 2, 3, | MAPK, | pSTAT (1, | Cytokine | TNFα, IL-6, |
| | CD14+ | agonist, | 4, 7, 8, 9 | PI3k, | 3, 5, 6), p- | | IFNγ |
| | | ligand | agonists | JAK/STAT, | ERK, p- | | |
| | | | and/or | NKkB | AKT, others | | |
| | | | ligands | pathways | as in | | |
| | | | | | Detailed | | |
| | | | | | Description | | |
| | | | | | for pathways | | |
| | | Fc | IgG | | | | |
| | | receptorγ | | | | | |
| | | ligand or | | | | | |
| | | agonist | | | | | |
| Dendritic Cell | CD20−, | TLR | TLR1, 2, 3, | MAPK, | pSTAT (1, | | |
| (DC) | CD19−, | agonist, | 4 7, 8, 9 | PI3k, | 3, 5, 6), | | |
| | CD14−, | ligand | agonists | JAK/STAT, | pERK, p- | | |
| | HLA-DR+ | | and/or | NFkB | AKT, others | | |
| Plasmacytoid | CD123+, | | ligands | pathways | as in | | |
| DC | CD11c− | | | | Detailed | | |
| (pDC) | | | | | Description | | |
| Myeloid | CD123− | | | | for pathways | | |
| DC (mDC) | CD11c+ | | | | | | |

¹Can be used to gate cells and to establish cell numbers in various subsets at various times before and after activation, and ratios thereof
²Change in activation level (activation level in cells exposed to activator compared to basal activation level measured in cells not exposed to activator) is detectable within minutes of activation and corresponds to level of activation of the cell in response to the activator. Changes in activation level can also be measured in longer time frames, e.g., hours, or days, such as at least 4, 8, 12, 16, 20, or 24 hours after contacting the cells with the activator, allowing intercellular communication events to occur.
³Change in expression level of intracellular expression element (expression level in cells exposed to activator compared to basal expression level measured in cells not exposed to activator) detectable in hours or days, e.g., at least 12, 16, 20, 24, 36 or 48 hours after contact with activator and can correspond to later events in activation. When cells from a variety of populations are in contact (e.g., in PBMC samples, or TILS from a solid tumor sample) for a prolonged period after contact with an activator, intercellular communication can play a role in a manner similar to intercellular communication in vivo, allowing a different view of overall interactions that occur in vivo, that may be different from the short-term activation events seen with activatable elements, which occur in a time frame (minutes), in which the intracellular and extracellular events necessary for cell-cell communication have not yet occurred.
Intracellular expression elements useful in the invention also include non-cytokine elements, such as cell cycle elements, e.g., Ki67 and Cyclin A2
⁴In certain embodiments, TCR activators are “specific T cell activators,” as they mainly or exclusively activate T cells, and TLR activators are “nonspecific T cell activators. Additionally, or alternatively, a in certain embodiments a “surrogate activator” may be used to determine the functional status of one or more IMRs in a cell population, e.g., T cells, e.g., use of cytokine activator (surrogate activator) such as IL-6, IL-10, IL-15, IL-21, IL-2, IL-4, IL-12, IFNa, or IFNg, or any combination thereof, and measuring the activation level of an activatable element in the JAK/STAT pathway, such as p-STATs (e.g., 1, 3, 4, 5, or 6 or any combination thereof), with or without modulation of the IMR or IMRs of interest, in single cells of the population, e.g., T cell population. See Example 14. In certain embodiments, basal levels of the activatable element or element may alone be a surrogate for functional status of an IMR or IMRs, e.g., by comparison with a value derived from analysis of samples with known functional status of the IMR or IMRs. See Example 14
⁵As used herein, the term “cytokines” includes the subclass of cytokines known as chemokines.
⁶For a subpopulation of a population, or a subpopulation of a subpopulation, only additional cell surface markers are shown
⁷In certain embodiments, BCR activators are “specific B cell activators,” as they activate mainly or exclusively B cells, and TLRs are “nonspecific B cell activators,” as they activate other cell populations besides B cells

Thus, the success of diagnosis, prognosis, and/or therapy, or even drug development, may depend on knowledge of the complex interplay between conditions produced by a pathological condition, such as tumor cells produced in a cancer, and one or more IMRs, in one or more immune cell population; in addition, or alternatively, knowledge of other aspects of immunosuppression may be required or useful. Certain embodiments of the current invention are based on characterization of one or more of IMR pathways, in one or more immune cell populations, to, e.g., guide diagnosis, prognosis, monitoring, and/or therapy for pathological conditions, such as cancer, and/or to aid in drug development for these conditions. The characterization may include characterizing the surface expression level of one or more IMRs in the one or more immune cell populations. In some cases, “expression level” of an IMR and “surface expression level” of an IMR are used synonymously herein. In general, “expression level,” when referring to an IMR, means surface expression level, unless otherwise indicated. The characterization may include characterizing the functional level of one or more pathways affected by the one or more IMRs/IMR pathways in one or more immune cell populations, indicative of the functional status of the IMR/IMR pathway or IMR pathways/IMR pathways; in some embodiments, this is done by determining levels of one or more intracellular activatable elements in single cells of the populations, such as the levels after modulation of the cells; in some embodiments it may be done by characterizing levels of one or more intracellular expressible elements in the cells. In some embodiment, the methods include modulation, e.g., activation, of one or more IMR/IMR pathways. In certain embodiments, the surface expression levels of one or more IMR ligands (IMRLs) or other components of cells associated with a pathological condition, such as cancer cells, e.g., tumor cells, may also be characterized. Other embodiments of the methods and compositions of the invention are as described below.

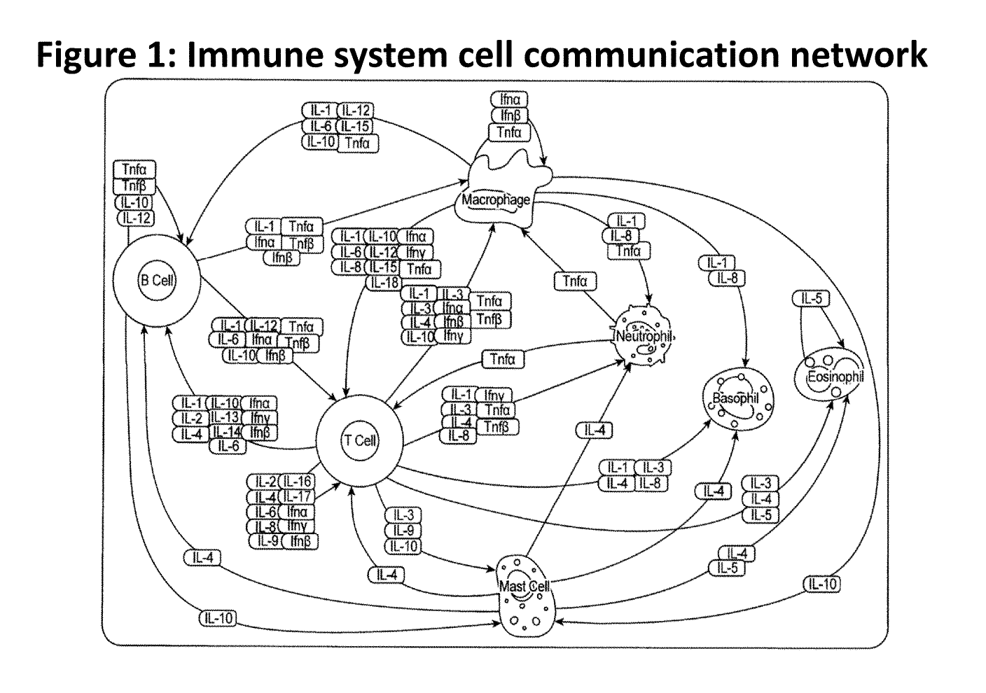

The activation levels of intracellular activatable elements can change in a matter of minutes, much faster than a change in expression levels can be detected, and can be measured at a time no greater than 2, 4, 6, 8, 10, 15, 20, 25, 30, 45, 60, 90, 120, 150, or 240 min., but also can be measured at later times, e.g., at least 2, 4, 6, 8, 10, 12, 16, 20, or 24 hours after exposure of the immune cells to activator, or at time that is greater than or equal to 2, 4, 6, 8, 10, 15, 20, 25, 30, 45, 60, 90, 120, 150, or 240 min. and less than or equal to 2, 4, 6, 8, 10, 12, 16, 20, 24, 30, 36, 42, 48, 54, 60, 66, or 72 hours after exposure of the immune cells to activator; changes in activation levels of the intracellular activatable elements at later time points often reflect intercellular communication between different immune cell populations, mediated at least in part by secretion of some of the intracellular expressed elements, such as cytokines, into the extracellular space and their influence on other immune cell populations. See FIG. 1. Intracellular activatable elements of use in the invention include any suitable activatable element; exemplary activatable elements are shown in Table 1 and described elsewhere herein, see, e.g., Activatable Elements, Signaling Pathways, and FIG. 20, and can include phosphoproteins and/or proteins activated by cleavage, also as described herein. For example, changes, e.g., increases or decreases, in p-ERK, pZAP70, PLCg, p-pKCtheta, p-p38 and/or p-AKT activation levels can correspond to immune cell, e.g., T-cell activation.

The intracellular expression levels of intracellular expressed elements change more slowly than activation levels of activation elements in response to activation of an immune cell population, and are measured hours or even days after the activation of the immune cell or cell population. Thus, the expression level of one or more intracellular expression elements may be determined at least 4, 8, 12, 16, 20, 24, 30, 36, 42, or 48 hours after activation of the immune cell or cell population. Typically, the expression level is compared with an expression level in an cell or population from the same immune cell population that was not activated; the difference is the expression level of the intracellular expressible element. Intracellular expressed elements of use in the invention include any suitable expressed element; exemplary expressed elements are shown in Table 1 and described elsewhere herein. One class of such intracellular expressed elements is cytokines, which include chemokines. For example, changes in intracellular expression level of one or more of IFNg, TNFa, and IL-2 may be used to determine the activation level of, e.g., T cells; see Examples, as well as further description herein. See also Table 1. Intracellular expression elements useful in the invention also include non-cytokine elements, such as cell cycle elements, e.g., Ki67 and Cyclin A2

Additionally, or alternatively, in certain embodiments a “surrogate activator” may be used to determine the functional status of one or more IMRs/IMR pathways in a cell or cell population, e.g., T cells, e.g., use of cytokine activator (surrogate activator) such as IL-6, IL-10, IL-15, IL-21, IL-2, IL-4, IL-12, IFNa, or IFNg, or any combination thereof, and measuring the activation level of an activatable element in the JAK/STAT pathway, such as p-STATs (e.g., 1, 3, 4, 5, or 6 or any combination thereof), with or without modulation of the IMR or IMRs of interest, in single cells of the population, e.g., T cell population. See Table 1, and Example 14. In certain embodiments, basal levels of the activatable element or element may alone be a surrogate for functional status of an IMR or IMRs, e.g., by comparison with a value derived from analysis of samples with known functional status of the IMR or IMRs. See Example 14.

Thus, determining the functional status of an IMR/IMR pathway can in certain embodiments entail the use of an activator of the immune cell population or populations of interest, an activator of the IMR/IMR pathway of interest, and the determination of either or both of a change in activation level of an intracellular activatable element, e.g., phosphoprotein, or change in the expression level of an intracellular expressed element, in response to contact of the cells of the cell population with the activator, and in the presence and absence of activation of the IMR/IMR pathway. In certain embodiments, a surrogate activator of the immune cells of the immune cell population is used. In certain embodiments, basal activation levels of one or more activatable elements are used as an indicator of IMR/IMR pathway status, alone or in combination with other elements as described herein.

IMRs are typically expressed in response to activation of an immune cell, and in quiescent cells one or more IMRs may be at very low levels, but in activated cells, one or more IMRs will be expressed on the surface of the cell in order to allow modulation of the now-active immune response. In general, immune cells from a sample from a patient suffering from a condition, e.g. cancer, already are activated, and IMRs are already expressed on their surface; indeed, this is one mechanism by which the cancer suppresses the immune response. In certain embodiments, immune cells from sample from an individual, e.g., a patient, such as a cancer patient, are used “as is”, without further significant modulation of the expression of their IMRs. In other embodiments, one or more modulations of the cells may be necessary in order to induce measurable or useful surface expression levels of one or more IMRs in cells, or otherwise prepare the cells for meaningful measurements. For example, cells from healthy individuals are usually quiescent, and expression of one or more IMRs can be induced, for example if the cells are to be used for screening agents that may affect IMRs/IMR pathways. Any suitable method to induce surface expression of one or more IMRs may be used; a preferred method is to activate the immune cells, which is the normal route by which expression of IMRs is induced, for a certain period of time, e.g., at least 24, at least 48 hours or any other suitable interval, to allow expression of the IMRs, then use the cells, e.g., to study functional status of the IMRs/IMR pathways in the cells, often after resting the cells for a period so that the initial activation of the cell can subside but IMR expression levels remain high enough to use. See Example 16 and FIG. 18.

Surface expression levels of one or more IMRs on cells of one or more immune cell populations may also be determined in certain embodiments of the invention. In certain embodiments, surface expression levels of at least 2, at least 3, at least 4, or at least 5, 6, 7, 8, 9, or 10 different IMRs may be determined in single cells of one or more immune populations, for example, determined on the same cell, and such levels for all the cells in a cell population or subpopulation used. The surface expression levels may be used alone, or in combination with other determinations, e.g., in combination with determination of the functional status of one or more IMRs, for example, at least 1, at least 2, at least 3, or at least 4, 5, 6, 7, 8, 9, or 10 different IMRs, e.g., determined in single cells of an immune cell population. The expression level of an IMR and the functional status of the same IMR, e.g., PD-1, may be measured on the same cell of an immune cell population. This determination may be used, e.g., to gate the cell into or out of a population. For example, when surface expression levels of one or more IMRs are measured in single cells of a population and the functional status of the IMR or IMRs is also measured in the same cells, it may be desirable to determine the functional status of the IMR or IMRs only in cells that are expressing the IMR on their surface at a level greater than, or greater than or equal to, a threshold level, and cells may first be gated into the population with such expression levels before the functional status of the IMR or IMR is determined.

Surface expression levels of one or more cell surface markers, useful in classifying cells as members of an immune cell population (e.g., T cell, B cell, etc.), or as members of a non-immune cell population (e.g., tumor cells) may also be determined in one or more embodiments of the invention. Tumor cell surface markers are well-known in the art, and any suitable marker or markers may be used. Similarly, immune cell surface markers are also well-known in the art and any suitable marker or markers may be used to place immune cells into one or more immune cell populations; exemplary cell surface markers and corresponding immune cell populations are shown in Table 1 and FIG. 17, and throughout the Description and Examples, but any suitable set of cell surface markers corresponding to any suitable immune cell populations may be used, as will be apparent to those of skill in the art. Cells can be gated into a particular population by well-known techniques based on their surface expression levels of particular cell surface markers.

Certain embodiments of the invention are directed to methods and compositions involving a patient suffering from a pathological condition. The pathological condition involves modulation of the patient's immune system b, for example, cells associated with the condition, for example, immunosuppression by tumor cells. The pathological condition may be cancer, e.g., any one of the known cancers or cancers described herein. For convenience, description herein often refers to cancer, but non-cancer pathological conditions such as autoimmune disease or HIV infection are included. In some embodiments, the pathological condition is classified by a designation that mainly or entirely is derived from the results of one or more measures of the functionality of one or more IMRs in one or more immune cell populations derived from a sample obtained from an individual, optionally including surface expression levels of an IMR or IMRs on immune cells or cell populations, and/or surface expression levels of an IMR or IMRL on cell of a non-immune cell population, and is not based entirely or mainly on traditional diagnostic criteria. That is the classification of the condition is condition-agnostic, e.g. cancer-agnostic and is, instead based at least in part on the above criteria.

Certain embodiments of the invention are directed to methods and compositions involving treating a patient suffering from a pathological condition, e.g., cancer, with a treatment. The treatment may be any treatment known or devised in the art. In certain embodiments, the treatment includes immunotherapy. Immunotherapies are as described elsewhere herein, and can include one or more of vaccines, therapies aimed at modulating one or more interactions between IMRs and IMRLs, or modulating the IMR pathway, e.g., checkpoint therapies such as anti-PD-1 therapies and/or anti-CTLA-4 therapies, adoptive immune cell therapy, cytokine therapy, and the like, as described elsewhere herein. In certain embodiments, the treatment is a combination treatment, that is, at least two different treatments, such as a combination immunotherapy treatment, such as one or more immunotherapies and one or more non-immunotherapy treatments, or two or different immunotherapy treatments, such as a vaccine and at least one other immunotherapy, such as modulation of one or more IMRs/IMR pathways. In certain embodiments, the treatment is a combination immunotherapy of two or more different immunotherapies in which one of the immunotherapies is modulation of an IMR/IMR pathway; or in which 2, 3, 4, or 5 or more of the immunotherapies is modulation of 2, 3, 4, or 5 or more different IMR/IMR pathways, such as the IMRs IMR pathways shown in FIG. 15. In certain embodiments, one of the pathways is the PD-1 pathway. In certain embodiments, one of the pathways is the CTLA-4 pathway. In certain embodiments, two of the pathways are the PD-1 pathway and the CTLA-4 pathway. In certain embodiments, the treatment comprises an immunotherapy comprising modulation of PD-1/PD-1 pathway, alone or in combination with modulation of one or more other IMR/IMR pathways, such as one or more of the IMRs/IMR pathways shown in FIG. 15 and the Description thereof, for example, CTLA-4/CTLA-4 pathway. In certain embodiments, the combination treatment is a combination of one or more immunotherapies and a non-immunotherapy treatment, such as one or more of a targeted treatment (a treatment specifically targeted at tumor cells such as MAb or conjugated MAb therapy), chemotherapy, radiation treatment, or surgical treatment. Such treatments are described in, e.g., Vanneman, M., and Dranoff, G., Nature Rev Cancer, 12:237-251 (2012).

In certain embodiments, the immunotherapy comprises an immunotherapy that comprises modulation of just one IMR/IMR pathway, such as one of the IMR/IMR pathways shown in FIG. 15 and the Description thereof. In certain embodiments, the treatment comprise modulation of the just the PD-1 pathway. In certain embodiments, the treatment comprises modulation of the just the CTLA-4 pathway. In certain embodiments, the treatment comprises modulation of the just the PD-1 pathway. In certain embodiments, the treatment comprises modulation of the just the LAG-3 pathway. In certain embodiments, the treatment comprises modulation of the just the TIM-3 pathway. In certain embodiments, the treatment comprises modulation of the just the VISTA pathway. In certain embodiments, the treatment comprises modulation of the just the GITR pathway. In certain embodiments, the treatment comprises modulation of the just the OX-40 pathway. In certain embodiments, the treatment comprises modulation of the just the CD27 pathway. In certain embodiments, the treatment comprises modulation of the just the 4-1BB pathway.

“Modulation of an IMR,” “modulation of an IMR pathway,” and equivalent terms, as used herein, refer to a modulation that specifically alters the IMR-IMRL interaction and its effects on immune cells on which the IMR is expressed, for example, contacting the IMR with an agonist, contacting the IMR with an antagonist (blocking the IMR), contacting the IMRL with a blocking agent, such as an antibody, and any other method of specifically altering the IMR-IMRL interaction and its effects on the immune cell.

In certain embodiments, the immunotherapy is treatment with an immunotherapy that comprises modulation of just two IMR/IMR pathways, such as two of the IMR/IMR pathways shown in FIG. 15 and the Description thereof. In certain embodiments, the treatment comprises modulation of the just the PD-1 pathway and one other IMR pathway. In certain embodiments, the treatment comprises modulation of the just the CTLA-4 pathway and one other IMR pathway. In certain embodiments, the treatment comprises modulation of the just the LAG-3 pathway and one other IMR pathway. In certain embodiments, the treatment comprises modulation of the just the TIM-3 pathway and one other IMR pathway. In certain embodiments, the treatment comprises modulation of the just the VISTA pathway and one other IMR pathway. In certain embodiments, the treatment comprises modulation of the just the GITR pathway and one other IMR pathway. In certain embodiments, the treatment comprises modulation of the just the OX-40 pathway and one other IMR pathway. In certain embodiments, the treatment comprises modulation of the just the CD27 pathway and one other IMR pathway. In certain embodiments, the treatment comprises modulation of the just the 4-1BB pathway and one other IMR pathway. In certain embodiments the treatment comprises modulating just the PD-1 pathway and just the CTLA-4 pathway. In certain embodiments the treatment comprises modulating just the PD-1 pathway and just the OX-40 pathway, for example, in an AML patient.

Certain embodiments of the invention are directed to methods and compositions involving one or more aspects of treating a patient with a treatment. An aspect of treating a patient with a treatment encompasses any element of the treatment that may affect treatment outcome, where treatment outcome includes the effect of the treatment on the pathological condition, and/or on the overall health and/or comfort of the patient. Examples of aspects of treatment include, but are not limited to, a decision to treat the patient or not treat the patient with a treatment or component of the treatment, choice of the treatment or a component of the treatment, a choice of the timing of the treatment or of a component of the treatment, a choice of a dosage of the treatment or a component of the treatment, or a combination thereof.

Certain embodiments of the invention are directed to methods and compositions involving a decision process, for example, a treatment decision process, generally engaged in by at least the patient and/or one or more of the patient's healthcare providers. A treatment decision process includes any process by which an outcome, e.g., an outcome regarding an aspect of treatment, is determined. Exemplary outcomes of a treatment decision process include a first likelihood of the patient responding to the treatment, a second likelihood of prolongation of the patient's life due to receiving the treatment, or a third likelihood of the patient experiencing an adverse treatment effect, or any combination of the first, second, and/or third likelihoods. In certain embodiments a treatment decision process includes consideration of a quantitative value, or at least 2, 3, 4, 5, 6, 7, 8, 9 or 10 quantitative values, or a value or values derived therefrom, or at least 2, 3, 4, 5, 6, 7, 8, 9 or 10 quantitative values derived therefrom. In certain embodiments a treatment decision process includes consideration of at least 3, 4, 5, 6, 7, 8, 9, or 10 quantitative values, or a value or values derived therefrom, or at least 3, 4, 5, 6, 7, 8, 9, or 10 values derived therefrom. Consideration of the quantitative value or values is generally engaged in by at least the patient and/or one or more of the patient's healthcare providers, and can comprise, for example, comparing the one or more quantitative values to a threshold value, e.g., comparing a quantitative value to a threshold value to decide whether or not the patient will respond to a particular treatment or component of a treatment, comparing the quantitative value to a continuous function, e.g., comparing it to a function that indicates probability of response of the patient to a treatment given the quantitative value or values, or a combination thereof. Comparison can also be done automatically, for example, so as to give a simple yes/no answer for the patient and/or healthcare provider(s) to consider, or to give a probability for the patient and/or healthcare provider(s) to consider, or any combination thereof. Any other comparison involving the quantitative value or values, or value or values derived therefrom, that can influence the outcome of a treatment decision process may also be used. Thus, as an example only, a decision to treat a patient may be made by a patient and his or her healthcare provider, where the decision process includes consideration of a quantitative value, or two quantitative values, or a quantitative value that is derived therefrom, or two quantitative values derived therefrom, where the quantitative value is determined at least in part by interrogating one or more immune cell populations from a sample from the patient, and considering the value using a classifier, e.g., a hierarchical classifier or a continuous function classifier such as a linear classifier, e.g., where the consideration indicates that the patient will respond to the treatment. The interrogating the one or more immune cell populations may comprise determining the functional status of one or more IMRs in the one or more immune cell populations; determining surface expression levels of the one or more IMRs.

To clarify decision processes involving quantitative values, an exemplary decision process is: A treatment decision process for whether or not to treat a patient with an immunotherapy may include comparing a first quantitative value to a first threshold value, such that if the first quantitative value is greater than or equal to the first threshold value, the patient is likely to respond to the immunotherapy (e.g., is a responder). Either the patient and/or their healthcare provider make the comparison, or the comparison may be made automatically, e.g., in the form of a simple yes/no. The first quantitative value corresponds to the functional status of an IMR pathway in cells of one or more immune cell populations from a sample from the patient. The first quantitative value is derived from a plurality of initial quantitative values, each of which represents, for a single cell in the one or more immune cell populations, an activation level of an intracellular activatable element in the single cell, for example, a signal magnitude from labels on antibodies that binds to the intracellular activatable elements in the particular cell. The derivation procedure may be complex, and involve one or more intermediate quantitative values between the plurality of initial values and the final first quantitative value, see, e.g., Table 2. Alternatively, a second quantitative value may also be obtained, for example, a quantitative value that corresponds to surface expression levels of the IMRL that activates the first IMR, on tumor cells from a sample from the patient. The second quantitative value may also be compared to a threshold, or the first and second quantitative values may be combined, e.g., with weighting of each value to reflect its relative importance in predicting response to the immunotherapy, to obtain a third value, and it is the third value that is used in the treatment decision process. In certain embodiments, a single quantitative value is used in the treatment decision process, and or a single quantitative value is used for each aspect of the treatment that the treatment decision process addresses.

Obtaining the threshold values and/or continuous functions to which a quantitative value is compared may be done, in the case of treatment, diagnosis, prognosis, or monitoring decision processes, through retrospective and/or prospective studies where, e.g., a training phase establishes putative threshold value(s) and/or continuous function(s), as well as any manipulations of quantitative values necessary to obtain a meaningful quantitative value for comparison, and a validation phase validates them. Such methods are well-known in the art.

A decision process, e.g., a prognostic, diagnostic, monitoring decision process, such as a treatment decision process, may also comprise consideration of a characteristic of the patient, such a genetic characteristic, age, gender, race, health status, previous treatment history, or any combination thereof. For example, certain immunotherapies are only given to patients with a certain genetic characteristic, such as the presence or absence of a gene mutation; e.g., cetuximab is only used in patients with wild-type (unmutated) KRAS genes. Thus an initial consideration in a treatment decision process may involve consideration of whether or not the patient has the relevant genetic mutation. Likewise, if the patient has received certain chemotherapies or other therapies, or a certain number or combination of such therapies, they may be more or less likely to respond to a certain immunotherapy. Any suitable characteristic, as known in the art or as discovered, related to a particular condition, e.g., pathological condition, from which an individual may suffer or potentially suffer, may be used in the methods and compositions of the invention.

A decision process, e.g., a prognostic, diagnostic, monitoring decision process, such as a treatment decision process, may comprise consideration of the number of cells in one or more immune cell populations, or a ratio of cell numbers in one immune cell population to the number of cells in another immune cell population. For example, a low Treg/Tcyto ratio in TILS is related to better overall prognosis.

Certain embodiments of the invention are directed to a prognosis decision process. A prognosis decision process includes any process by which an outcome, e.g., an outcome affecting a decision regarding a prognosis, is made. Exemplary outcomes of a prognosis decision process include a likelihood of a healthy individual developing a pathological condition, for example, within a certain period of time; a likelihood of a patient suffering from a pathological condition experiencing a worsening of the condition, e.g. within a certain period of time; and the like. The prognosis decision process is analogous to the treatment decision process, above, and any differences and/or modifications will be readily apparent to one of ordinary skill in the art; for example, the prognosis decision process can be partially or completely automated, can be performed by one or more of the individual's healthcare providers, etc.

Certain embodiments of the invention involve a drug screening decision process. A drug screening decision process includes any process by which one or more candidate therapeutic agents are determined to move or not move to a next level of screening, and can be engaged in by a person or persons, performed automatically, or any combination thereof.

Certain embodiments of the invention are directed to methods and compositions involving one or more quantitative values. Any or all of activation level of an intracellular activatable element, intracellular expression level of an intracellular expressed element, surface expression level of a cell surface marker, or other markers or characteristics described herein, and/or a change thereof, is expressed as a quantitative value in certain embodiments herein. Such quantitative values can be used to derive further quantitative values. Examples of quantitative values of use in the invention are given in Table 2, however a quantitative value derived from one or more such values may be used. In certain embodiments, a quantitative value or a derivative thereof (generally another quantitative value) is compared to a threshold value. The result of the comparison varies depending on the embodiment. In certain embodiments, the quantitative value is compared to a continuous function, such as a linear function, for example, to determine probability of response of a patient to treatment. As an example of the use of quantitative values, cells can be gated based on the results of comparison of one or more quantitative values or their derivative, e.g., values reflecting surface expression levels of cell surface markers, or surface expression levels of IMRs, may be used to gate cells. As another example, the selection of the treatment may be based on the outcome of a decision process that includes consideration of one or more quantitative values, that is indicative, e.g. of the probability that the patient will respond to a treatment, such as an immunotherapy, or one or more such treatments.

In certain embodiments of the invention, one or more of activation level of an intracellular activatable element, intracellular expression level of an intracellular expressed element, surface expression level of a cell surface marker, or other markers or characteristics described herein, is determined in single cells, for example single cells derived from a sample from a patient, such as a blood or a blood-derived sample, e.g., a peripheral blood mononuclear cell (PBMC), or from a bone marrow or bone marrow-derived sample, such as a bone marrow mononuclear sample (BMMC), or from a solid sample, such as a solid tumor sample, where, for example, the cells may include tumor-infiltrating lymphocytes (TILS) and/or tumor cells, and/or other tumor associated cells. A solid tumor sample may be a primary tumor sample or a metastatic tumor sample, and obtained, e.g., as a biopsy or during a surgical procedure, such as surgical resection of a tumor. Sample and methods of sampling are described in more detail elsewhere herein. Any suitable method may be used, as described herein, such as cytometry, for example, flow cytometry or mass cytometry. In certain embodiments, one or more distinguishably detectable binding elements is used in order to provide a detectable signal corresponding to a characteristic to be measured, e.g., the activation level of an intracellular activatable element in a cell. A “distinguishably detectable binding element” is a binding element, as that term is used herein, for example, an antibody or antibody fragment, that both binds to a component of interest, e.g., an activatable element in a particular activation state, and whose bound form can be detected, e.g., through a label, such as a fluorescent label for flow cytometry or a mass label, also referred to as a mass tag, in mass cytometry, that produces a signal that can be detected, e.g., by a cytometer. Its signal can be distinguished from that of any other detectable binding element used in the particular process in which it is used. The signal that is detected is a quantitative value and it may be manipulated to produce other quantitative values, see, e.g., Table 2. The values may be used to gate cells, as known in the art and as described herein. Gating may include an automatic component. Gating may include a manual component. In certain embodiments, gating includes both a manual and an automatic component; see, e.g., U.S. Patent Application No. 20130173618.

It will be appreciated that by using methods such as the above, treatment and treatment decisions, and or prognoses, or drug screening, may be partially or completely condition- (e.g. cancer-) agnostic; that is, a treatment is selected, or a prognosis formulated, or a candidate agent selected, not solely or mainly based on traditional patient phenotypes, e.g. traditional cancer phenotypes such as colon cancer, prostate cancer, ovarian cancer, renal cancer, cancer stages, cancer histology, etc., but solely or mainly based on a patient phenotype based on the state of immunomodulation, e.g., immunosuppression, in the patient, such as a phenotype based on the one or more of the cell population phenotypes described herein.

### Exemplary Embodiments of the Invention

In a first set of embodiments, the invention provides methods and compositions related to determining a functional status of an IMR in cells of a cell population, e.g., an immune cell population. The IMR may be any suitable IMR, for example of FIG. 15 and the description thereof, for which knowledge of a functional status is desired in a particular immune cell population, such as any of the immune cell populations described herein, e.g., of Table 1 or FIG. 17. The cells may be gated into the immune cell population by standard gating methods, e.g., by determining the surface expression levels of one or more cell surface markers of the cells and gating them accordingly. Exemplary cell surface markers are shown in Table 1 and FIG. 17, but any suitable cell surface markers may be used. In certain embodiments, the functional status of an IMR or IMRs may be determined in a plurality of cell populations, for example a plurality of immune cell populations.

The method can comprise contacting cells of the immune cell population or populations with an activator that activates the cells, in the presence and absence of activation of the IMR, e.g., in the presence and absence of an IMRL or an IMR agonist, and determining a change in the activation level of one or more intracellular activatable elements, as described herein, and/or the change in expression levels of one or more intracellular expressed elements, as described herein, for example, in single cells of the population. Any suitable IMRL or IMR agonist may be used to activate the IMR, such as one or more of those shown in FIG. 15 and the description thereof or Table 1, so long as it activates the particular IMR whose functional status is to be determined. Suitable intracellular activatable elements, e.g., phosphoproteins, include those shown in Table 1 and FIG. 20, for example, p-ERK and/or p-AKT.

The particular activator used to activate the cells, e.g. immune cells can depend on the immune cell population; for example, TCR activators will be specific to T cell populations, BCR activators will be specific to B cell populations; other activators, such as TLR agonists and ligands, may have a broader effect, activating cells of more than one immune cell population, or may be specific, in some cases. Any suitable activator may be used, such as one of those shown in Table 1, or a combination of activators. In certain embodiments, a surrogate activator, e.g., one or more cytokines, may be used, and the appropriate activatable element measured, such as one or more p-STATs (e.g., 1, 3, 4, 5, or 6), assessed.

The surface expression level of the IMR or IMRs may also be determined for the single cells, for example, in the same cell a surface expression level and a functional status of the same IMR may be determined. Surface expression levels of a different IMR, or a plurality of different IMRs, including others whose functional status is not determined, may be determined in the single cells in addition to, or alternatively to, the expression level of the IMR whose functional status is determined in the cell. The surface expression of the IMR may be used to gate the cells, so that the functional status of the IMR is determined only in cells expressing the IMR at a level greater than, or equal to or greater than, a threshold value.

The determination of functional status of the IMR or IMRs may also be determined when contacting the cells of the cell population with an agent, for example, an agent being screened as a potential therapeutic agent, or a therapeutic agent that can be used to treat a patient from whom the cells were derived, and the effect of the agent on the functional status of the IMR or IMRs determined.

Any suitable method of evaluating single cells may be used, for example, cytometry, such as flow cytometry or mass cytometry.

The functional status of a plurality of IMRs may also be determined. In certain embodiments, the IMRs are evaluated in the same cell, for example, if it is wished to determine if a certain combination therapy or combination agent that affects the plurality of IMR pathways may be effective, for example, in treating a patient from whom the cells have been derived. In certain embodiments, the functional status of a plurality of IMRs is determined, but at least some or all of the IMRs are evaluated on different cells, so as to determine the functional status of each IMR separately. This eliminates the additive or synergistic effects that may be seen when a plurality of IMRs are evaluated on the same cell, which can be desirable in some applications.

Certain sets of embodiments relate to determining a phenotype, such as for a cell population, e.g., an immune cell population or a non-immune cell population. For each of these embodiments, a second phenotype may also be determined, where the second phenotype is based, at least in part, on the first phenotype, for example, determining a phenotype for an individual, where the phenotype is based, at least in part, on a phenotype for an immune cell population in a sample or samples from the individual. Additionally or alternatively, a phenotype may be determined based, at least in part, on two or more different phenotypes determined in different sets of embodiments as presented herein, or non-immune cell populations, as presented herein, for example, determining a phenotype for an individual, where the phenotype is based, at least in part, on two or more different phenotypes determined in different sets of embodiments as presented herein for immune cell populations in a sample or samples from the individual, and/or a phenotype for non-immune cell populations, such as tumor cells, in a sample or samples from the individual, as presented herein. For example, an individual may present with a cancer, for example AML, and be phenotyped as PD-1 positive, OX-40 negative, meaning that one or more immune cell populations and/or tumor cell populations in the individual showed high functioning and/or expression for PD-1 but low or no functioning for OX-40. An aspect of treating the patient with an immunotherapy could be based, at least in part, on the phenotype; for example the PD-1+ OX-40− phenotype patient could be a candidate for treatment with a PD-1 pathway modulator but not for treatment with an OX-40 pathway modulator. A patient with a PD-1+OX-40+ phenotype could be a candidate for a combination treatment with both a PD-1 modulator and an OX-40 modulator. These examples are merely illustrative and embodiments of the invention embrace different type of phenotyping for use in various situations, so long as the phenotyping involves one or more of the sets of embodiments provided herein.

In a second set of embodiments, the invention provides methods and compositions related to determining a phenotype of a population of cells of an immune cell population, e.g., an immune cell population derived from a sample from a patient suffering from a pathological condition, such as cancer, comprising determining in single cells of the cell population a functional status of one or more IMRs expressed or potentially expressed on the surface of the immune population cells and determining the phenotype based on the functional status of the IMR or IMRs. The functional status of the IMR may be determined by any suitable method, for example, by any of the methods used in the first set of embodiments, optionally including methods in which surface expression levels of one or more IMRs, such as the one or more IMRs whose functional status is being determined, are also determined (for example, where the surface expression level and the functional level of an IMR are determined in the same cell), as well as, optionally, surface expression levels of one or more other IMRs, is determined; the expression levels thus determined can be used, e.g., in gating the cells, as described above, or as separate pieces of information about the cells of the immune cell population, or both. The sample can be any sample as described herein, for example, a PBMC sample, a BMMC sample, or a solid tumor sample; the immune cell population phenotype may be determined in TILS, such as TILS from a solid tumor sample or TILS in a blood or blood-derived sample. The immune cell population may be any immune cell population described herein, for example, an immune cell population of Table 1 or FIG. 17. In certain embodiments, the method comprises determining the phenotype based on functional statuses of 2 different IMRs, where the functional status of each IMR may be determined in separate cells, or the functional status of the 2 different IMRs determined in the same cell, optionally with determination of surface expression levels of one or both of the 2 different IMRs on the cells, e.g., in the same cells in which functional status is determined. It will be appreciated that in embodiments in which functional status of 2 or more IMRs are determined in the same cell, the results can be a single functional status that reflects the influence of both the IMRs, when activated, on the activation of the immune cells, whereas when functional status is determined for each IMR in separate cells, the results can be multiple functional statuses, each reflecting a different IMR. Combinations of the two different approaches may be used.

In certain embodiments, the method comprises determining the phenotype based on functional status of at least 3, 4, 5, 6, 7, 8, 9, or 10 different IMRs, where the functional status of each IMR may be determined in separate cells, or the functional status of the IMRs determined on the same cell, in any combination. In certain embodiments, the functional status of each IMR is determined in separate cells so that the functional status of one IMR is determined in any given cell. The IMR or IMRs may be any suitable IMR, such as IMRs shown in FIG. 15 and its accompanying description. In certain embodiments the invention includes treating a patient based on the phenotype thus determined for one or more immune cell populations derived from a sample obtained from the patient, where the treatment may be any treatment as described herein, such as an immunotherapy, e.g. a combination immunotherapy, for example an immunotherapy that is a combination of at least two immunotherapies. Exemplary treatments are given herein, and include a combination immunotherapy that includes modulation of the PD-1 pathway, or modulation of the CTLA-4 pathway, or both, or a monotherapy that involves modulation of any of the IMR pathways shown in FIG. 15 and its description, such as PD-1, or CTLA-4. In certain embodiments the invention includes treating a patient based on the phenotype thus determined, optionally also based on a phenotype as determined by, e.g., any of the methods used in the third set of embodiments, and optionally based on a phenotype as determined by, e.g., any of the methods used in the fourth set of embodiments, or a phenotype derived from the phenotypes (e.g., a phenotype of the patient), where the treatment may be any treatment as described herein, such as an immunotherapy, e.g. a combination immunotherapy, for example an immunotherapy that is a combination of at least two immunotherapies. Exemplary treatments are given herein, and include a combination immunotherapy that includes modulation of the PD-1 pathway, or modulation of the CTLA-4 pathway, or both, or a monotherapy that involves modulation of any of the IMR pathways shown in FIG. 15 and its description, such as PD-1, or CTLA-4.

In a third set of embodiments, the invention provides methods and compositions related to determining a phenotype of a population of cells of an non-immune cell population in a sample from, e.g., a patient suffering from a pathological condition, such as cancer, comprising determining in single cells of the cell population, e.g., tumor cells, surface expression levels of at least at least one, for example, at least two, such as at least three, different IMRLs and determining the phenotype based on the levels of the at least one, two, or three different IMRLs. In certain embodiments, the phenotype may be determined based on the surface expression levels of at least 4 different IMRLs on single cells of the population. In certain embodiments, the phenotype may be determined based on the surface expression levels of at least 5, 6, 7, 8, 9, or 10 different IMRLs on single cells of the population. The surface expression levels for the different IMRLS may be determined on the same cell for the cells of the non-immune cell population, as long as they may be distinguishably determined on the same cell; alternatively or additionally, the surface expression level of each may each be determined on different cells, or any combination of determination of any number on the same cell or different cells. The sample can be any sample as described herein, for example, a PBMC sample, a BMMC sample, or a solid tumor sample, and the non-immune cell population phenotype may be determined in tumor cells. The IMRLs may be any suitable IMRL, such as IMRLs shown in FIG. 15 and its accompanying description. In certain embodiments the invention includes treating a patient based on the phenotype thus determined, optionally also based on a phenotype as determined by, e.g., any of the methods used in the second set of embodiments, and optionally based on a phenotype as determined by, e.g., any of the methods used in the fourth set of embodiments, or a phenotype derived from the phenotypes (e.g., a phenotype of the patient), where the treatment may be any treatment as described herein, such as an immunotherapy, e.g. a combination immunotherapy, for example an immunotherapy that is a combination of at least two immunotherapies. Exemplary treatments are given herein, and include a combination immunotherapy that includes modulation of the PD-1 pathway, or modulation of the CTLA-4 pathway, or both, or a monotherapy that involves modulation of any of the IMR pathways shown in FIG. 15 and its description, such as PD-1, or CTLA-4.

In a fourth set of embodiments, the invention provides methods and compositions related to determining the phenotype of a population of cells of an immune cell population in a sample, for example, a sample from a patient suffering from a pathological condition, comprising determining in single cells of the immune cell population surface expression levels of at least one, for example two, such as at least three different IMRs, and determining the phenotype based on the surface expression levels of the at least one, two, or three different IMRs. The pathological condition can be cancer. The sample can be any sample as described herein, for example, a PBMC sample, a BMMC sample, or a solid tumor sample, and the immune cell population phenotype may be determined in cells that include TILS. The immune cell population may be any immune cell population described herein, for example, an immune cell population of Table 1 or FIG. 17. In certain embodiments, the method comprises determining the phenotype based on surface expression levels of at least 4 different IMRs on single cells of the population. In certain embodiments, the method comprises determining the phenotype based on surface expression levels of at least 5, 6, 7, 8, 9, or 10 different IMRs on single cells of the population. The IMRs may be any suitable IMR, such as IMRs shown in FIG. 15 and its accompanying description. The surface expression levels for the different IMRS may be determined on the same cell for the cells of the non-immune cell population, as long as they may be distinguishably determined on the same cell; alternatively or additionally, the surface expression level of each may each be determined on different cells, or any combination of determination of any number on the same cell or different cells. In certain embodiments the invention includes treating a patient based on the phenotype thus determined, optionally also based on a phenotype as determined by, e.g., any of the methods used in the second set of embodiments, and optionally based on a phenotype as determined by, e.g., any of the methods used in the third set of embodiments, or a phenotype derived from the phenotypes (e.g., a phenotype of the patient), where the treatment may be any treatment as described herein, such as an immunotherapy, e.g. a combination immunotherapy, for example an immunotherapy that is a combination of at least two immunotherapies. Exemplary treatments are given herein, and include a combination immunotherapy that includes modulation of the PD-1 pathway, or modulation of the CTLA-4 pathway, or both, or a monotherapy that involves modulation of any of the IMR pathways shown in FIG. 15 and its description, such as PD-1, or CTLA-4.

In a fifth set of embodiments, the invention provides methods and compositions related to treating a patient suffering from a pathological condition including treating the patient with a treatment for the condition, wherein an aspect of treating the patient with the treatment is based on an outcome of a treatment decision process comprising consideration of a first quantitative value, or a value or values derived from the first quantitative value, wherein the first quantitative value is obtained from results of an assay comprising determining functional status of one or more IMRs in single cells of a immune cell population or a subpopulation thereof in a sample from the patient. The pathological condition may be any suitable pathological condition as described herein, e.g., cancer. Determining the functional status of the one or more IMRs may be accomplished by any suitable method, such one or more of the methods used in the first set of embodiments. The treatment may be any suitable treatment, such as any suitable treatment described herein, such as treatments described in the second, third, or fourth sets of embodiments, or any other suitable treatment, such as a combination treatment that includes an immunotherapy and also includes one or more of a targeted therapy, radiation therapy, surgical therapy, or chemotherapy, or a combination treatment that includes two different immunotherapies, such as vaccine and modulation of one or more IMRs/IMR pathways, and the like.

The methods may also comprise determining surface expression levels of the IMR or IMRs in the single cells, for example, by any of the methods used in the first set of embodiments, for example, using cytometry, such as flow cytometry or mass cytometry. The expression levels may be used to gate cells into populations in which functional status is determined, for example to gate cells into a subpopulation of the immune cell population, and wherein single cells of the subpopulation are gated into the subpopulation on the basis of the surface expression levels of the IMR or IMRs of the single cell. The expression levels of the IMR or IMRs may, in addition or alternatively, be used to obtain a second quantitative value or values, which may also be considered in the treatment decision process.

The methods may also comprise determining surface expression levels of an IMRL or IMRLs, for example in single cells of a non-immune cell population, for example, a tumor cell population that can be derived from a sample from the patient, for example, by any of the methods used in the third set of embodiments. The expression levels of the IMRL or IMRLs maybe used to obtain a third quantitative value or values, which may also be considered in the treatment decision process, e.g., in a process where the first quantitative value is considered, or the first and second quantitative values are considered. Treatment decision processes, outcomes of treatment decision process, and aspects of treating the patient, may be any suitable process or processes, outcome or outcomes, and/or aspect or aspects, for example as described herein. For example, an aspect of treating the patient may comprise a decision to treat the patient or not treat the patient with the treatment, a choice of the treatment or a component of the treatment, a choice of the timing of the treatment or of a component of the treatment, a choice of a dosage of the treatment or a component of the treatment, or a combination thereof. As another example, an outcome of the treatment decision process may comprise a first likelihood of the patient responding to the treatment, a second likelihood of prolongation of the patient's life due to receiving the treatment, or a third likelihood of the patient experiencing an adverse treatment effect, or any combination of the first, second, and/or third likelihoods.

In a sixth set of embodiments, the invention provides methods and compositions related to treating a patient suffering from a pathological condition, e.g., cancer, comprising treating the patient with a treatment for the condition; wherein an aspect of treating the patient with the treatment is based on an outcome of a treatment decision process, wherein the treatment decision process comprises consideration of at least two of a first, second, and third quantitative value, or a value or values derived from the at least two quantitative values; and
wherein the first, second, and/or third quantitative values are obtained from results of a first, second, and/or third assay, respectively, wherein
   (a) the first assay comprises determining surface expression levels of a first immunomodulatory receptor (IMR) of a first cell population cell population (CP in a first sample from the patient;
   (b) the second assay comprises determining functional status of a second IMR in single cells of a second CP or a subpopulation thereof in a second sample from the patient; and
   (c) the third assay comprises determining surface expression levels of an IMR ligand (IMRL) for a third IMR in a third cell population in a third sample from the patient.

When surface expression levels of the first IMR are used, the expression levels may be determined in single cells, by any suitable method, for example, by any of the methods used the first set of embodiments, using any suitable detection technique, such as cytometry, e.g., flow cytometry or mass cytometry.

When functional status of the second IMR as determined in single cells is used, the functional status may be determined in single cells by any suitable method, for example, by any of the methods used the first set of embodiments, using any suitable detection technique, such as cytometry, e.g., flow cytometry or mass cytometry.

When surface expression levels of an IMRL for a third IMR is used, the levels may be determined in single cells, by any suitable method, for example, by any of the methods used the third set of embodiments, using any suitable detection technique, such as cytometry, e.g., flow cytometry or mass cytometry.

Quantitative values and their use in a treatment decision process, outcomes of treatment decision processes, aspects of treatment, and treatments, can be as described in the fifth set of embodiments. For example, an aspect of treating the patient comprises a decision to treat the patient or not treat the patient with the treatment, a choice of the treatment or a component of the treatment, a choice of the timing of the treatment or of a component of the treatment, a choice of a dosage of the treatment or a component of the treatment, or a combination thereof. As another example, an outcome of the treatment decision process comprises a first likelihood of the patient responding to the treatment, a second likelihood of prolongation of the patient's life due to receiving the treatment, or a third likelihood of the patient experiencing an adverse treatment effect, or any combination of the first, second, and/or third likelihoods, etc.

In certain embodiments in this sixth set of embodiments, assays comprise the first assay and the second assay, wherein the assays are performed on single cells, the first and second samples are the same sample, the first and second IMRs are the same IMR, and the first and second cell populations are the same population, and wherein the second quantitative value represents a functional status of the IMR for the subpopulation of the population, wherein the process of obtaining the second quantitative value comprises gating the results for functional status of the IMR in the single cells of the cell population on the basis of the results of the determination of the expression level of the IMR in the same single cells of the population. In certain embodiments, the gating comprises establishing a threshold for expression level of the IMR in a single cell and single cells in the cell population having an expression level of the IMR above the threshold are included in the subpopulation and single cells in the cell population having an expression level equal to or below, or below, the threshold are excluded from the subpopulation.

In certain embodiments of this sixth set of embodiments, the first and second cell populations are immune cell populations, for example, the first and second immune cell populations can be the same immune cell population or the first and second immune cell populations can be different immune cell populations. The third cell population can be a non-immune cell population, such as a tumor cell population. The first and second cell populations can comprise a first and second cell immune cell population of TABLE 1 or FIG. 17. The cell populations can be identified by any suitable method, e.g., by surface expression levels of at least one, two, three of the cell surface markers of Table 1 or FIG. 17.

In certain embodiments of this sixth set of embodiments the first sample and the second sample, and optionally the third sample, are the same sample. In all embodiments, the first, second and third samples can be any suitable samples, as described herein, for example, a blood or blood-derived sample, such as a PBMC sample, or bone marrow or bone marrow-derived sample, such as a BMMC, or a solid sample or solid-sample-derived samples, such as a tumor sample, for example a primary tumor sample or a metastatic tumor sample. Thus, in certain embodiments, the first and second samples comprise tumor-infiltrating lymphocytes (TILS) derived from a solid tumor sample and the third sample comprises tumor cells derived from the same solid tumor sample. However, in this and other embodiments of the invention, TILS may also be found in a blood or blood-derived sample, such as a PBMC sample, a bone or bone marrow-derived sample, such as a BMMC. Similarly, tumor cells may be found in a blood or blood-derived sample, such as circulating tumor cells (CTCs)

In certain embodiments of this sixth set of embodiments, in c) a plurality of IMRLs is determined, e.g., in single cells. The plurality of IMRLs can comprise a plurality of IMRLS of FIG. 15 and the description thereof.

In certain embodiments of this sixth set of embodiments, in a) and b) a plurality of IMRs is assayed. The plurality of IMRs can comprise a plurality of IMRs of FIG. 15 and the description thereof.

In certain embodiments of this sixth set of embodiments, the condition is cancer, the therapy is a combination therapy comprising immunotherapy, in a) and b) a plurality of IMRs is assayed, and the aspect of the treatment comprises choice of the combination therapy.

In certain embodiments of this sixth set of embodiments, as described in other embodiments, the treatment decision process further comprises consideration of a characteristic of the patient, such as a genetic characteristic, age, gender, race, health status, previous treatment history, or any combination thereof. Characteristics of the patient are as further described herein.

In certain embodiments of this sixth set of embodiments, the IMRL corresponds to the IMR for a) or b).

In certain embodiments of this sixth set of embodiments, the assay of part (b) comprises determining the functional status of the IMR in the presence and absence of an immunotherapeutic agent, or determining the functional status of the IMR in the presence and absence of a plurality of immunotherapeutic agents, such as immunotherapeutic agent or agents that are candidates for use in the treatment, or agents that represent a class of immunotherapeutic agents that are candidates for use in the treatment.

In a seventh set of embodiments, the invention provides methods and compositions related to a pharmaceutical package comprising one or more immunotherapeutic agents and
(i) instructions and/or an imprint indicating that the one or more immunotherapeutic agents is to be used for treatment of a patient who suffers from a pathological condition, e.g., cancer, wherein either
   (a) cells associated with the patient's pathological condition, e.g., tumor cells, are characterized by surface expression of an IMRL at a level greater than, or greater than or equal to a threshold level of expression or surface expression of a plurality of different IMRLs at levels greater than, or greater than or equal to, a plurality of threshold expression levels; or
   (b) an immune cell population from a sample from the patient is characterized by surface expression level of a first IMR that is greater than, or greater than or equal to a threshold expression level; or
   (c) an immune cell population from a sample from the patient is characterized by a change in the expression level and/or activation level of an intracellular element that is less than, or less than or equal to a threshold change, wherein the change in the expression level or activation level of the intracellular element in a cell of an immune cell type is in response to contact with an activator of that immune cell type and is indicative of the activation level of the cell, and wherein the change in the level may be measured in the presence and/or absence of an activator and/or inhibitor of an IMR that can be expressed on the cell of the immune cell type; or
   (c) a non-cell liquid from a sample from the patient contains an immune effector molecule at a level greater than, greater than or equal to, less than, or less than or equal to a threshold level; or
   (d) any combination of (a), (b), and/or (c); and/or
(ii) instructions and/or an imprint indicating that the patient is to be stratified by one or more the methods described herein that produces a result that can be used to determine if condition (i)(a), (b), (c), and/or (d) is satisfied; and/or
(iii) one or more necessary materials to carry out the one or more of methods of part (ii).

In certain embodiments of the seventh set of embodiments, the pharmaceutical package may further comprise one or more components for use in gathering, treating, and/or transporting one or more samples from the patient for use in the one or more methods of part (ii). In certain embodiments in which the pathological condition is cancer, the cancer can be characterized by tumor cell surface expression of an IMRL that modulates an inhibitory IMR of FIG. 15, for example, PD-1 and the description thereof, wherein the tumor cell surface expression level of the IMRL is greater than, or greater than or equal to, a threshold level. In certain embodiments in which the pathological condition is cancer, the cancer can be characterized by tumor cell surface expression of plurality of IMRLs, each of which modulates a different inhibitory IMR of FIG. 15, such as an IMRL that activates PD-1 and an IMRL that activates CTLA-4, and the description thereof, wherein the surface expression level of each of the IMRLs is greater than, or greater than or equal to, a threshold level for surface expression for that IMRL.

Intracellular activatable elements and their assay are as described in the methods and compositions used in the first set of embodiments. In certain embodiments, the intracellular activatable element comprises p-ERK, p-AKT, p-ZAP70, PLCg, p-PKCθ, p-p38, or pNFkBp65, such as p-ERK or p-AKT. In certain embodiments, the intracellular activatable element comprises p-STAT1, p-STAT3, p-STAT4, p-STAT5, or p-STAT6, or a combination thereof.

In an eighth set of embodiments, the invention provides methods and compositions related to screening a first agent, for example an agent for potential use in treatment of a pathological condition, such as cancer, at a first screening level comprising
(i) contacting a first immune cell population expressing a first IMR on their surfaces with the first agent and activating the cells of the first population by contacting them with an activator;
(ii) activating the cells of a second immune cell population expressing the first IMR on their surfaces that have not been contacted with the first agent by contacting them with the activator.
(iii) determining
   (a) expression levels of an intracellular expression element in single cells of the first population or a subpopulation thereof and expression levels of the intracellular element in single cells of the second population or a subpopulation thereof, wherein the intracellular expression element is an element whose expression levels changes upon activation of the cells of the first and second immune cell populations, and/or
   (b) activation levels of an intracellular activatable element in single cells of the first population or a subpopulation thereof and activation levels of the intracellular activatable element in single cells of the second population or a subpopulation thereof, wherein the intracellular activatable element is an activatable element whose activation level changes upon activation of the a cell of the first and second immune cell populations;
(iv) making a determination to send or not send the agent to a second screening level based on the results of (iii).

In certain embodiments of the eighth set of embodiments, the determination of step (iv) can comprise an evaluation of a result of a comparison of the expression levels of the intracellular element and/or the activation levels of the intracellular activatable element in the single cells of the first population, or a first quantitative value derived therefrom, with the expression levels of the intracellular element and/or the activation levels of the intracellular activatable element in the single cells of the second population, or a second quantitative value derived therefrom. the result can be a third quantitative value. The determination of step (iv) can comprise comparing the third quantitative value with a threshold value to determine if the third value is greater than, greater than or equal to, less than, or less than or equal to the threshold value. The agent can be sent to the second screening level if the third quantitative value is greater than, or greater than or equal to, the threshold value. Alternatively, the agent can be sent to the second screening level if the third quantitative value is less than, or less than or equal to, the threshold value.

In certain embodiments of the eighth set of embodiments, the first and second cell populations can the same immune cell population, or they can be different immune cell populations. The identity of the first and second immune cell populations can be determined by determining the levels of at least one cell surface marker in single cells of the first and second immune cell populations.

In certain embodiments of the eighth set of embodiments, the method can further comprise determining the expression levels of the intracellular element and/or the activation levels of the intracellular activatable element in single cells of a third immune cell population type that have not been activated and that have not been contacted with the agent. The first, second, and third immune cell populations can be the same immune cell population, or one or more of them can be different from the others.

In certain embodiments of the eighth set of embodiments, the method can further comprise determining surface expression levels of the first IMR in single cells of the first and second immune cell populations, for example the expression levels of the intracellular element and/or the activation levels of the intracellular activatable element can be determined in subpopulations of the first and second immune cell populations, and a cell is gated into the subpopulation of the first or second population on the basis of its surface expression level of the first IMR. A cell can be gated by comparing its surface expression level of the IMR to a threshold expression level value for the first IMR, wherein the cell is gated into the subpopulation if its surface expression level of the first IMR is greater than the threshold value, or greater than or equal to the threshold value. See, e.g., compositions and methods described for use in the first set of embodiments.

In certain embodiments of the eighth set of embodiments, the method can further comprising screening a second agent in combination with the first agent wherein the second agent is different from the first agent and wherein the cells of the first immune cell population further express a second IMR on their surfaces and step (i) further comprises contacting the first immune cell population with the second agent; and the cells of the second immune cell population further express the second IMR on their surfaces and in step (ii) the cells of the second population have not been contacted with the second agent. The method can further comprise determining surface expression levels of the second IMR in single cells of the first and second immune cell populations. Expression levels of the intracellular expression element and/or the activation levels of the intracellular activatable element can be determined in subpopulations of the first and second populations, and a cell can be gated into the subpopulation of the first and second population on the basis of its surface expression level of the first IMR and its surface expression level of the second IMR. A cell can be gated by comparing its surface expression level of the first IMR to a threshold expression level value for the first IMR and its surface expression level of the second IMR to a threshold expression level value for the second IMR, wherein the cell is gated into the subpopulation if its surface expression level of the first IMR is greater than the threshold value for the surface expression level of the first IMR and its surface expression level of the second IMR is greater than the threshold value for the surface expression level of the second IMR, or greater than or equal to the threshold values for the surface expression of the first and second IMRs.

In certain embodiments of the eighth set of embodiments, the cells of the first and second immune cell populations expressing the first IMR, and, optionally, second IMR, have been induced to express the first IMR, and, optionally, second IMR, by activation of the cells of the first and second immune cell populations at a time previous to steps (i) and (ii). Methods of induction of IMRs are as described in the methods used in the first set of embodiments of the invention. The cells can be, e.g., derived from a sample from a healthy individual, a plurality of samples from the healthy individual, or a plurality of samples from a plurality of healthy individuals.

In certain embodiments of the eighth set of embodiments, the cells can be from cell lines.

In certain embodiments of the eighth set of embodiments, the cells can derived from a sample from an individual suffering from a pathological condition, e.g., cancer, or a plurality of samples from the individual, or a plurality of samples from a plurality of individuals suffering from the pathological condition, e.g., cancer.

In certain sets of embodiments, the invention provides kits. In these sets of embodiments, a kit may comprise:
(i) at least 1, 2, 3, or 4, for example in certain embodiments at least 1, such as in certain embodiments at least 2, distinguishably detectable binding elements for determination of activation levels of at least 1, 2, 3, or 4, for example in certain embodiments at least 1, such as in certain embodiments at least 2, intracellular activatable elements whose levels indicate the functional status of one or more IMRs; and/or
(ii) at least 1, 2, 3, or 4, for example in certain embodiments at least 1, such as in certain embodiments at least 2, further such as in certain embodiments at least 3 distinguishably detectable binding elements for determining intracellular expression levels of one or more intracellular expressed elements that indicate the functional status of one or more IMRs; and/or
(iii) at least 1, 2, 3, 4, 5, 6, 7, 8, 9, or 10, for example in certain embodiments at least 1, in certain embodiments at least 2, in certain embodiments at least 3, in certain embodiments at least 4, in certain embodiments at least 5, distinguishably detectable binding elements for determining surface expression levels of one or more different IMRs; and/or
(iv) at least 1, 2, 3, 4, 5, 6, 7, 8, 9, or 10, for example in certain embodiments at least 1, in certain embodiments at least 2, in certain embodiments at least 3, in certain embodiments at least 4, in certain embodiments at least 5, distinguishably detectable binding elements for determining surface expression levels of one or more different IMRLs; and/or
(v) activators for at least 1, 2, 3, 4, 5, 6, 7, 8, 9, or 10, for example in certain embodiments at least 1, in certain embodiments at least 2, in certain embodiments at least 3, in certain embodiments at least 4, in certain embodiments at least 5, different IMRs; and/or
(vi) activators for) at least 1, 2, 3, or 4, for example in certain embodiments at least 1, such as in certain embodiments at least 2, further such as at least 3 different immune cell populations; and/or
(vii) at least 1, 2, 3, or 4, for example in certain embodiments at least 1, in certain embodiments at least 2, 1, 2, 3, 4, 5, 6, 7, 8, 9, or 10, for example in certain embodiments at least 1, in certain embodiments at least 2, in certain embodiments at least 3, in certain embodiments at least 4, in certain embodiments at least 5, distinguishably detectable binding elements for determining surface expression levels of one or more different cell surface markers that indicate the classification of an immune cell into an immune cell population and/or the classification of a non-immune cell into a non-immune cell population.

Distinguishably detectable binding elements can be any suitable distinguishably detectable binding elements, such as those described herein. For example, the distinguishably detectable binding elements can be antibodies, as that term is defined herein, which can be labeled, for example, directly labeled, with distinguishably detectable labels suitable for detection in a flow cytometer, for example, fluorophores. As another example, the distinguishably detectable binding elements can be antibodies, as that term is defined herein, which can be labeled, for example, directly labeled, with distinguishably detectable labels suitable for detection in a mass cytometer, for example, mass labels or mass tags. Exemplary mass labels are those used for detection in the CyToF instruments, available from Fludigm.

Any of the kits described herein can contain the elements of the kit as described above and can further include, if the kit does not already include it, one or more of the following:
(a) instructions for use of the kit, where the instructions may be written or available electronically, e.g., on a website;
(b) suitable packaging, e.g., packaging suitable for transport from a kit manufacturer or distributor to an end user, where a single kit may be packaged in one or more than one packages, so long as all the packages are for use for a single purpose, which may be indicated, e.g., on a website, or in a set of instructions, or the like;
(c) reagents for use in conducting the procedure or procedures for which the elements of the kit are intended, such as buffers, permeabilizers, fixatives, and the like, as described elsewhere herein;
(d) components for use in for use in conducting the procedure or procedures for which the elements of the kit are intended, such as microtiter plates, e.g. 96-well plates, which can be unloaded or preloaded with one or more elements of the kit, buffers, etc.;
(e) material for interpreting the results of use of the kit, such as scientific papers, where the materials may be written or available electronically, e.g., on a website;
(f) software for use in one or more procedures associated with the kit, where the software may be provided in any suitable form, such as a computer readable medium or downloadable or a combination thereof.

For example, a kit may be a kit intended for use in a companion diagnostic process for a therapeutic agent, such as an immunotherapeutic agent for use in an immunotherapy comprising modulation of one or more IMR pathways, e.g., an IMR modulator, or IMRL modulator, where the kit includes a distinguishably detectable antibody configured for use in binding to and detecting an activatable element, where the magnitude of change in the activation level of the activatable element in single cells of an immune population derived from a sample from a patient in whom the immunotherapeutic agent may be used corresponds to the functional level of an IMR pathway, and instructions for use, where the antibody is suitably packaged for transport from a manufacturer or a distributor to an end user and the instructions for use are suitably configured to be used by the end user, such as written instructions including with or separate from the kit, instructions on a website, and the like, to accomplish the purpose of the kit, which may be to provide results suitable for determining if a given patient will or will not respond to the therapeutic agent, or the probability that the patient will respond, or that the patient will or will not suffer an adverse event if given the immunotherapeutic agent, or the probability thereof, and the like, as described more fully elsewhere herein.

The latter is merely exemplary of a relatively simple kit; kits can comprise a plurality of components in any combination as described above, so long as they are supplied from a manufacturer or distributor to an end user for a particular use, e.g., in immunotherapy. The particular use may be to produce one or more results that is used in a decision process, such as a decision process described herein, e.g., a treatment decision process, a prognosis, diagnosis, or monitoring process, a screening process, etc.

In a ninth set of embodiments, the invention provides a kit comprising
(i) a distinguishably detectable binding element configured for use in binding to and distinguishably detecting a first intracellular element, wherein a change in the expression level and/or activation level of the first intracellular element in a cell of an immune cell type in response to exposure of the cell to an activator of the immune cell type is indicative of activation of the cell; and
(ii) a distinguishably detectable binding element configured for use in binding to and distinguishably detecting a cell surface IMR on the cell or a cell surface IMRL on a cell of a population of cells of a non-immune cell type. The kit can further comprise the activator.

In certain embodiments of the ninth set of embodiments, the kit includes a plurality of distinguishably detectable binding elements configured for use in binding to and distinguishably detecting a plurality of different cell surface IMRs or a plurality of different cell surface IMRLs. The plurality of different surface IMRs and/or the plurality of different cell surface IMRLs can be a plurality of different surface IMRs and/or a plurality of different cell surface IMRLs of FIG. 15 and the description thereof. For example, the plurality of IMRs can comprise PD-1 and CTLA-4 and the plurality of IMRLs can comprise at least two of B7-1, B7-2, PDL-1, and PDL-2.

In certain embodiments of the ninth set of embodiments, the kit further comprises instructions for use of the kit, for example in an assay for predicting the response of a patient to immunotherapy, such as wherein the immunotherapy is an immunotherapy that directly or indirectly affects activation of the population of cells of the immune cell type.

In certain embodiments of the ninth set of embodiments, the kit further comprises a plurality of distinguishably detectable binding elements, each configured for use in binding to and distinguishably detecting a different cell surface marker, wherein the level of at least two of the plurality of different cell surface markers can be used to type the cell as a cell of an immune cell population. The plurality of cell surface markers can comprise any suitable plurality, as known in the art, for example, a plurality of cell surface markers listed in TABLE 1 or FIG. 17.

In certain embodiments of the ninth set of embodiments, the cell surface IMR or the cell surface IMRL comprises an IMR or an IMRL or of FIG. 15 and the description thereof. The IMR can be PD-1 and the IMRL can be PDL-1 or PDL-2.

In certain embodiments of the ninth set of embodiments, the intracellular element is an intracellular activatable element, such as an activatable element of TABLE 1 or FIG. 20.

In a tenth set of embodiments, the invention provides a kit comprising at least one, for example in certain embodiments at least two, such as in certain embodiments at least three, distinguishably detectable binding elements, wherein the at least one, two, or three distinguishably detectable binding elements are configured for use in binding to and distinguishably detecting at least one, two, or three different cell surface IMRs on single cells of an immune cell population and/or at least one, two, or three cell surface IMRLs on single cells of a non-immune cell population, e.g., a tumor cell population

In certain embodiments of the tenth set of embodiments, the kit comprises at least four distinguishably detectable binding elements, wherein the at least four distinguishably detectable binding elements are configured for use in binding to and distinguishably detecting at least one, two, three, or four different cell surface IMRs on single cells of an immune cell population and/or at least one, two, three, or four cell surface IMRLs on single cells of a non-immune cell population.

In certain embodiments of the tenth set of embodiments, the kit comprises at least five distinguishably detectable binding elements, wherein the at least five distinguishably detectable binding elements are configured for use in binding to and distinguishably detecting at least one, two, three, four, or five different cell surface IMRs on single cells of an immune cell population and/or at least one, two, three, four or five cell surface IMRLs on single cells of a non-immune cell population.

In certain embodiments of the tenth set of embodiments, the kit comprises at least six distinguishably detectable binding elements, wherein the at least six distinguishably detectable binding elements are configured for use in binding to and distinguishably detecting at least one, two, three, four, five, or six different cell surface IMRs on single cells of an immune cell population and/or at least one, two, three, four, five, or six surface IMRLs on single cells of a non-immune cell population.

In certain embodiments of the tenth set of embodiments, the kit comprises at least seven distinguishably detectable binding elements, wherein the at least seven distinguishably detectable binding elements are configured for use in binding to and distinguishably detecting at least one, two, three, four, five, six, or seven different cell surface IMRs on single cells of an immune cell population and/or at least one, two, three, four, five, six, or seven surface IMRLs on single cells of a non-immune cell population.

In certain embodiments of the tenth set of embodiments, the kit comprises at least eight distinguishably detectable binding elements, wherein the at least eight distinguishably detectable binding elements are configured for use in binding to and distinguishably detecting at least one, two, three, four, five, six, seven, or eight different cell surface IMRs on single cells of an immune cell population and/or at least one, two, three, four, five, six, seven, or eight surface IMRLs on single cells of a non-immune cell population.

In certain embodiments of the tenth set of embodiments, the kit comprises both one or more distinguishably detectable binding elements configured for use in binding to and distinguishably detecting at least one, two, three, four, five, six, seven, or eight different cell surface IMRs on single cells of an immune cell population and one or more distinguishably detectable binding elements configured for use in binding to and distinguishably detecting at least one, two, three, four, five, six, seven, or eight cell surface IMRLs on single cells of a non-immune cell population, e.g., a tumor cell population.

In certain embodiments of the tenth set of embodiments, the surface IMRs and/or cell surface IMRLs can be surface IMRs and/or cell surface IMRLs of FIG. 15 and the description thereof. For example, the IMRs can comprise PD-1 and CTLA-4 and the IMRLs can comprise at least two of B7-1, B7-2, PDL-1, and PDL-2.

In an eleventh set of embodiments, the invention provides methods and compositions related to a system.

In certain embodiments of the eleventh set of embodiments, the invention provides a system for treating a patient suffering from a pathological condition, e.g., cancer, with a treatment, wherein the system comprises
(i) the patient
(ii) a healthcare provider for the patient;
(iii) a first sample from the patient and, optionally, a second sample from the patient;
(iv) a system for determining a quantitative value, or a value or values derived from the quantitative value, wherein the quantitative value is obtained from results of an assay comprising determining functional status of an IMR, for example, at least 1, 2, 3, 4, 5, 6, 7, 8, 9, or 10 IMRs, in single cells of an immune cell population in the first sample from the patient, and/or a quantitative value, or a value or values derived from the quantitative value, wherein the quantitative value is obtained from results of an assay comprising determining surface expression levels of one or more IMRLs in single cells of a non-immune cell population in the second sample, where the first and second samples may be the same or different, e.g., at least 1, 2, 3, 4, 5, 6, 7, 8, 9, 10 IMRLS;
(v) a transport system to transport the sample or samples to the site of the assay;
(vi) a communication system to communicate the quantitative value, values, or a value or values derived from the quantitative value or values, to the patient and/or the healthcare provider, and/or to a system wherein the value or values may be further analyzed and/or modified;
wherein an aspect of treating the patient with the treatment is based on an outcome of a treatment decision process comprising consideration by the patient and/or healthcare provider of the quantitative value, or the value or values derived from the quantitative value, or consideration by the patient and/or healthcare provider of the results of the further analysis and/or modification.

In a twelfth set of embodiments, the invention provides a system

In certain embodiments of the twelfth set of embodiments, the invention provides a system for screening potential agents for immunotherapy comprising
(i) a person or persons, or entity, that desires to know the outcome of the screening;
(ii) a system for determining a result or results of the screening, such as a screening described in any one or more of the methods and compositions of the eighth set of embodiments;
(iii) a communication system to communicate the result or results to the person or persons or entity and/or to a system wherein the result or results may be further analyzed and/or modified to produce a further result or results and the result or results communicated to the person or persons or entity.

In a thirteenth set of embodiments, the invention provides methods and compositions related to determining a vaccine therapy for a patient suffering from a pathological condition, e.g., cancer, comprising
(i) determining in an immune cell population, e.g., DC cells or a population derived therefrom, from a sample obtained from the patient, a functional status of an IMR or IMRs, e.g., at least 1, 2, 3, 4, 5, 6, 7, 8, 9, 10 IMRs, in single cells of the sample; and
(ii) determining the vaccine therapy based, at least in part, on the results of (i).

### Samples and Sampling

The invention may involve analysis of one or more samples from an individual. An individual or a patient is any multi-cellular organism; in some embodiments, the individual or patient is an animal, e.g., a mammal. In some embodiments, the individual or patient is a human.

The sample may be any suitable type that allows for the methods of the invention. In general, a solid sample, such as a tumor biopsy, or a liquid sample, such as a blood or blood-derived, e.g. PBMC sample, or both, is used. The advantage of a blood or blood-derived sample is that it is easy to obtain and often stored so that retrospective studies can be performed, and it can reflect, e.g., the tumor microenvironment. In methods in which single cells are analyzed, the sample may be any suitable type that allows for the analysis of single cells. Samples may be obtained once or multiple times from an individual. Multiple samples may be obtained from different locations in the individual (e.g., blood samples, bone marrow samples, lymph node samples, biopsies, and/or resection material), at different times from the individual (e.g., a series of samples taken to monitor response to treatment or to monitor for return of a pathological condition), or any combination thereof.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals of approximately 1-7 days, for example, every 1, 2, 3, 4, 5, 6, or 7 days, or at irregular intervals of 1, 2, 3, 4, 5, 6, or 7 days, or at intervals of 1-4 weeks, for example, every 1, 2, or 3 weeks, or at irregular intervals of 1, 2, 3, or 4 weeks, or at intervals of approximately 1-12 months, at intervals of approximately 1, 2, 3, 4, 5, or more than 5 years, or any combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cancer may be sampled (e.g., by blood draw) relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, peripheral blood mononuclear cells (PBMCs), bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passageways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion). In some embodiments the sample or samples is blood.

In certain embodiments, a solid tissue sample is used. Solid tissue samples may also be used, either alone or in conjunction with fluid samples. One example of a solid tissue sample is a tumor sample. Tumor samples contain tumor cells and, generally, immune cells such as tumor infiltrating lymphocytes, and it is of interest to determine characteristics of one or both of these cell types. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

Samples may include circulating tumor cells (CTC). Methods for isolating CTC are known in the art. See for example: Toner M et al. Nature 450, 1235-1239 (20 Dec. 2007); Lustenberger P et al. Int J Cancer. 1997 Oct. 21; 74(5):540-4; Reviews in Clinical Laboratory Sciences, Volume 42, Issue 2 Mar. 2005, pages 155-196; and Biotechno, pp. 109-113, 2008 International Conference on Biocomputation, Bioinformatics, and Biomedical Technologies, 2008.

In certain embodiments, the sample is a blood or PMBC sample. Blood and PBMC samples are particularly suited to analysis of hematopoietic cancers such as leukemia and lymphoma; however, PBMC samples can also be used with solid tumors if the status of the circulating immune cells can be correlated, e.g. with a treatment outcome. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used. For certain types and locations of cancer, a lavage or perfusion may be used, e.g., for lung, bladder, stomach, colon and other cancer sites may provide sufficient immune and/or tumor cells for an analysis.

In one embodiment, a sample may be obtained from an apparently healthy individual during a routine checkup and analyzed so as to provide an assessment of the individual's general health status. In another embodiment, a sample may be taken to screen for commonly occurring diseases. Such screening may encompass testing for a single disease, a family of related diseases or a general screening for multiple, unrelated diseases. Screening can be performed once, weekly, bi-weekly, monthly, bi-monthly, every several months, annually, or in several year intervals and may replace or complement existing screening modalities.

In another embodiment, an individual with a known increased probability of disease occurrence may be monitored regularly to detect for the appearance of a particular disease or class of diseases. An increased probability of disease occurrence can be based on familial association, age, previous genetic testing results, or occupational, environmental or therapeutic exposure to disease causing agents. Breast and ovarian cancer related to inherited mutations in the genes BRCA1 and BRCA2 are examples of diseases with a familial association wherein susceptible individuals can be identified through genetic testing. Another example is the presence of inherited mutations in the adenomatous polyposis coli gene predisposing individuals to colorectal cancer. Examples of environmental or therapeutic exposure include individuals occupationally exposed to benzene that have increased risk for the development of various forms of leukemia, and individuals therapeutically exposed to alkylating agents for the treatment of earlier malignancies. Individuals with increased risk for specific diseases can be monitored regularly for the first signs of an appearance of an abnormal discrete cell population. Monitoring can be performed once, weekly, bi-weekly, monthly, bi-monthly, every several months, annually, or in several year intervals, or any combination thereof. Monitoring may replace or complement existing screening modalities. Through routine monitoring, early detection of the presence of disease causative or associated cells may result in increased treatment options including treatments with lower toxicity and increased chance of disease control or cure.

In a further embodiment, testing can be performed to confirm or refute the presence of a suspected genetic or physiologic abnormality associated with increased risk of disease. Such testing methodologies can replace other confirmatory techniques like cytogenetic analysis or fluorescent in situ histochemistry (FISH). See U.S. Ser. No. 12/688,851. In still another embodiment, testing can be performed to confirm or refute a diagnosis of a pre-pathological or pathological condition.

In instances where an individual has a known pre-pathologic or pathologic condition, one or more samples may be obtained and analyzed to predict the response of the individual to available treatment options, or to determine the optimal treatment. In one embodiment, an individual treated with the intent to reduce in number or ablate cells that are causative or associated with a pre-pathological or pathological condition can be monitored to assess the decrease in such cells over time. A reduction in causative or associated cells may or may not be associated with the disappearance or lessening of disease symptoms. If the anticipated decrease in cell number does not occur, further treatment with the same or a different treatment regiment may be warranted. In addition, or alternatively, as described elsewhere herein, the immunological profile of the individual may be monitored, for example, during and after immunotherapy, to determine the effectiveness of the treatment in terms of immune system function, as well as to monitor for any changes that indicate that the treatment effect is declining.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regiment can be considered.

Individuals may also be monitored for the appearance or increase in cell number of another discrete cell population(s) that are associated with a good prognosis. If a beneficial, discrete cell population is observed, measures can be taken to further increase their numbers, such as the administration of growth factors. Alternatively, individuals may be monitored for the appearance or increase in cell number of another discrete cell population(s) associated with a poor prognosis. In such a situation, renewed therapy can be considered including continuing, modifying the present therapy or initiating another type of therapy.

Certain fluid samples can be analyzed in their native state with or without the addition of a diluent or buffer. Alternatively, fluid samples may be further processed to obtain enriched or purified cell populations prior to analysis. Numerous enrichment and purification methodologies for bodily fluids are known in the art. A common method to separate cells from plasma in whole blood is through centrifugation using heparinized tubes. By incorporating a density gradient, further separation of the lymphocytes from the red blood cells can be achieved. A variety of density gradient media are known in the art including sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells can be filtered out of diluted, whole blood following the lysis of red blood cells by using filters with pore sizes between 5 to 10 μm, as disclosed in U.S. patent application Ser. No. 09/790,673. Alternatively, whole blood can be separated into its constituent cells based on size, shape, deformability or surface receptors or surface antigens by the use of a microfluidic device as disclosed in U.S. patent application Ser. No. 10/529,453.

Select cell populations can also be enriched for or isolated from whole blood through positive or negative selection based on the binding of antibodies or other entities that recognize cell surface or cytoplasmic constituents. For example, U.S. Pat. No. 6,190,870 to Schmitz et al. discloses the enrichment of tumor cells from peripheral blood by magnetic sorting of tumor cells that are magnetically labeled with antibodies directed to tissue specific antigens.

Solid tissue samples may require the disruption of the extracellular matrix or tissue stroma and the release of single cells for analysis. Various techniques are known in the art including enzymatic and mechanical degradation employed separately or in combination. An example of enzymatic dissociation using collagenase and protease can be found in Wolters G H J et al. An analysis of the role of collagenase and protease in the enzymatic dissociation of the rat pancrease for islet isolation. Diabetologia 35:735-742, 1992. Examples of mechanical dissociation can be found in Singh, N P. Technical Note: A rapid method for the preparation of single-cell suspensions from solid tissues. Cytometry 31:229-232 (1998). Alternately, single cells may be removed from solid tissue through microdissection including laser capture microdissection as disclosed in Laser Capture Microdissection, Emmert-Buck, M. R. et al. Science, 274(8):998-1001, 1996. See also U.S. Patent Publication Number 20130129681, for descriptions of method for release of single cells from solid tissue samples.

In some embodiments, single cells can be analyzed within a tissue sample, such as a tissue section or slice, without requiring the release of individual cells before determining step is performed.

The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 12/432,720 and 13/493,857 and U.S. Pat. No. 8,227,202. See also, the commercial products from companies such as BD and BCI.

See also U.S. Pat. Nos. 7,381,535 and 7,393,656. All of the above patents and applications are incorporated by reference as stated above.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 30%.

### Activatable Elements

An “activatable element” is an element, e.g, a protein, that can exist in two or more states. In general, activation can result in a change in the activatable element, e.g., protein, that results in a conformation that is detectably different from the non-activated form. An example is a phosphoprotein, which can exist in one or, in some cases, more than one phosphorylated forms, and a nonphosphorylated form. Another example is a protein that is activated by cleavage, where the cleaved protein can be considered an activated form. For convenience, one form can be designated the “activated” form, and another an “unactivated” form; though there can be several forms, similar principles apply.

Typically, a cell possesses a plurality of a particular activatable element, some of which are in the activated form and some of which are in the unactivated form. One form or both forms can be distinguishably detectable, for example, the activated form may be distinguishably detectable, for example through binding of a binding element that is specific to the activated form. When the cell is exposed to the distinguishably detectable binding elements, only those activatable elements in the activated form are recognized by the binding element, representing some fraction of the total number of activatable elements, and generate a measurable signal. The measurable signal corresponding to the summation of individual activated elements of a particular type that are activated in a single cell can be the “activation level” for that activatable element in that cell. In certain instances an activatable element may be referred to in its unactivated form, and in certain instances in its activated form; in general, the two are synonymous, and either may be considered the “activatable element.”

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

Cellular constituents that may include activatable elements include without limitation proteins, carbohydrates, lipids, nucleic acids and metabolites. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

One example of a covalent modification is the substitution of a phosphate group for a hydroxyl group in the side chain of an amino acid (phosphorylation). A wide variety of proteins are known that recognize specific protein substrates and catalyze the phosphorylation of serine, threonine, or tyrosine residues on their protein substrates. Such proteins are generally termed “kinases.” Substrate proteins that are capable of being phosphorylated are often referred to as phosphoproteins (after phosphorylation). Once phosphorylated, a substrate phosphoprotein may have its phosphorylated residue converted back to a hydroxyl one by the action of a protein phosphatase that specifically recognizes the substrate protein. Protein phosphatases catalyze the replacement of phosphate groups by hydroxyl groups on serine, threonine, or tyrosine residues. Through the action of kinases and phosphatases a protein may be reversibly phosphorylated on a multiplicity of residues and its activity may be regulated thereby. Thus, the presence or absence of one or more phosphate groups in an activatable protein is a preferred readout in the present invention.

Another example of a covalent modification of an activatable protein is the acetylation of histones. Through the activity of various acetylases and deacetlylases the DNA binding function of histone proteins is tightly regulated. Furthermore, histone acetylation and histone deactelyation have been linked with malignant progression. See Nature, 2004 May 27; 429(6990): 457-63.

Another form of activation involves cleavage of the activatable element. For example, one form of protein regulation involves proteolytic cleavage of a peptide bond. While random or misdirected proteolytic cleavage may be detrimental to the activity of a protein, many proteins are activated by the action of proteases that recognize and cleave specific peptide bonds. Many proteins derive from precursor proteins, or pro-proteins, which give rise to a mature isoform of the protein following proteolytic cleavage of specific peptide bonds. Many growth factors are synthesized and processed in this manner, with a mature isoform of the protein typically possessing a biological activity not exhibited by the precursor form. Many enzymes are also synthesized and processed in this manner, with a mature isoform of the protein typically being enzymatically active, and the precursor form of the protein being enzymatically inactive. This type of regulation is generally not reversible. Accordingly, to inhibit the activity of a proteolytically activated protein, mechanisms other than “reattachment” can be used. For example, many proteolytically activated proteins are relatively short-lived proteins, and their turnover effectively results in deactivation of the signal. Inhibitors may also be used. Among the enzymes that are proteolytically activated are serine and cysteine proteases, including cathepsins and caspases respectively.

Activation of an activatable element can involve prenylation of the element. By “prenylation”, and grammatical equivalents used herein, is meant the addition of any lipid group to the element. Common examples of prenylation include the addition of farnesyl groups, geranylgeranyl groups, myristoylation and palmitoylation. In general these groups are attached via thioether linkages to the activatable element, although other attachments may be used.

The activatable element can be a protein. Examples of proteins that can be activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in U.S. Publication Number 20060073474 entitled “Methods and compositions for detecting the activation state of multiple proteins in single cells” and U.S. Publication Number 20050112700 entitled “Methods and compositions for risk stratification” the content of which are incorporate here by reference. See also U.S. Ser. Nos. 12/432,720 and 12/229,476; and Shulz et al., Current Protocols in Immunology 2007, 7:8.17.1-20.

Exemplary proteins that may be activated include HER receptors, PDGF receptors, FLT3 receptor, Kit receptor, FGF receptors, Eph receptors, Trk receptors, IGF receptors, Insulin receptor, Met receptor, Ret, VEGF receptors, erythropoetin receptor, thromobopoetin receptor, CD114, CD116, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGF β receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK 3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon gamma, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-(tilde over the beta) catenin, FOXO, STAT1, STAT 3, STAT 4, STAT 5, STAT 6, p53, WT-1, HMGA, pS6, 4EPB-1, eIF4E-binding protein, RNA polymerase, initiation factors, and elongation factors.

An activatable element can be a nucleic acid. Activation and deactivation of nucleic acids can occur in numerous ways including, but not limited to, cleavage of an inactivating leader sequence as well as covalent or non-covalent modifications that induce structural or functional changes. For example, many catalytic RNAs, e.g. hammerhead ribozymes, can be designed to have an inactivating leader sequence that deactivates the catalytic activity of the ribozyme until cleavage occurs. An example of a covalent modification is methylation of DNA. Deactivation by methylation has been shown to be a factor in the silencing of certain genes, e.g. STAT regulating SOCS genes in lymphomas. See Leukemia. See February 2004; 18(2): 356-8. SOCS1 and SHP1 hypermethylation in mantle cell lymphoma and follicular lymphoma: implications for epigenetic activation of the Jak/STAT pathway. Chim C S, Wong K Y, Loong F, Srivastava G.

An activatable element can be a small molecule, carbohydrate, lipid or other naturally occurring or synthetic compound capable of having an activated isoform.

The activation level of an activatable element in a cellular pathway, or signaling pathway, can be determined.

### Signaling Pathways

In some embodiments, the methods of the invention are employed to determine the status of an activatable element in a signaling pathway. Signaling pathways for IMRs are the main pathways of interest in the invention; however, one or more pathways may be related to an IMR pathway or otherwise affected by it, for example, a pathway for activation of a cell, e.g., an immune cell. Signaling pathways and their members have been described. Exemplary signaling pathways include the following pathways and their members: the JAK-STAT pathway including JAKs, STATs 1, 2, 3 4, 5, and 6 the FLT3L signaling pathway, the MAP kinase pathway including Ras, Raf, MEK, ERK and Elk; the PI3K/Akt pathway including PI3-kinase, PDK1, Akt and Bad; the NF-κB pathway including IKKs, IkB and NF-κB, and the Wnt pathway including frizzled receptors, beta-catenin, APC and other co-factors and TCF.

One classification of signaling pathways, and exemplary elements of the pathways, including activatable element, is shown in FIG. 20.

Nuclear Factor-kappaB (NF-κB) Pathway:

Nuclear factor-kappaB (NF-kappaB) transcription factors and the signaling pathways that activate them are central coordinators of innate and adaptive immune responses. More recently, it has become clear that NF-kappaB signaling also has a critical role in cancer development and progression. NF-kappaB provides a mechanistic link between inflammation and cancer, and is a major factor controlling the ability of both pre-neoplastic and malignant cells to resist apoptosis-based tumor-surveillance mechanisms. In mammalian cells, there are five NF-κB family members, RelA (p65), RelB, c-Rel, p50/p105 (NF-κB1) and p52/p100 (NF-κB2) and different NF-κB complexes are formed from their homo and heterodimers. In most cell types, NF-κB complexes are retained in the cytoplasm by a family of inhibitory proteins known as inhibitors of NF-κB (IκBs). Activation of NF-κB typically involves the phosphorylation of IκB by the IκB kinase (IKK) complex, which results in IκB ubiquitination with subsequent degradation. Thus, either p-IkB or non-phosphorylated IkB can serves as an activatable element in this pathway. This releases NF-κB and allows it to translocate freely to the nucleus. The genes regulated by NF-κB include those controlling programmed cell death, cell adhesion, proliferation, the innate- and adaptive-immune responses, inflammation, the cellular-stress response and tissue remodeling. However, the expression of these genes is tightly coordinated with the activity of many other signaling and transcription-factor pathways. Therefore, the outcome of NF-κB activation depends on the nature and the cellular context of its induction. For example, it has become apparent that NF-κB activity can be regulated by both oncogenes and tumor suppressors, resulting in either stimulation or inhibition of apoptosis and proliferation.

Phosphatidylinositol 3-Kinase (PI3-K)/AKT Pathway:

PI3-Ks are activated by a wide range of cell surface receptors to generate the lipid second messengers phosphatidylinositol 3,4-biphosphate (PIP₂) and phosphatidylinositol 3,4,5-trisphosphate (PIP₃). Examples of receptor tyrosine kinases include but are not limited to FLT3 LIGAND, EGFR, IGF-1R, HER2/neu, VEGFR, and PDGFR. The lipid second messengers generated by PI3Ks regulate a diverse array of cellular functions. The specific binding of PI3,4P₂ and PI3,4,5P₃ to target proteins is mediated through the pleckstrin homology (PH) domain present in these target proteins. One key downstream effector of PI3-K is Akt, a serine/threonine kinase, which is activated when its PH domain interacts with PI3, 4P₂ and PI3,4,5P₃ resulting in recruitment of Akt to the plasma membrane. Once there, in order to be fully activated, Akt is phosphorylated at threonine 308 by 3-phosphoinositide-dependent protein kinase-1 (PDK-1) and at serine 473 by several PDK2 kinases. Akt then acts downstream of PI3K to regulate the phosphorylation of a number of substrates, including but not limited to forkhead box O transcription factors, Bad, GSK-3β, I-κB, mTOR, MDM-2, and S6 ribosomal subunit. These phosphorylation events in turn mediate cell survival, cell proliferation, membrane trafficking, glucose homeostasis, metabolism and cell motility. Deregulation of the PI3K pathway occurs by activating mutations in growth factor receptors, activating mutations in a PI3-K gene (e.g. PIK3CA), loss of function mutations in a lipid phosphatase (e.g. PTEN), up-regulation of Akt, or the impairment of the tuberous sclerosis complex (TSC1/2). All these events are linked to increased survival and proliferation.

Wnt Pathway:

The Wnt signaling pathway describes a complex network of proteins well known for their roles in embryogenesis, normal physiological processes in adult animals, such as tissue homeostasis, and cancer. Further, a role for the Wnt pathway has been shown in self-renewal of hematopoietic stem cells (Reya T et al., Nature. 2003 May 22; 423(6938):409-14). Cytoplasmic levels of β-catenin are normally kept low through the continuous proteosomal degradation of β-catenin controlled by a complex of glycogen synthase kinase 3β (GSK-3 β), axin, and adenomatous polyposis coli (APC). When Wnt proteins bind to a receptor complex composed of the Frizzled receptors (Fz) and low density lipoprotein receptor-related protein (LRP) at the cell surface, the GSK-3/axin/APC complex is inhibited. Key intermediates in this process include disheveled (Dsh) and axin binding the cytoplasmic tail of LRP. Upon Wnt signaling and inhibition of the β-catenin degradation pathway, β-catenin accumulates in the cytoplasm and nucleus. Nuclear β-catenin interacts with transcription factors such as lymphoid enhanced-binding factor 1 (LEF) and T cell-specific transcription factor (TCF) to affect transcription of target genes

Protein Kinase C (PKC) Signaling:

The PKC family of serine/threonine kinases mediates signaling pathways following activation of receptor tyrosine kinases, G-protein coupled receptors and cytoplasmic tyrosine kinases. Activation of PKC family members is associated with cell proliferation, differentiation, survival, immune function, invasion, migration and angiogenesis. Disruption of PKC signaling has been implicated in tumorigenesis and drug resistance. PKC isoforms have distinct and overlapping roles in cellular functions. PKC was originally identified as a phospholipid and calcium-dependent protein kinase. The mammalian PKC superfamily consists of 13 different isoforms that are divided into four subgroups on the basis of their structural differences and related cofactor requirements cPKC (classical PKC) isoforms (α, βI, βII and γ), which respond both to Ca2+ and DAG (diacylglycerol), nPKC (novel PKC) isoforms (δ, ε, θ and η), which are insensitive to Ca2+, but dependent on DAG, atypical PKCs (aPKCs, t/λ, ζ), which are responsive to neither co-factor, but may be activated by other lipids and through protein-protein interactions, and the related PKN (protein kinase N) family (e.g. PKN1, PKN2 and PKN3), members of which are subject to regulation by small GTPases. Consistent with their different biological functions, PKC isoforms differ in their structure, tissue distribution, subcellular localization, mode of activation and substrate specificity. Before maximal activation of its kinase, PKC requires a priming phosphorylation which is provided constitutively by phosphoinositide-dependent kinase 1 (PDK-1). The phospholipid DAG has a central role in the activation of PKC by causing an increase in the affinity of classical PKCs for cell membranes accompanied by PKC activation and the release of an inhibitory substrate (a pseudo-substrate) to which the inactive enzyme binds. Activated PKC then phosphorylates and activates a range of kinases. The downstream events following PKC activation are poorly understood, although the MEK-ERK (mitogen activated protein kinase kinase-extracellular signal-regulated kinase) pathway is thought to have an important role. There is also evidence to support the involvement of PKC in the PI3K-Akt pathway. PKC isoforms probably form part of the multi-protein complexes that facilitate cellular signal transduction. Many reports describe dysregulation of several family members. For example alterations in PKCε have been detected in thyroid cancer, and have been correlated with aggressive, metastatic breast cancer and PKCt was shown to be associated with poor outcome in ovarian cancer.

Mitogen Activated Protein (MAP) Kinase Pathways:

MAP kinases transduce signals that are involved in a multitude of cellular pathways and functions in response to a variety of ligands and cell stimuli. (Lawrence et al., Cell Research (2008) 18: 436-442). Signaling by MAPKs affects specific events such as the activity or localization of individual proteins, transcription of genes, and increased cell cycle entry, and promotes changes that orchestrate complex processes such as embryogenesis and differentiation. Aberrant or inappropriate functions of MAPKs have now been identified in diseases ranging from cancer to inflammatory disease to obesity and diabetes. MAPKs are activated by protein kinase cascades consisting of three or more protein kinases in series: MAPK kinase kinases (MAP3Ks) activate MAPK kinases (MAP2Ks) by dual phosphorylation on S/T residues; MAP2Ks then activate MAPKs by dual phosphorylation on Y and T residues MAPKs then phosphorylate target substrates on select S/T residues typically followed by a proline residue. In the ERK1/2 cascade the MAP3K is usually a member of the Raf family. Many diverse MAP3Ks reside upstream of the p38 and the c-Jun N-terminal kinase/stress-activated protein kinase (JNK/SAPK) MAPK groups, which have generally been associated with responses to cellular stress. Downstream of the activating stimuli, the kinase cascades may themselves be stimulated by combinations of small G proteins, MAP4Ks, scaffolds, or oligomerization of the MAP3K in a pathway. In the ERK1/2 pathway, Ras family members usually bind to Raf proteins leading to their activation as well as to the subsequent activation of other downstream members of the pathway.

a. Ras/RAF/MEK/ERK Pathway:

Classic activation of the RAS/Raf/MAPK cascade occurs following ligand binding to a receptor tyrosine kinase at the cell surface, but a vast array of other receptors have the ability to activate the cascade as well, such as integrins, serpentine receptors, heterotrimeric G-proteins, and cytokine receptors. Although conceptually linear, considerable cross talk occurs between the Ras/Raf/MAPK/Erk kinase (MEK)/Erk MAPK pathway and other MAPK pathways as well as many other signaling cascades. The pivotal role of the Ras/Raf/MEK/Erk MAPK pathway in multiple cellular functions underlies the importance of the cascade in oncogenesis and growth of transformed cells. As such, the MAPK pathway has been a focus of intense investigation for therapeutic targeting. Many receptor tyrosine kinases are capable of initiating MAPK signaling. They do so after activating phosphorylation events within their cytoplasmic domains provide docking sites for src-homology 2 (SH2) domain-containing signaling molecules. Of these, adaptor proteins such as Grb2 recruit guanine nucleotide exchange factors such as SOS-1 or CDC25 to the cell membrane. The guanine nucleotide exchange factor is now capable of interacting with Ras proteins at the cell membrane to promote a conformational change and the exchange of GDP for GTP bound to Ras. Multiple Ras isoforms have been described, including K-Ras, N-Ras, and H-Ras. Termination of Ras activation occurs upon hydrolysis of RasGTP to RasGDP. Ras proteins have intrinsically low GTPase activity. Thus, the GTPase activity is stimulated by GTPase-activating proteins such as NF-1 GTPase-activating protein/neurofibromin and p120 GTPase activating protein thereby preventing prolonged Ras stimulated signaling. Ras activation is the first step in activation of the MAPK cascade. Following Ras activation, Raf (A-Raf, B-Raf, or Raf-1) is recruited to the cell membrane through binding to Ras and activated in a complex process involving phosphorylation and multiple cofactors that is not completely understood. Raf proteins directly activate MEK1 and MEK2 via phosphorylation of multiple serine residues. MEK1 and MEK2 are themselves tyrosine and threonine/serine dual-specificity kinases that subsequently phosphorylate threonine and tyrosine residues in Erk1 and Erk2 resulting in activation. Although MEK1/2 have no known targets besides Erk proteins, Erk has multiple targets including Elk-1, c-Ets1, c-Ets2, p90RSK1, MNK1, MNK2, and TOB. The cellular functions of Erk are diverse and include regulation of cell proliferation, survival, mitosis, and migration. McCubrey, J. Roles of the Raf/MEK/ERK pathway in cell growth, malignant transformation and drug resistance. Biochimica et Biophysica Acta. 2007; 1773: 1263-1284, hereby fully incorporated by reference in its entirety for all purposes, Friday and Adjei, Clinical Cancer Research (2008) 14, p 342-346.

b. c-Jun N-Terminal Kinase (JNK)/Stress-Activated Protein Kinase (SAPK) Pathway:

The c-Jun N-terminal kinases (JNKs) were initially described as a family of serine/threonine protein kinases, activated by a range of stress stimuli and able to phosphorylate the N-terminal transactivation domain of the c-Jun transcription factor. This phosphorylation enhances c-Jun dependent transcriptional events in mammalian cells. Further research has revealed three JNK genes (JNK1, JNK2 and JNK3) and their splice-forms as well as the range of external stimuli that lead to JNK activation. JNK1 and JNK2 are ubiquitous, whereas JNK3 is relatively restricted to brain. The predominant MAP2Ks upstream of JNK are MEK4 (MKK4) and MEK7 (MKK7). MAP3Ks with the capacity to activate JNK/SAPKs include MEKKs (MEKK1, -2, -3 and -4), mixed lineage kinases (MLKs, including MLK1-3 and DLK), Tpl2, ASKs, TAOs and TAK1. Knockout studies in several organisms indicate that different MAP3Ks predominate in JNK/SAPK activation in response to different upstream stimuli. The wiring may be comparable to, but perhaps even more complex than, MAP3K selection and control of the ERK1/2 pathway. JNK/SAPKs are activated in response to inflammatory cytokines; environmental stresses, such as heat shock, ionizing radiation, oxidant stress and DNA damage; DNA and protein synthesis inhibition; and growth factors. JNKs phosphorylate transcription factors c-Jun, ATF-2, p53, Elk-1, and nuclear factor of activated T cells (NFAT), which in turn regulate the expression of specific sets of genes to mediate cell proliferation, differentiation or apoptosis. JNK proteins are involved in cytokine production, the inflammatory response, stress-induced and developmentally programmed apoptosis, actin reorganization, cell transformation and metabolism. Raman, M. Differential regulation and properties of MAPKs. Oncogene. 2007; 26: 3100-3112, hereby fully incorporated by reference in its entirety for all purposes.

c. p38 MAPK Pathway:

Several independent groups identified the p38 Map kinases, and four p38 family members have been described (α, β, γ, δ). Although the p38 isoforms share about 40% sequence identity with other MAPKs, they share only about 60% identity among themselves, suggesting highly diverse functions. p38 MAPKs respond to a wide range of extracellular cues particularly cellular stressors such as UV radiation, osmotic shock, hypoxia, pro-inflammatory cytokines and less often growth factors. Responding to osmotic shock might be viewed as one of the oldest functions of this pathway, because yeast p38 activates both short and long-term homeostatic mechanisms to osmotic stress. p38 is activated via dual phosphorylation on the TGY motif within its activation loop by its upstream protein kinases MEK3 and MEK6. MEK3/6 are activated by numerous MAP3Ks including MEKK1-4, TAOs, TAK and ASK. p38 MAPK is generally considered to be the most promising MAPK therapeutic target for rheumatoid arthritis as p38 MAPK isoforms have been implicated in the regulation of many of the processes, such as migration and accumulation of leucocytes, production of cytokines and pro-inflammatory mediators and angiogenesis, that promote disease pathogenesis. Further, the p38 MAPK pathway plays a role in cancer, heart and neurodegenerative diseases and may serve as promising therapeutic target. Cuenda, A. p38 MAP-Kinases pathway regulation, function, and role in human diseases. Biochimica et Biophysica Acta. 2007; 1773: 1358-1375; Thalhamer et al., Rheumatology 2008; 47:409-414; Roux, P. ERK and p38 MAPK-Activated Protein Kinases: a Family of Protein Kinases with Diverse Biological Functions. Microbiology and Molecular Biology Reviews. June, 2004; 320-344 hereby fully incorporated by reference in its entirety for all purposes.

Src Family Kinases:

Src is the most widely studied member of the largest family of nonreceptor protein tyrosine kinases, known as the Src family kinases (SFKs). Other SFK members include Lyn, Fyn, Lck, Hck, Fgr, Blk, Yrk, and Yes. The Src kinases can be grouped into two sub-categories, those that are ubiquitously expressed (Src, Fyn, and Yes), and those which are found primarily in hematopoietic cells (Lyn, Lck, Hck, Blk, Fgr). (Benati, D. Src Family Kinases as Potential Therapeutic Targets for Malignancies and Immunological Disorders. Current Medicinal Chemistry. 2008; 15: 1154-1165) SFKs are key messengers in many cellular pathways, including those involved in regulating proliferation, differentiation, survival, motility, and angiogenesis. The activity of SFKs is highly regulated intramolecularly by interactions between the SH2 and SH3 domains and intermolecularly by association with cytoplasmic molecules. This latter activation may be mediated by focal adhesion kinase (FAK) or its molecular partner Crk-associated substrate (CAS), which plays a prominent role in integrin signaling, and by ligand activation of cell surface receptors, e.g. epidermal growth factor receptor (EGFR). These interactions disrupt intramolecular interactions within Src, leading to an open conformation that enables the protein to interact with potential substrates and downstream signaling molecules. Src can also be activated by dephosphorylation of tyrosine residue Y530. Maximal Src activation requires the autophosphorylation of tyrosine residue Y419 (in the human protein) present within the catalytic domain. Elevated Src activity may be caused by increased transcription or by deregulation due to overexpression of upstream growth factor receptors such as EGFR, HER2, platelet-derived growth factor receptor (PDGFR), fibroblast growth factor receptor (FGFR), vascular endothelial growth factor receptor, ephrins, integrin, or FAK. Alternatively, some human tumors show reduced expression of the negative Src regulator, Csk. Increased levels, increased activity, and genetic abnormalities of Src kinases have been implicated in both solid tumor development and leukemias. Ingley, E. Src family kinases: Regulation of their activities, levels and identification of new pathways. Biochimica et Biophysica Acta. 2008; 1784 56-65, hereby fully incorporated by reference in its entirety for all purposes. Benati and Baldari., Curr Med Chem. 2008; 15(12):1154-65, Finn (2008) Ann Oncol. May 16, hereby fully incorporated by reference in its entirety for all purposes.

Janus Kinase (JAK)/Signal Transducers and Activators of Transcription (STAT) Pathway.

The JAK/STAT pathway plays a crucial role in mediating the signals from a diverse spectrum of cytokine receptors, growth factor receptors, and G-protein-coupled receptors. Signal transducers and activators of transcription (STAT) proteins play a crucial role in mediating the signals from a diverse spectrum of cytokine receptors growth factor receptors, and G-protein-coupled receptors. STAT directly links cytokine receptor stimulation to gene transcription by acting as both a cytosolic messenger and nuclear transcription factor. In the Janus Kinase (JAK)-STAT pathway, receptor dimerization by ligand binding results in JAK family kinase (JFK) activation and subsequent tyrosine phosphorylation of the receptor, which leads to the recruitment of STAT through the SH2 domain, and the phosphorylation of conserved tyrosine residue. Tyrosine phosphorylated STAT forms a dimer, translocates to the nucleus, and binds to specific DNA elements to activate target gene transcription, which leads to the regulation of cellular proliferation, differentiation, and apoptosis. The entire process is tightly regulated at multiple levels by protein tyrosine phosphatases, suppressors of cytokine signaling and protein inhibitors of activated STAT. In mammals seven members of the STAT family (STAT1, STAT2, STAT3, STAT4, STAT5a, STAT5b and STAT6) have been identified. JAKs contain two symmetrical kinase-like domains; the C-terminal JAK homology 1 (JH1) domain possesses tyrosine kinase function while the immediately adjacent JH2 domain is enzymatically inert but is believed to regulate the activity of JH1. There are four JAK family members: JAK1, JAK2, JAK3 and tyrosine kinase 2 (Tyk2). Expression is ubiquitous for JAK1, JAK2 and TYK2 but restricted to hematopoietic cells for JAK3. Mutations in JAK proteins have been described for several myeloid malignancies. Specific examples include but are not limited to: Somatic JAK3 (e.g. JAK3A572V, JAK3V722I, JAK3P132T) and fusion JAK2 (e.g. ETV6-JAK2, PCM1-JAK2, BCR-JAK2) mutations have respectively been described in acute megakaryocytic leukemia and acute leukemia/chronic myeloid malignancies, JAK2 (V617F, JAK2 exon 12 mutations) and MPL MPLW515L/K/S, MPLS505N) mutations associated with myeloproliferative disorders and myeloproliferative neoplasms. JAK2 mutations, primarily JAK2V617F, are invariably associated with polycythemia vera (PV). This mutation also occurs in the majority of patients with essential thrombocythemia (ET) or primary myelofibrosis (PMF) (Tefferi n., Leukemia & Lymphoma, March 2008; 49(3): 388-397). STATs can be activated in a JAK-independent manner by src family kinase members and by oncogenic FLt3 ligand-ITD (Hayakawa and Naoe, Ann NY Acad Sci. 2006 November; 1086:213-22; Choudhary et al. Activation mechanisms of STAT5 by oncogenic FLt3 ligand-ITD. Blood (2007) vol. 110 (1) pp. 370-4). Although mutations of STATs have not been described in human tumors, the activity of several members of the family, such as STAT1, STAT3 and STAT5, is dysregulated in a variety of human tumors and leukemias. STAT3 and STAT5 acquire oncogenic potential through constitutive phosphorylation on tyrosine, and their activity has been shown to be required to sustain a transformed phenotype. This was shown in lung cancer where tyrosine phosphorylation of STAT3 was JAK-independent and mediated by EGF receptor activated through mutation and Src. (Alvarez et al., Cancer Research, Cancer Res 2006; 66) STAT5 phosphorylation was also shown to be required for the long-term maintenance of leukemic stem cells. (Schepers et al. STAT5 is required for long-term maintenance of normal and leukemic human stem/progenitor cells. Blood (2007) vol. 110 (8) pp. 2880-2888) In contrast to STAT3 and STAT5, STAT1 negatively regulates cell proliferation and angiogenesis and thereby inhibits tumor formation. Consistent with its tumor suppressive properties, STAT1 and its downstream targets have been shown to be reduced in a variety of human tumors (Rawlings, J. The JAK/STAT signaling pathway. J of Cell Science. 2004; 117 (8):1281-1283, hereby fully incorporated by reference in its entirety for all purposes).

### DNA Damage Response/Repair and Apoptosis Pathways

The response to DNA damage is a protective measure taken by cells to prevent or delay genetic instability and tumorigenesis. It allows cells to undergo cell cycle arrest and gives them an opportunity to either: repair the broken DNA and resume passage through the cell cycle or, if the breakage is irreparable, trigger senescence or an apoptotic program leading to cell death.

Several protein complexes are positioned at strategic points within the DNA damage response pathway and act as sensors, transducers or effectors of DNA damage. Depending on the nature of DNA damage for example; double stranded breaks, single strand breaks, single base alterations due to alkylation, oxidation etc, there is an assembly of specific DNA damage sensor protein complexes in which activated ataxia telangiectasia mutated (ATM) and ATM- and Rad3 related (ATR) kinases phosphorylate and subsequently activate the checkpoint kinases Chk1 and Chk2. Both of these DNA-signal transducer kinases amplify the damage response by phosphorylating a multitude of substrates. Both checkpoint kinases have overlapping and distinct roles in orchestrating the cell's response to DNA damage.

Maximal kinase activation of Chk2 involves phosphorylation and homo-dimerization with ATM-mediated phosphorylation of T68 on Chk2 as a preliminary event. This in turn activates the DNA repair. As mentioned above, in order for DNA repair to proceed, there can be a delay in the cell cycle. Chk2 seems to have a role at the G1/S and G2/M junctures and may have overlapping functions with Chk1. There are multiple ways in which Chk1 and Chk2 mediate cell cycle suspension. In one mechanism Chk2 phosphorylates the CDC25A and CDC25C phosphatases resulting in their removal from the nucleus either by proteosomal degradation or by sequestration in the cytoplasm by 14-3-3. These phosphatases are no longer able to act on their nuclear CDK substrates. If DNA repair is successful cell cycle progression is resumed.

When DNA repair is no longer possible the cell undergoes apoptosis with participation from Chk2 in p53 independent and dependent pathways. Chk2 substrates that operate in a p53-independent manner include the E2F1 transcription factor, the tumor suppressor promyelocytic leukemia (PML) and the polo-like kinases 1 and 3 (PLK1 and PLK3). E2F1 drives the expression of a number of apoptotic genes including caspases 3, 7, 8 and 9 as well as the pro-apoptotic Bcl-2 related proteins (Bim, Noxa, PUMA).

In its response to DNA damage, the p53 activates the transcription of a program of genes that regulate DNA repair, cell cycle arrest, senescence and apoptosis. The overall functions of p53 are to preserve fidelity in DNA replication such that when cell division occurs tumorigenic potential can be avoided. In such a role, p53 is described as “The Guardian of the Genome.” The diverse alarm signals that impinge on p53 result in a rapid increase in its levels through a variety of post translational modifications. Worthy of mention is the phosphorylation of amino acid residues within the amino terminal portion of p53 such that p53 is no longer under the regulation of Mdm2. The responsible kinases are ATM, Chk1 and Chk2. The subsequent stabilization of p53 permits it to transcriptionally regulate multiple pro-apoptotic members of the Bcl-2 family, including Bax, Bid, Puma, and Noxa (discussion below).

The series of events that are mediated by p53 to promote apoptosis including DNA damage, anoxia and imbalances in growth-promoting signals are sometimes termed the ‘intrinsic apoptotic” program since the signals triggering it originate within the cell. An alternate route of activating the apoptotic pathway can occur from the outside of the cell mediated by the binding of ligands to transmembrane death receptors. This extrinsic or receptor mediated apoptotic program acting through their receptor death domains eventually converges on the intrinsic, mitochondrial apoptotic pathway as discussed below

Key regulators of apoptosis are proteins of the Bcl-2 family. The founding member, the Bcl-2 proto-oncogene was first identified at the chromosomal breakpoint of t(14:18) bearing human follicular B cell lymphoma. Unexpectedly, expression of Bcl-2 was proved to block rather than promote cell death following multiple pathological and physiological stimuli The Bcl-2 family has at least 20 members which are key regulators of apoptosis, functioning to control mitochondrial permeability as well as the release of proteins important in the apoptotic program. The ratio of anti- to pro-apoptotic molecules such as Bcl-2/Bax constitutes a rheostat that sets the threshold of susceptibility to apoptosis for the intrinsic pathway, which utilizes organelles such as the mitochondrion to amplify death signals. The family can be divided into 3 subclasses based on structure and impact on apoptosis. Family members of subclass 1 including Bcl-2, Bcl-X_{L} and Mcl-1 are characterized by the presence of 4 Bcl-2 homology domains (BH1, BH2, BH3 and BH4) and are anti-apoptotic. The structure of the second subclass members is marked for containing 3 BH domains and family members such as Bax and Bak possess pro-apoptotic activities. The third subclass, termed the BH3-only proteins include Noxa, Puma, Bid, Bad and Bim. They function to promote apoptosis either by activating the pro-apoptotic members of group 2 or by inhibiting the anti-apoptotic members of subclass 1.

The role of mitochondria in the apoptotic process was clarified as involving an apoptotic stimulus resulting in depolarization of the outer mitochondrial membrane leading to a leak of cytochrome C into the cytoplasm. Association of Cytoplasmic cytochrome C molecules with adaptor apoptotic protease activating factor (APAF) forms a structure called the apoptosome which can activate enzymatically latent procaspase 9 into a cleaved activated form. Caspase 9 is one member of a family of cysteine aspartyl-specific proteases; genes encoding 11 of these proteases have been mapped in the human genome. Activated caspase 9, classified as an intiator caspase, then cleaves procaspase 3 which cleaves more downstream procaspases, classified as executioner caspases, resulting in an amplification cascade that promotes cleavage of death substrates including poly(ADP-ribose) polymerase 1 (PARP). The cleavage of PARP produces 2 fragments both of which have a role in apoptosis. A further level of apoptotic regulation is provided by smac/Diablo, a mitochondrial protein that inactivates a group of anti-apoptotic proteins termed inhibitors of apoptosis (IAPs) IAPs operate to block caspase activity in 2 ways; they bind directly to and inhibit caspase activity and in certain cases they can mark caspases for ubiquitination and degradation.

Members of the caspase gene family (cysteine proteases with aspartate specificity) play significant roles in both inflammation and apoptosis. Caspases exhibit catalytic and substrate recognition motifs that have been highly conserved. These characteristic amino acid sequences allow caspases to interact with both positive and negative regulators of their activity. The substrate preferences or specificities of individual caspases have been exploited for the development of peptides that successfully compete for caspase binding. In addition to their distinctive aspartate cleavage sites at the P1 position, the catalytic domains of the caspases require at least four amino acids to the left of the cleavage site with P4 as the prominent specificity-determining residue. WEHD, VDVAD, and DEVD are examples of peptides that preferentially bind caspase-1, caspase-2 and caspase-3, respectively. It is possible to generate reversible or irreversible inhibitors of caspase activation by coupling caspase-specific peptides to certain aldehyde, nitrile or ketone compounds. These caspase inhibitors can successfully inhibit the induction of apoptosis in various tumor cell lines as well as normal cells. Fluoromethyl ketone (FMK)-derivatized peptides act as effective irreversible inhibitors with no added cytotoxic effects. Inhibitors synthesized with a benzyloxycarbonyl group (also known as BOC or Z) at the N-terminus and O-methyl side chains exhibit enhanced cellular permeability thus facilitating their use in both in vitro cell culture as well as in vivo animal studies. Benzyloxycarbonyl-Val-Ala-Asp (OMe) fluoromethylketone (ZVAD) is a caspase inhibitor. See Misaghi, et al., z-VAD-fmk inhibits peptide:N-glycanase and may result in ER stress.

The balance of pro- and anti-apoptotic proteins is tightly regulated under normal physiological conditions. Tipping of this balance either way results in disease. An oncogenic outcome results from the inability of tumor cells to undergo apoptosis and this can be caused by over-expression of anti-apoptotic proteins or reduced expression or activity of pro-apoptotic protein

In some embodiments, the status of an activatable element within an apoptosis pathway is determined. In some embodiments, the activatable element within the apoptosis pathway is selected from the group consisting of Cleaved PARP (PARP+), Cleaved Caspase 8, and Cytoplasmic Cytochrome C.

In some embodiments, the status of an activatable element within a DNA damage pathway is determined. In some embodiments, the activatable element within a DNA damage pathway is selected from the group consisting of p-CHk1, p-Chk-2, p-ATM, p-p53, p-ATR, p-21, and p-H2AX.

### Cell Cycle

The cell cycle, or cell-division cycle, is the series of events that take place in a cell leading to its division and duplication (replication). The cell cycle consists of five distinct phases: G1 phase, S phase (synthesis), G2 phase (collectively known as interphase) and M phase (mitosis). M phase is itself composed of two tightly coupled processes: mitosis, in which the cell's chromosomes are divided between the two daughter cells, and cytokinesis, in which the cell's cytoplasm divides forming distinct cells. Activation of each phase is dependent on the proper progression and completion of the previous one. Cells that have temporarily or reversibly stopped dividing are said to have entered a state of quiescence called G0 phase.

Regulation of the cell cycle involves processes crucial to the survival of a cell, including the detection and repair of genetic damage as well as the prevention of uncontrolled cell division. The molecular events that control the cell cycle are ordered and directional; that is, each process occurs in a sequential fashion and it is impossible to “reverse” the cycle.

Two key classes of regulatory molecules, cyclins and cyclin-dependent kinases (CDKs), determine a cell's progress through the cell cycle. Many of the genes encoding cyclins and CDKs are conserved among all eukaryotes, but in general more complex organisms have more elaborate cell cycle control systems that incorporate more individual components. Many of the relevant genes were first identified by studying yeast, especially Saccharomyces cerevisiae genetic nomenclature in yeast dubs many these genes cdc (for “cell division cycle”) followed by an identifying number, e.g., cdc25.

Cyclins form the regulatory subunits and CDKs the catalytic subunits of an activated heterodimer; cyclins have no catalytic activity and CDKs are inactive in the absence of a partner cyclin. When activated by a bound cyclin, CDKs perform a common biochemical reaction called phosphorylation that activates or inactivates target proteins to orchestrate coordinated entry into the next phase of the cell cycle. Different cyclin-CDK combinations determine the downstream proteins targeted. CDKs are constitutively expressed in cells whereas cyclins are synthesised at specific stages of the cell cycle, in response to various molecular signals.

Upon receiving a pro-mitotic extracellular signal, G1 cyclin-CDK complexes become active to prepare the cell for S phase, promoting the expression of transcription factors that in turn promote the expression of S cyclins and of enzymes required for DNA replication. The G1 cyclin-CDK complexes also promote the degradation of molecules that function as S phase inhibitors by targeting them for ubiquitination. Once a protein has been ubiquitinated, it is targeted for proteolytic degradation by the proteasome. Active S cyclin-CDK complexes phosphorylate proteins that make up the pre-replication complexes assembled during G1 phase on DNA replication origins. The phosphorylation serves two purposes: to activate each already-assembled pre-replication complex, and to prevent new complexes from forming. This ensures that every portion of the cell's genome will be replicated once and only once. The reason for prevention of gaps in replication is fairly clear, because daughter cells that are missing all or part of crucial genes will die. However, for reasons related to gene copy number effects, possession of extra copies of certain genes would also prove deleterious to the daughter cells.

Mitotic cyclin-CDK complexes, which are synthesized but inactivated during S and G2 phases, promote the initiation of mitosis by stimulating downstream proteins involved in chromosome condensation and mitotic spindle assembly. A critical complex activated during this process is an ubiquitin ligase known as the anaphase-promoting complex (APC), which promotes degradation of structural proteins associated with the chromosomal kinetochore. APC also targets the mitotic cyclins for degradation, ensuring that telophase and cytokinesis can proceed. Interphase: Interphase generally lasts at least 12 to 24 hours in mammalian tissue. During this period, the cell is constantly synthesizing RNA, producing protein and growing in size. By studying molecular events in cells, scientists have determined that interphase can be divided into 4 steps: Gap 0 (G0), Gap 1 (G1), S (synthesis) phase, Gap 2 (G2).

Cyclin D is the first cyclin produced in the cell cycle, in response to extracellular signals (e.g. growth factors). Cyclin D binds to existing CDK4, forming the active cyclin D-CDK4 complex. Cyclin D-CDK4 complex in turn phosphorylates the retinoblastoma susceptibility protein (Rb). The hyperphosphorylated Rb dissociates from the E2F/DP1/Rb complex (which was bound to the E2F responsive genes, effectively “blocking” them from transcription), activating E2F. Activation of E2F results in transcription of various genes like cyclin E, cyclin A, DNA polymerase, thymidine kinase, etc. Cyclin E thus produced binds to CDK2, forming the cyclin E-CDK2 complex, which pushes the cell from G1 to S phase (G1/S transition). Cyclin B along with cdc2 (cdc2—fission yeasts (CDK1—mammalia)) forms the cyclin B-cdc2 complex, which initiates the G2/M transition. Cyclin B-cdc2 complex activation causes breakdown of nuclear envelope and initiation of prophase, and subsequently, its deactivation causes the cell to exit mitosis.

Two families of genes, the Cip/Kip family and the INK4a/ARF (Inhibitor of Kinase 4/Alternative Reading Frame) prevent the progression of the cell cycle. Because these genes are instrumental in prevention of tumor formation, they are known as tumor suppressors.

The Cip/Kip family includes the genes p21, p27 and p57. They halt cell cycle in G1 phase, by binding to, and inactivating, cyclin-CDK complexes. p21 is a p53 response gene (which, in turn, is triggered by DNA damage eg. due to radiation). p27 is activated by Transforming Growth Factor β (TGF β), a growth inhibitor.

The INK4a/ARF family includes p16INK4a, which binds to CDK4 and arrests the cell cycle in G1 phase, and p14arf which prevents p53 degradation.

Cell cycle checkpoints are used by the cell to monitor and regulate the progress of the cell cycle. Checkpoints prevent cell cycle progression at specific points, allowing verification of necessary phase processes and repair of DNA damage. The cell cannot proceed to the next phase until checkpoint requirements have been met.

Several checkpoints are designed to ensure that damaged or incomplete DNA is not passed on to daughter cells. Two main checkpoints exist: the G1/S checkpoint and the G2/M checkpoint. G1/S transition is a rate-limiting step in the cell cycle and is also known as restriction point. An alternative model of the cell cycle response to DNA damage has also been proposed, known as the postreplication checkpoint. p53 plays an important role in triggering the control mechanisms at both G1/S and G2/M checkpoints.

### Binding Element

The term “binding element” includes any molecule, e.g., peptide, nucleic acid, small organic molecule which is capable of detecting form of an activatable element over another form of the activatable element. A “detectable binding element” as that term is used herein, encompasses a binding element, that both preferentially binds to one form of an activatable element, and whose bound form can be detected, e.g., through a label, such as a fluorescent label for flow cytometry or a mass label, also referred to as a mass tag, in mass cytometry, that produces a signal that can be detected, e.g., by a cytometer. A “detectable binding element” as that term is used herein, encompasses a detectable binding element signal whose signal can be distinguished from that of any other detectable binding element in the particular process or composition in which it is used. The signal that is detected can a quantitative value and it may be manipulated to produce other quantitative values. The values may be used to gate cells, as known in the art and as described herein. Gating may include an automatic component. Gating may include a manual component. In certain embodiments, gating includes both a manual and an automatic component; see, e.g., U.S. Patent Application No. 2013/01763618.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly.

In some embodiments, the binding element is an antibody. In some embodiment, the binding element is an activation state-specific antibody.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and posses other variations. See U.S. Ser. Nos. 12/432,720, 12/229,476, 12/460,029, and 12/910,769 for more information about antibodies as binding elements.

The antigenicity of an activated form of an activatable element can be distinguishable from the antigenicity of non-activated form of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of a moiety to an element, such as a phosphate moiety, or due to a structural change in an element, as through protein cleavage, or due to an otherwise induced conformational change in an element which causes the element to present the same sequence in an antigenically distinguishable way. Such a conformational change can cause an activated isoform of an element to present at least one epitope that is not present in a non-activated isoform, or to not present at least one epitope that is presented by a non-activated isoform of the element.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoetin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets family transcription factors, Ets-1, Ets-2, Tel, Tel2, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. In some embodiments, the protein is S6.

A binding element can be a peptide comprising a recognition structure that binds to a target structure on an activatable protein. A variety of recognition structures are well known in the art and can be made using methods known in the art, including by phage display.

A binding element can be a nucleic acid. The term “nucleic acid” include nucleic acid analogs, for example, phosphoramide, phosphorothioate, phosphorodithioate, O-methylphophoroamidite linkages, and peptide nucleic acid backbones and linkages. Other analog nucleic acids include those with positive backbones; non-ionic backbones.

### Modulators

Cells can be contacted with one or more modulators. A modulator can be, e.g., an activator, a therapeutic compound, an inhibitor or a compound capable of impacting a cellular pathway. Modulators can also take the form of environmental cues and inputs.

Modulation can be performed in a variety of environments. Cells can be exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, a sample of cells can be exposed to at least 2, 3, 4, 5, 6, 7, 8, 9, or 10 or more modulators. See U.S. patent application Ser. Nos. 12/432,239 and 12/910,769 which are incorporated by reference in their entireties. See also U.S. Pat. Nos. 7,695,926 and 7,381,535 and U.S. Pub. No. 2009/0269773.

Cells can be cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%, about 0.001% to 30%, about 0.01% to 30%, about 0.1% to 30% or 1% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, mitogens, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

The modulator can be selected from the group consisting of growth factors, mitogens, cytokines, adhesion molecules, drugs, hormones, small molecules, polynucleotides, antibodies, natural compounds, lactones, chemotherapeutic agents, immune modulators, carbohydrates, proteases, ions, reactive oxygen species, peptides, and protein fragments, either alone or in the context of cells, cells themselves, viruses, and biological and non-biological complexes (e.g. beads, plates, viral envelopes, antigen presentation molecules such as major histocompatibility complex). In some embodiments, the modulator is a physical stimuli such as heat, cold, UV radiation, and radiation. Examples of modulators, include but are not limited to Growth factors, such as Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Stromal Derived Growth Factor, (SDGF), Thrombopoietin (TPO), Transforming growth factor alpha (TGF-α), Transforming growth factor beta (TGF-β), Tumour necrosis factor-alpha (TNF-α), Vascular endothelial growth factor (VEGF), Keratin Derived Growth Factor (KGF), Wnt Signaling Pathway, placental growth factor (PlGF), [(Foetal Bovine Somatotrophin)] (FBS), IL-1—Cofactor for IL-3 and IL-6. Activates T cells, IL-2—T-cell growth factor. Stimulates IL-1 synthesis. Activates B-cells and NK cells, IL-3—Stimulates production of all non-lymphoid cells, IL-4—Growth factor for activated B cells, resting T cells, and mast cells, IL-5—Induces differentiation of activated B cells and eosinophils, IL-6—Stimulates Ig synthesis. Growth factor for plasma cells, and IL-7—Growth factor for pre-B cells. Cell motility factors, such as peptide growth factors, (e.g., EGF, PDGF, TGF-beta), substrate-adhesion molecules (e.g., fibronectin, laminin), cell adhesion molecules (CAMs), and metalloproteinases, hepatocyte growth factor (HGF) or scatter factor (SF), autocrine motility factor (AMF), and migration-stimulating factor (MSF). Other modulators include SDF-1α, IFN-α, IFN-γ, IL-10, IL-6, IL-27, G-CSF, FLT-3L, IGF-1, M-CSF, SCF, PMA, Thapsigargin, H₂O₂, Etoposide, Mylotarg, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L, and combinations thereof.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

The modulator can be a cross-linker. The cross-linker can be a molecular binding entity. In some embodiments, the molecular binding entity is a monovalent, bivalent, or multivalent is made more multivalent by attachment to a solid surface or tethered on a nanoparticle surface to increase the local valency of the epitope binding domain.

The modulator can be an inhibitor. The inhibitor can be an inhibitor of a cellular factor or a plurality of factors that participates in a cellular pathway (e.g. signaling cascade) in the cell. In some embodiments, the inhibitor is a phosphataseor a tyrosine kinase inhibitor. Examples of phosphatase inhibitors include, but are not limited to H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, 3-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenylarsine oxide, Pyrrolidine Dithiocarbamate, and Aluminium fluoride. In some embodiments, the phosphatase inhibitor is H₂O₂.

The activation level of an activatable element in a cell can be determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

A phenotypic profile of a population of cells can be determined by measuring the activation level of an activatable element when the population of cells is exposed to a plurality of modulators in separate cultures. In some embodiments, the modulators include PMA, SDF1 α, CD40L, IGF-1, IL-7, IL-6, IL-10, IL-27, IL-4, IL-2, IL-3, and/or a combination thereof. For instance a population of cells can be exposed to one or more, all or a combination of the following combination of modulators: PMA; SDF1α; CD40L; IGF-1; IL-7; IL-6; IL-10; IL-27; IL-4; IL-2; IL-3. In some embodiments, the phenotypic profile of the population of cells is used to classify the population as described herein.

### Detection

One or more activatable elements can be detected and/or quantified by any method that detects and/or quantitates the presence of the activatable element of interest. Such methods may include flow cytometry, mass cytometry, radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western, Northern, and Southern blots, PCR, nucleic acid sequencing, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters.

A cytometer can be used, for example a flow cytometer or a mass cytometer, e.g., a CyToF, may be used. Commercial instruments are available through Becton Dickinson, Beckman Coulter, and Fluidigm, among others.

When a binding element is detected through a fluorescent signal, fluorescence can be measured using a fluorimeter. In general, excitation radiation, from an excitation source having a first wavelength, passes through excitation optics. The excitation optics deliver the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The binding element may be detected by a signal from a label that is detectable by a mass spectrometer, e.g., a mass tag. An example is an Inductively Coupled Plasma Spectrometer (ICP-MS). A binding element that has been labeled with a specific element binds to one form of an activatable element. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell.

Detection using a mass spectrometer, when coupled with cytometric techniques similar to those used in flow cytometry, is referred to as “mass cytometry” herein. The technique is very similar to flow cytometry, and sample preparation can be carried out using essentially identical techniques, e.g., 96-well plates, modulation, fixing, permeabilizing, the use of antibodies as binding agents. The difference is that the antibodies are tagged with mass labels rather than fluorescent labels, and that detection is carried out by mass spectrometry. The signals from the mass spectrometer are analogous to those of a flow cytometer, i.e., a signal is generated that is proportional to the level of a particular element of the cell being investigated, such as the expression level of a surface marker, or the activation level of an activatable element. Thus, much of the data acquisition and handling is analogous to that for flow cytometry. Mass cytometry presents the potential advantage of being capable of detecting a larger number of signals that flow cytometry, e.g., the CyTof instrument (Fluidigm), can detect up to 34 parameters for a single cell, as opposed to the current maximum of 12 parameters for flow cytometers (in addition to scatter characteristics, e.g., SSC and FSC). This makes it particularly useful for many techniques of the invention, where the determination of the expression levels of numerous cell surface elements, e.g., CD and other markers useful for classifying cells, as well as IMRs, may be desired for single cells, as well as, in many cases, additional expression levels for intracellular molecules, e.g., cytokines, or activation levels of activatable proteins. For example, it may be desirable to determine expression levels of a plurality of IMRs in a single cell, e.g., at least 3, or at least 6, or even at least 10 individual IMRs, and often, in the same cell, also determine the functional status of at least one of the IMRs in the cell, which would require determining the level of at least one intracellular element after stimulation of the cell and the IMR (e.g., stimulation of the TCR and one or more IMRs in a T cell), such as the expression level of an intracellular element such as a protein, or the activation level of an intracellular activatable element such as a protein, e.g., phosphoproteins. In addition, generally it will be desirable to determine which cell population or subpopulation the cell belongs to, which usually requires the determination of levels of 1-4 or even more surface markers. As another example, in APCs and/or tumor cells, it may be desirable to determine the levels of a plurality of ligands for IMRs, i.e., the same number or even more than the number of the IMRs themselves, and/or surface markers for identification of tumor or of APC type and/or markers for intracellular pathways in the cells to determine functional status of the pathways. This presents a challenge for even the most advanced of presently available flow cytometers, but a mass cytometer can give reliable readings for a sufficient number of different channels to allow such measurements. However, in many cases, it may be found that the number of channels required for the methods and compositions of the invention, e.g., prediction of response to treatment, is such that a flow cytometer can be used as the detection instrument. As many of the steps of sample preparation and data analysis are similar or even the same for flow cytometry and mass cytometry, detection instruments and techniques are described herein for flow cytometers using, typically, fluorescent detection. However, it is understood that the same or similar techniques can be used for mass cytometry, and one of skill in the art understands the necessary adjustments required to apply the flow cytometric techniques to mass cytometry.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods described herein (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an activatable element in response to a modulator. Other flow cytometers that are commercially available include the LSR II and the Canto II both available from Becton Dickinson others are available from Attune Acoustic Cytometer (Life Technologies, Carlsbad, Calif.) and the CyTOF (DVS Sciences, Sunnyvale, Calif.). See Shapiro, Howard M., Practical Flow Cytometry, 4th Ed., John Wiley & Sons, Inc., 2003 for additional information on flow cytometers.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™ Manual, with particular reference to sections 3-11 to 3-28 and 10-1 to 10-17, which is hereby incorporated by reference in its entirety. See the patents, applications and articles referred to, and incorporated above for detection systems.

Fluorescent compounds such as Daunorubicin and Enzastaurin are problematic for flow cytometry based biological assays due to their broad fluorescence emission spectra. These compounds get trapped inside cells after fixation with agents like paraformaldehyde, and are excited by one or more of the lasers found on flow cytometers. The fluorescence emission of these compounds is often detected in multiple PMT detectors which complicates their use in multiparametric flow cytometry. A way to get around this problem is to compensate out the fluorescence emission of the compound from the PMT detectors used to measure the relevant biological markers. This is achieved using a PMT detector with a bandpass filter near the emission maximum of the fluorescent compound, and cells incubated with the compound as the compensation control when calculating a compensation matrix. The cells incubated with the fluorescent compound are fixed with paraformaldehyde, then washed and permeabilized with 100% methanol. The methanol is washed out and the cells are mixed with unlabeled fixed/permed cells to yield a compensation control consisting of a mixture of fluorescent and negative cell populations.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex manual which is hereby incorporated in its entirety.

In some embodiments, cell analysis by flow cytometry on the basis of the activation level of at least one element is combined with a determination of other flow cytometry readable outputs, such as the presence of surface markers, granularity and cell size to provide a further information on other cell qualities measurable by flow cytometry for single cells.

As will be appreciated, methods described herein also provide for the ordering of element clustering events in signal transduction. Particularly, the methods described herein allow the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

The methods described herein provide a valuable method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the present invention allows the individual evaluation of cells to allow true differences to be identified in a significant way.

When necessary cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES 1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200° C.; and the like as known in the art and according to the methods described herein.

A permeabilizing agent, for example, a methanol dispensing instrument can used to permeabilize the cells. It is important to ensure that the correct volume of methanol is being dispensed into the wells, otherwise the labeling reagents will not have access to their targets. To ensure that the appropriate amount of methanol is dispensed, the dispenser is charged beforehand with methanol or is charged with methanol either manually or automatically.

The methanol dispensing heads in the instrument can be stored with methanol or air in the dispensing channels. Air can be drawn through the dispensing heads, then an alcohol solution and then stored air dried or with methanol. Upon reuse of the instrument or any restart of the process, the dispensing heads are recharged with methanol. A bleeder valve can be used to fill up the head with the correct amount of methanol. In one embodiment, the instrument dispenser is charged by flushing several methanol washes through the dispenser head. In one embodiment, 2, 3, 4, 5, 6, washes are used to fill and clean the head.

In some embodiments, the present invention uses platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. One embodiment uses microtiter plates and reference will be made to this embodiment as a representative of those articles that can contain samples to be analyzed.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful for the present invention will be apparent to the skilled artisan. Methods to automate the analysis are shown in U.S. Ser. No. 12/606,869 which is hereby incorporated by reference in its entirety.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using a mass spectrometer, e.g., an Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell.

As will be appreciated by one of skill in the art, the instant methods and compositions find use in a variety of other assay formats in addition to cytometry analysis.

Confocal microscopy can be used for detection. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, the cells can be labeled with one or more binding elements. In some embodiments the binding elements used in connection with confocal microscopy are antibodies conjugated to fluorescent labels, however other binding elements, such as other proteins or nucleic acids are also possible.

Another detection method is an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiments, the methods described herein include the use of liquid handling components. The liquid handling systems can include robotic systems comprising any number of components. In addition, any or all of the steps outlined herein may be automated; thus, for example, the systems may be completely or partially automated. See U.S. Ser. Nos. 12/606,869 and 12/432,239.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods described herein include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices described herein. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc. See U.S. Ser. No. 12/606,869 for automated systems.

Any of the steps above can be performed by a computer program product that comprises a computer executable logic that is recorded on a computer readable medium. For example, the computer program can execute some or all of the following functions: (i) exposing reference population of cells to one or more modulators, (ii) exposing reference population of cells to one or more binding elements, (iii) detecting the activation levels of one or more activatable elements, (iv) characterizing one or more cellular pathways and/or, (v) classifying one or more cells into one or more classes based on the activation level (vi) determining cell health status of a cell, (vii) determining the percentage of viable cells in a sample; (viii) determining the percentage of healthy cells in a sample; (ix) determining a cell signaling profile; (x) adjusting a cell signaling profile based on the percentage of healthy cells in a sample; (xi) adjusting a cell signaling profile for an individual cell based on the health of the cell; (xii) excluding or including a cell or population of cells in a cell signaling analysis based on the health of the cell or population of cells; (xiii) assaying for one or more cell health markers; and/or (xiv) assaying for one or more apoptosis and/or necrosis markers.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of determining the status of an individual by accessing data that reflects the activation level of one or more activatable elements in the reference population of cells.

### Data Analysis

Advances in flow cytometry have enabled the individual cell enumeration of up to thirteen simultaneous parameters and are moving towards the study of genomic and proteomic data subsets; in mass cytometry, the number is even higher. Likewise, advances in other techniques (e.g. microarrays, mass cytometry) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. Methods for the analysis of multiple parameters are well known in the art. See U.S. Ser. Nos. 11/338,957, 12/910,769, 12/293,081, 12/538,643, 12/501,274 12/606,869 and PCT/2011/48332 for more information on analysis. See U.S. Ser. No. 12/501,295 for gating analysis.

The data, e.g., fluorescent intensity raw data, from the detector, such as a flow cytometer, is subject to processing using metrics outlined below. After treatment with the metrics, the data can be fed to a model, such as machine learning, data mining, classification, or regression to provide a model for an outcome. There is also a selection of models to produce an outcome, which can be a prediction or a prognosis.

The data can also be processed by using characteristics of cell health and cell maturity. Information on how to use cell health to analyze cells is shown in U.S. Ser. No. 61/436,534 and PCT/US2011/01565 which are incorporated by reference in their entireties. Restricting the analysis to cells that are not in active apoptosis can provide a more useful answer. For example, in one embodiment, a method is provided to analyze cells comprising obtaining cells, determining if the cell is undergoing apoptosis and then excluding cells from a final analysis that are undergoing apoptosis. One way to determine if a cell is undergoing apoptosis is by measuring the intracellular level of one or more activatable elements related to cell health such as cleaved PARP, MCL-1, or other compounds whose activation state or activation level correlate to a level of apoptosis within single cells.

Indicators for cell health can include molecules and activatable elements within molecules associated with apoptosis, necrosis, and/or autophagy, including but not limited to caspases, caspase cleavage products such as dye substrates, cleaved PARP, cleaved cytokeratin 18, cleaved caspase, cleaved caspase 3, cytochrome C, apoptosis inducing factor (AIF), Inhibitor of Apoptosis (IAP) family members, as well as other molecules such as Bcl-2 family members including anti-apoptotic proteins (MCL-1, BCL-2, BCL-XL), BH3-only apoptotic sensitizers (PUMA, NOXA, Bim, Bad), and pro-apoptotic proteins (Bad, Bax) (see below), p53, c-myc proto-oncogene, APO-1/Fas/CD95, growth stimulating genes, or tumor suppressor genes, mitochondrial membrane dyes, Annexin-V, 7-AAD, Amine Aqua, trypan blue, propidium iodide or other viability dyes.

Another general method for analyzing cells takes into account the maturity level of the cells. In one embodiment, cells that are immature (blasts) are included in the analysis and mature cells are not included. In another embodiment, the analysis can include all the patient's cells if they go above a certain threshold for the entire sample, for example, a call will be made on the basis of the entire sample. For example, samples having greater than 50, 60, 65, 70, 75, 80, 85, 90, or 95% immature cells can be classified as immature as a whole. In another embodiment, only those specific cells which are classified as immature are included in the analysis, irrespective of the total number of immature cells, for example, only those cells that are classified as immature will be part of the analysis for each sample. Or, a combination of the two methods could be employed, such as the counting of individual immature cells for samples that exceed a threshold related to cell maturity.

The metrics that are employed can relate to absolute cell counts, signal, e.g., fluorescence, intensity, frequencies of cellular populations (univariate and bivariate), relative signal, e.g., fluorescence, readouts (such as signal above background, etc.), and measurements describing relative shifts in cellular populations. In one embodiment, raw intensity data is corrected for variances in the instrument. Then the biological effect can be measured, such as measuring how much signaling is going on using the basal, fold, total and delta metrics. Also, a user can look at the number of cells that show signaling using the Mann Whitney model below.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g. tumor cells). See also U.S. Ser. No. 12/501,295 for visualization tools.

For example, the “basal” metric is calculated by measuring the autofluorescence of a cell that has not been stimulated with a modulator or stained with a labeled antibody. The “total phospho” metric is calculated by measuring the autofluorescence of a cell that has been stimulated with a modulator and stained with a labeled antibody. The “fold change” metric is the measurement of the total phospho metric divided by the basal metric. The quadrant frequency metric is the frequency of cells in each quadrant of the contour plot

A user may also analyze multimodal distributions to separate cell populations. In some embodiments, metrics can be used for analyzing bimodal and spread distribution. In some embodiments, a Mann-Whitney U Metric is used.

In some embodiments, metrics that calculate the percent of positive above unstained and metrics that calculate MFI of positive over untreated stained can be used.

A user can create other metrics for measuring the negative signal. For example, a user may analyze a “gated unstained” or ungated unstained autofluorescence population as the negative signal for calculations such as “basal” and “total”. This is a population that has been stained with surface markers such as CD33 and CD45 to gate the desired population, but is unstained for the fluorescent parameters to be quantitatively evaluated for node determination. However, every antibody has some degree of nonspecific association or “stickyness” which is not taken into account by just comparing fluorescent antibody binding to the autofluorescence. To obtain a more accurate “negative signal”, the user may stain cells with isotype-matched control antibodies. In addition to the normal fluorescent antibodies, in one embodiment, (phospho) or non phosphopeptides which the antibodies should recognize will take away the antibody's epitope specific signal by blocking its antigen binding site allowing this “bound” antibody to be used for evaluation of non-specific binding. In another embodiment, a user may block with unlabeled antibodies. This method uses the same antibody clones of interest, but uses a version that lacks the conjugated fluorophore. The goal is to use an excess of unlabeled antibody with the labeled version. In another embodiment, a user may block other high protein concentration solutions including, but not limited to fetal bovine serum, and normal serum of the species in which the antibodies were made, i.e. using normal mouse serum in a stain with mouse antibodies. (It is preferred to work with primary conjugated antibodies and not with stains requiring secondary antibodies because the secondary antibody will recognize the blocking serum). In another embodiment, a user may treat fixed cells with phosphatases to enzymatically remove phosphates, then stain.

In alternative embodiments, there are other ways of analyzing data, such as third color analysis (3D plots), which can be similar to Cytobank 2D, plus third D in color.

There are different ways to compare the distribution of X versus the distribution of Y. Examples are described below, such as Mann Whitney, U_{U}, fold change, and percent positive. There are also different biological processes to measure using the above metrics, such as modulated to unmodulated states, basal to autofluorescence, different cell types such as leukemic cell to lymphocytes, and mature as compared to immature cells.

One embodiment of the present invention is software to examine the correlations among phosphorylation or expression levels of pairs of proteins in response to stimulus or modulation. The software examines all pairs of proteins for which phosphorylation and/or expression was measured in an experiment. The total phosho metric (sometimes called “FoldAF”) is used to represent the phosphorylation or expression data for each protein; this data is used either on linear scale or log 2 scale.

For each protein pair under each experimental condition (unstimulated, stimulated, or treated with drug/modulator), the Pearson correlation coefficient and linear regression line fit are computed. The Pearson correlation coefficients for samples representing responding and non-responding patients are calculated separately for each group and compared to the unperturbed (unstimulated) data. The following additional metrics are derived:
1. Delta CRNR unstim: the difference between Pearson correlation coefficients for each protein pair for the responding patients and for the non-responding patients in the basal or unstimulated state.
2. Delta CRNR stim: the difference between Pearson correlation coefficients for each protein pair for the responding patients and for the non-responding patients in the stimulated or treated state.
3. DeltaDelta CRNR: the difference between Delta CRNRstim and Delta CRNRunstim.

The correlation coefficients, line fit parameters (R, p-value, and slope), and the three derived parameters described above are computed for each protein-protein pair. Protein-protein pairs are identified for closer analysis by the following criteria:
1. Large shifts in correlations within patient classes as denoted by large positive or negative values (top and bottom quartile or 10^{th} and 90^{th} percentile) of the DeltaDelta CRNR parameter.
2. Large positive or negative (top and bottom quartile or 10^{th} and 90^{th} percentile) Pearson correlation for at least one patient group in either unstimulated or stimulated/treated condition.
3. Significant line fit (p-value <=0.05 for linear regression) for at least one patient group in either unstimulated or stimulated/treated condition.

All pair data is plotted as a scatter plot with axes representing phosphorylation or expression level of a protein. Data for each sample (or patient) is plotted with color indicating whether the sample represents a responder (generally blue) or non-responder (generally red). Further line fits for responders, non-responders and all data are also represented on this graph, with significant line fits (p-value <=0.05 in linear regression) represented by solid lines and other fits represented by dashed line, enabling rapid visual identification of significant fits. Each graph is annotated with the Pearson correlation coefficient and linear regression parameters for the individual classes and for the data as a whole. The resulting plots are saved in PNG format to a single directory for browsing using Picassa. Other visualization software can also be used.

In some embodiments a Mann Whitney statistical model is used for describing relative shifts in cellular populations. A Mann Whitney U test or Mann Whitney Wilcoxon (MWW) test is a non parametric statistical hypothesis test for assessing whether two independent samples of observations have equally large values. See Wikipedia at http(colon)(slashslash)en.wikipedia.org(slash)wiki/Mann%E2%80%93Whitney_U. The U metric may be more reproducible in some situations than Fold Change in some applications.

One example metric is Uᵤ. The Uᵤ is a measure of the proportion of cells that have an increase (or decrease) in protein levels upon modulation from the basal state. It is computed by dividing the scaled Mann-Whitney U statistic (http(colonslashslash)en.wikipedia.org(slash)wiki/Mann%E2%80%93Whitney_U) by the number of cells in the basal and the modulated populations. The cells in the two populations are ranked by the intensity values, only these ranks are then used to compute the statistic. As a result the metric is less sensitive to the absolute intensity values and depends only on relative shift between the two populations. The metric is bound between 0.0 and 1.0. A value of 0.5 would imply no shift in protein levels from the basal state, a value greater than 0.5 would imply an induction of signaling (i.e. increase in protein levels) and value less than 0.5 would imply an inhibition of signaling (i.e. decrease in protein levels).

$${U}_{u} = \frac{{R}_{m} - {n}_{m} ( {n}_{m} + 1 ) / 2}{{n}_{m} {n}_{u}}$$

Modulated (m) and modulated (u) populations are being compared
Rₘ=Sum of the ranks modulated population
nₘ=number of cells in the modulated population
nᵤ=number of cells in the unmodulated population

Uᵢ is another value that is the same as Uᵤ except that the isotype control is used as the reference instead of the unmodulated well.

TABLE 2
Examples of metrics.

| | | | Description and |
|---|---|---|---|
| Metric Class | Metric | Formal mathematics | Common usage |
| Absolute cell counts | Percent Recovery | $\frac{\begin{matrix}\# cells observed \\ in a sample\end{matrix}}{\begin{matrix}\# cells reported \\ in sample vial\end{matrix}}$ | Summary statistic describing the fraction of the cells that are observed after thawing and ficoll processing of cryopreserved cells |
| | Percent Viability | $\frac{\# cells Aqua negative}{total \# cells}$ | Summary statistic describing the fraction of the living cells that are |
| | | | observed from a given |
| | | | vial of samples. |
| | Percent Healthy | $\frac{\begin{matrix}\# cells Aqua negative and \\ cPARP negative\end{matrix}}{total \# cells}$ | Summary statistic describing the fraction of the living non-Apoptotic cells that are observed |
| | | | from a given vial of |
| | | | samples. |
| | Myeloid Percent Healthy | $\frac{\begin{matrix}\# cells Aqua negative \\ and cPARP negative \\ Myeloid Cells\end{matrix}}{total \# cells}$ | Summary statistic describing the fraction of the living non-Apoptotic cells that are observed from a given vial of samples. |
| Fluorescence | MFI | | A summary statistic |
| Intensity | (Median | | (median) of the non- |
| Metrics | Fluorescence | | calibrated intensity of |
| | Intensity) | | particular fluorescence |
| | | | readouts |
| | ERF | | Used to describe the |
| | (Equivalent | | fluorescence intensity |
| | Reference | | readout as calibrated for |
| | Fluorescence) | | the specific instrument |
| | | | on the specific date of |
| | | | usage. Can be applied at |
| | | | the single cell level or to |
| | | | bulk properties of |
| | | | cellular populations. |
| | | | See U.S. Pat. No. |
| | | | 8,187,885. |
| Frequencies of cellular populations- univariate | Percent of Cells | $\frac{\begin{matrix}Number cells \\ of interest\end{matrix}}{\begin{matrix}Number cells \\ Total population\end{matrix}}$ | Describes the fraction of cells of a given type relative to the population. Can be defined as a one- dimensional or 2- dimensional region or |
| | | | gate |
| | Percentage Positve | $\frac{\# cells > Cutoff}{\begin{matrix}Number cells \\ Total population\end{matrix}}$ | Describes the portion of cells above a given threshold (I.e. a control antibody) of single |
| | | | assay readout |
| Frequencies of cellular populations- bivariate | Quadrant gate “Quad” | $\frac{\begin{matrix}Number cells \\ of interest \\ in each quadrant\end{matrix}}{\begin{matrix}Number cells \\ Total population\end{matrix}}$ | Quantitative measure of the percentage of cells in each one of four regions of interest. |
| Diff | Unmodulated | log₂(MFI_{unmodulated} − | Describes the magnitude |
| | | MFI_{autofluorescence}) | of the activation levels |
| | | | of signaling in the |
| | | | resting, unmodulated |
| | | | state. This metric |
| | | | accounts for background |
| | | | autofluorescence. |
| Fold | Unmodulated | ${log}_{2} \frac{{ERF}_{unmodulated}}{{ERF}_{autofluorescence}}$ | Describes the magnitude of the activation levels of signaling in the |
| | | | resting, unmodulated |
| | | | state. This metric is |
| | | | corrected to |
| | | | accommodate the |
| | | | background |
| | | | autofluorescence. |
| | Modulated | ${log}_{2} \frac{{ERF}_{modulated}}{{ERF}_{unmodulated}}$ | Describes the magnitude of the inducibility or responsiveness of a |
| | | | protein or a signaling |
| | | | pathway activation |
| | | | response to modulation. |
| | | | This metric is always |
| | | | calculated relative to |
| | | | the unmodulated level |
| | | | of activation. Because |
| | | | unmodulated and |
| | | | modulated states are |
| | | | typically measured |
| | | | on the same plate |
| | | | several factors such as |
| | | | autofluorescence, batch |
| | | | effects, etc. are implicitly |
| | | | corrected for in this |
| | | | calculation. |
| | Total | ${log}_{2} \frac{{ERF}_{modulated}}{{ERF}_{autofluorescence}}$ | Used to assess the magnitude of total activated protein. This |
| | | | metric incorporates both |
| | | | unmodulated and |
| | | | induced pathway |
| | | | activation. |
| | Inhibited | ${log}_{2} \frac{{ERF}_{modulated + inhibited}}{{ERF}_{modulated}}$ | Used to assess the magnitude of inhibition of modulated signaling. |
| | | | This metric incorporates |
| | | | both basal and induced |
| | | | pathway activation. |
| | Relative Protein Expression | ${log}_{2} \frac{{ERF}_{Expression Marker}}{{ERF}_{isotype control}}$ | Used to measure the amount of surface expression of a particular |
| | “Rel | | protein. In this case, |
| | Expression” | | the metric is always |
| | | | caluculated relative to |
| | | | the background level of |
| | | | an isotype control. |
| Mann- Whitney U Metrics | Uₐ | $\frac{{R}_{u} - {n}_{u} ( {n}_{u} + 1 ) / 2}{{n}_{u} {n}_{a}}$ | This is a rank-based metric. It is used to describe the shift in a |
| | | Unmodulated (u) and | population or change in |
| | | autofluorescence (a) | proportion of cells in an |
| | | populations are being | unmodulated state |
| | | compared. | relative to the population |
| | | Rᵤ = Sum of the ranks | seen in the |
| | | unmodulated population | autofluorescence |
| | | nᵤ = number of cells in the | (background). All single |
| | | unmodulated population | cell events are used in |
| | | nₐ = number of cells in the | the calculation. |
| | | autofluorescence population | It is formally a scaled |
| | | | Mann-Whitney U metric |
| | | | (AUC). |
| | Uᵤ | $\frac{{R}_{m} - {n}_{m} ( {n}_{m} + 1 ) / 2}{{n}_{m} {n}_{u}}$ | This is a rank-based metric. It is used to describe the shift in a |
| | | Modulated (m) and | population or change in |
| | | unmodulated (u) | proportion of cells in a |
| | | populations are being | modulated state |
| | | compared. | relative to the population |
| | | Rₘ = Sum of the ranks | seen in the |
| | | unmodulated population | unmodulated |
| | | nₘ = number of cells in the | (basal) state. All single |
| | | modulated population | cell events are used in |
| | | nᵤ = number of cells in the | the calculation. |
| | | unmodulated population | It is formally a scaled |
| | | | Mann-Whitney U metric |
| | | | (AUC). |
| | Uᵢₘ | $\frac{{R}_{i} - {n}_{i} ( {n}_{i} + 1 ) / 2}{{n}_{i} {n}_{m}}$ | This is a rank-based metric. It is used to describe the shift in a |
| | | Modulated (m) and | population or change in |
| | | inhibited (i) | proportion of cells in an |
| | | populations are being | inhibited state |
| | | compared. | relative to the population |
| | | Rᵢ = Sum of the ranks | seen in the modulated |
| | | inhibited population | state. All single |
| | | nₘ = number of cells in the | cell events are used in |
| | | modulated population | the calculation. |
| | | nᵢ = number of cells in the | It is formally a scaled |
| | | inhibited population | Mann-Whitney |
| | | | U metric (AUC). |
| | Percent Inhibition | $\frac{\begin{matrix}Pi = 100 \times \\ {Measure}_{mod} - Measure \text{?}\end{matrix}}{{Measure}_{mod} - Measure \text{?}}$ | Used to describe the ability of a compound or other agent to modify the activity levels (assuming decreased activation) |
| | | | of a given meausre (e.g. |
| | | | MFI, ERF, etc.) |
| | Percent Inhibition of Uᵤ | $\frac{\begin{matrix}{PiU}_{u} = 100 \times \\ {U}_{{u}_{mod}} - {U}_{{u}_{mod + inhib}}\end{matrix}}{{U}_{{u}_{mod}} - 0.5}$ | Used to describe the ability of a compound or other agent to modify the activity levels (assuming decreased activation) |
| | | | using Uᵤ as the |
| | | | foundational metric. |

$\text{?} \text{indicates text missing or illegible when filed}$

Each protein pair can be further annotated by whether the proteins comprising the pair are connected in a “canonical” pathway. In the current implementation canonical pathways are defined as the pathways curated by the NCI and Nature Publishing Group. This distinction is important; however, it is likely not an exclusive way to delineate which protein pairs to examine. High correlation among proteins in a canonical pathway in a sample may indicate the pathway in that sample is “intact” or consistent with the known literature. One embodiment of the present invention identifies protein pairs that are not part of a canonical pathway with high correlation in a sample as these may indicate the non-normal or pathological signaling. This method will be used to identify stimulator/modulator-stain-stain combinations that distinguish classes of patients.

In some embodiments, nodes and/or nodes/metric combinations can be analyzed and compared across sample for their ability to distinguish among different groups (e.g., CR vs. NR patients) using classification algorithms. Any suitable classification algorithm known in the art can be used. Examples of classification algorithms that can be used include, but are not limited to, multivariate classification algorithms such as decision tree techniques: bagging, boosting, random forest, additive techniques: regression, lasso, bblrs, stepwise regression, nearest neighbors or other methods such as support vector machines.

In some embodiments, nodes and/or nodes/metric combinations can be analyzed and compared across sample for their ability to distinguish among different groups (e.g., CR vs. NR patients) using random forest algorithm. Random forest (or random forests) is an ensemble classifier that consists of many decision trees and outputs the class that is the mode of the class's output by individual trees. The algorithm for inducing a random forest was developed by Leo Breiman (Breiman, Leo (2001). “Random Forests”. Machine Learning 45 (1): 5-32. doi:10.1023/A:1010933404324) and Adele Cutler. The term came from random decision forests that was first proposed by Tin Kam Ho of Bell Labs in 1995. The method combines Breiman's “bagging” idea and the random selection of features, introduced independently by Ho (Ho, Tin (1995). “Random Decision Forest”. 3rd Int'l Conf. on Document Analysis and Recognition. pp. 278-282; Ho, Tina (1998). “The Random Subspace Method for Constructing Decision Forests”. IEEE Transactions on Pattern Analysis and Machine Intelligence 20 (8): 832-844. doi:10.1109/34.709601) and Amit and Geman (Amit, Y.; Geman, D. (1997). “Shape quantization and recognition with randomized trees”. Neural Computation 9 (7): 1545-1588. doi:10.1162/neco.1997.9.7.1545) in order to construct a collection of decision trees with controlled variation.

In some embodiments, nodes and/or nodes/metric combinations can be analyzed and compared across sample for their ability to distinguish among different groups (e.g., CR vs. NR patients) using lasso algorithm. The method of least squares is a standard approach to the approximate solution of overdetermined systems, i.e. sets of equations in which there are more equations than unknowns. “Least squares” means that the overall solution minimizes the sum of the squares of the errors made in solving every single equation. The best fit in the least-squares sense minimizes the sum of squared residuals, a residual being the difference between an observed value and the fitted value provided by a model.

In some embodiments, nodes and/or nodes/metric combinations can be analyzed and compared across sample for their ability to distinguish among different groups (e.g., CR vs. NR patients) using BBLRS model building methodology.

a. Description of the BBLRS Model Building Methodology

Production of Bootstrap Samples:

A large number of bootstrap samples are first generated with stratification by outcome status to insure that all bootstrap samples have a representative proportion of outcomes of each type. This is particularly important when the number of observations is small and the proportion of outcomes of each type is unbalanced. Stratification under such a scenario is especially critical to the composition of the out of bag (OOB) samples, since only about one-third of observations from the original sample will be included in each OOB sample.

Best Subsets Selection of Main Effects:

Best subsets selection is used to identify the combination of predictors that yields the largest score statistic among models of a given size in each bootstrap sample. Models having from 1 to 2×N/10 are typically entertained at this stage, where N is the number of observations. This is much larger than the number of predictors generally recommended when building a generalized linear prediction model (Harrell, 2001) but subsequent model building rules are applied to reduce the likelihood of over-fitting. At the conclusion of this step, there will be a “best” main effects model of each size for each bootstrap sample, though the number of unique models of each size may be considerably fewer.

Determination of the Optimal Model Size (for Main Effects):

Each of the unique “best” models of each size, identified in the previous step, are fit to each of a subset of the bootstrap samples, where the number of bootstrap samples in the subset is under the control of the user (i.e. a tuning parameter) so that the processing time required at this step can be controlled. For each of the bootstrap samples in the subset, the median SBC of the “best” models of the same size is calculated and the model size yielding the lowest median SBC in that bootstrap sample is identified. The optimal model size is then determined as the size for which the median SBC is smallest most often over the subset of bootstrap samples.

Identification of the Top Models of the Best Size:

At this stage, all previously identified “best” models of the optimal size are fit to every bootstrap sample. A number of top models are then selected as those with the highest values of the margin statistic (a measure from the logistic model of the difference in the predicted probabilities of CR, between NR patients with the highest predicted probabilities and CR patients with the lowest predicted probabilities). In order to limit the processing time required in subsequent steps, the number of top models selected is under the control of the user.

Identification of Important Two-Way Interactions:

For each of the top main effects models identified in the previous step, models are constructed on every bootstrap sample, with main effects forced into the model and with stepwise selection used to identify important two-way interactions among the set of all possible pair-wise combinations of the main effects. The nominal significance level for entry and removal of interaction terms is under the control of the user. Significance levels greater than 0.05 are often used for entry because of the low power many studies have to detect interactions and because safeguards against over-fitting are applied subsequently.

At this stage, collections of full models (main effects and possibly some two-way interactions among them) have been constructed (on the set of all bootstrap samples) for each unique set of main effects identified in the previous step. The top full models in each collection are then chosen as those constructed most frequently over all bootstrap samples, where winners are decided among tied models by the lowest mean SBC and then the highest mean AUROC. The number of full models in each collection that are advanced to the next step is under the control of the user.

Selection of the Effects in the Final Model:

Each full model advanced to this step is fit to every bootstrap sample and the median margin statistic for each model over the bootstrap samples is calculated. The model with the highest median margin statistic is selected as the final model. If there are ties, the model with the lowest mean SBC is selected.

Technically, the procedure described here results in the selection of the effects (main effects and possibly two-way interactions) to be included in the final model, but not specification of the model itself. The latter includes the effects and the specific regression coefficients associated with the intercept and each of the model effects.

Specification of the Final Model:

The effects in the final model are then fit to the complete dataset using Firth's method to apply shrinkage to the regression coefficient estimates. The model effects and their estimated regression coefficients (plus the estimate of the intercept) comprise the final model.

Another method of the present invention relates to display of information using scatter plots. Scatter plots are known in the art and are used to visually convey data for visual analysis of correlations. See U.S. Pat. No. 6,520,108. The scatter plots illustrating protein pair correlations can be annotated to convey additional information, such as one, two, or more additional parameters of data visually on a scatter plot.

Previously, scatter plots used equal size plots to denote all events. However, using the methods described herein two additional parameters can be visualized as follows. First, the diameter of the circles representing the phosphorylation or expression levels of the pair of proteins may be scaled according to another parameter. For example they may be scaled according to expression level of one or more other proteins such as transporters (if more than one protein, scaling is additive, concentric rings may be used to show individual contributions to diameter).

Second, additional shapes may be used to indicate subclasses of patients. For example they could be used to denote patients who responded to a second drug regimen or where CRp status. Another example is to show how samples or patients are stratified by another parameter (such as a different stim-stain-stain combination). Many other shapes, sizes, colors, outlines, or other distinguishing glyphs may be used to convey visual information in the scatter plot.

In this example the size of the dots is relative to the measured expression and the box around a dot indicates a NRCR patient that is a patient that became CR (Responsive) after more aggressive treatment but was initially NR (Non-Responsive). Patients without the box indicate a NR patient that stayed NR.

Applying the methods of the present invention, the Total Phospho metric for p-Akt and p-Stat1 are correlated in response to peroxide (“H₂O₂”) treatment. On log 2 scale the Pearson correlation coefficient for p-Akt and p-Stat1 in response to HOOH for samples from patients who responded to first treatment is 0.89 and the p-value for linear regression line fit is 0.0075. In contrast there appeared to be no correlation observed for p-Akt and p-Stat1 in HOOH treated samples from patients annotated as “NR” (non-responder) or “NRCR” (initial non-responder, who responded to later more intensive treatment). Further there are no significant correlations observed for these proteins in any patient class for untreated samples.

The Total phospho metric for p-Erk and p-CREB also appeared to be correlated in response to IL-3, IL-6, and IL-27 treatment in samples from non-responding patients (NR and NR-CR). When considering all data in log 2 scale the Pearson correlation coefficients for p-Erk and p-CREB in response to IL-3, IL-6, and IL-27 for samples from patients who did not respond to first treatment are 0.74, 0.76, 0.81, respectively, and the respective p-values for linear regression line fits are <0.0001, <0.0001, and <0.0001. In contrast there appeared to be no correlation observed for p-Erk and p-Creb in IL-3, IL-6, and IL-27 experiments for patients annotated as “CR”. (Not shown). Table 3(a) below shows nodes identified by a fold change metric. Table 3(b) below shows node identified by a variety of methods. In some embodiments, the nodes depicted in Tables 3(a) and 3(b) are used according to the methods described herein for classification, diagnosis, prognosis of AML or for the selection of treatment and/or predict outcome after administering a therapeutic.

In some embodiments, analyses are performed on healthy cells. In some embodiments, the health of the cells is determined by using cell markers that indicate cell health. In some embodiments, cells that are dead or undergoing apoptosis will be removed from the analysis. In some embodiments, cells are stained with apoptosis and/or cell death markers such as PARP or Aqua dyes. Cells undergoing apoptosis and/or cells that are dead can be gated out of the analysis. In other embodiments, apoptosis is monitored over time before and after treatment. For example, in some embodiments, the percentage of healthy cells can be measured at time zero and then at later time points and conditions. In some embodiments, the measurements of activatable elements are adjusted by measurements of sample quality for the individual sample, such as the percent of healthy cells present.

In some embodiments, a regression equation will be used to adjust raw node readout scores for the percentage of healthy cells at 24 hours post-thaw. In some embodiments, means and standard deviations will be used to standardize the adjusted node readout scores.

Before applying the SCNP classifier, raw node-metric signal readouts (measurements) for samples will be adjusted for the percentage of healthy cells and then standardized. The adjustment for the percentage of healthy cells and the subsequent standardization of adjusted measurements is applied separately for each of the node-metrics in the SCNP classifier.

The following formula can be used to calculate the adjusted, normalized node-metric measurement (z) for each of the node-metrics of each sample.

z=((x−(b₀+b₁×pcthealthy))−residual_mean)/residual_sd,

where x is the raw node-metric signal readout, b₀ and b₁ are the coefficients from the regression equation used to adjust for the percentage of healthy cells (pcthealthy), and residual_mean and residual_sd are the mean and standard deviation, respectively, for the adjusted signal readouts in the training set data. The values of b₀, b₁, residual_mean, and residual_sd for each node-metric are included in the embedded object below, with values of the latter two parameters stored in variables by the same name. The values of the b₀ and b₁ parameters are contained on separate records in the variable named “estimate”. The value for b₀ is contained on the record where the variable “parameter” is equal to “Intercept” and the value for b₁ is contained on the record where the variable “parameter” is equal to “percenthealthy24Hrs”. The value of pcthealthy will be obtained for each sample as part of the standard assay output. The SCNP classifier will be applied to the z values for the node-metrics to calculate the continuous SCNP classifier score and the binary induction response assignment (pNR or pCR) for each sample.

In some embodiments, the measurements of activatable elements are adjusted by measurements of sample quality for the individual cell populations or individual cells, based on markers of cell health in the cell populations or individual cells. Examples of analysis of healthy cells can be found in U.S. application Ser. No. 61/374,613 filed Aug. 18, 2010, the content of which is incorporated herein by reference in its entirety for all purposes.

### Conditions

The methods of the invention are applicable to any condition in an individual involving, indicated by, and/or arising from, in whole or in part, altered physiological status in cells. A condition involving or characterized by altered physiological status may be readily identified, for example, by determining the state of one or more activatable elements in cells from different populations, as taught herein.

In certain embodiments, the condition is cancer. The cancer may produce solid tumors or hematological tumors. Cancers that produce solid tumors include adrenal cortical cancer, anal cancer, bile duct cancer (e.g. peripheral cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). Primary cancers and metastases as well as cancers of unknown primary are included.

Cancers that produce hematological tumors include but are not limited to Non-Hodgkin Lymphoma, Hodgkin or other lymphomas, acute or chronic leukemias, and multiple myeloma. In certain embodiments, the cancer is non-B lineage derived, such as Acute myeloid leukemia (AML), Chronic Myeloid Leukemia (CML), non-B cell Acute lymphocytic leukemia (ALL), or non-B cell lymphomas. In certain embodiments, the cancer is a B-Cell or B cell lineage derived cancer. Examples of B-Cell or B cell lineage cancers include but are not limited to Chronic Lymphocytic Leukemia (CLL), B lymphocyte lineage leukemia, B lymphocyte lineage lymphoma, and Multiple Myeloma. Other conditions within the scope of the present invention include, but are not limited to, cancers such as gliomas, lung cancer, colon cancer and prostate cancer.

### Kits

In some embodiments the invention provides kits. Kits provided by the invention may comprise one or more of the state-specific binding elements described herein, such as phospho-specific antibodies. A kit may also include other reagents that are useful in the invention, such as modulators, fixatives, containers, plates, buffers, therapeutic agents, instructions, and the like.

In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tpl2, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCγ1, PLCγ 2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25,A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IkB, p65(RelA), IKKα, PK-theta, PKC, PKC-B, PKC-Q, PKC-D, CAMK, Elk, AFT, Myc, Egr-1, NFAT, ATF-2, Mdm2, p53, DNA-PK, Chk1, Chk2, ATM, ATR, B-catenin, CrkL, GSK3α, GSK3β, and FOXO. In some embodiments, the kit comprises one or more of the phospho-specific antibodies specific for the proteins selected from the group consisting of Erk, Syk, Zap70, Lck, Btk, BLNK, Cbl, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP70, Btk, BLNK, Lck, PLCγ, PLCγ 2, STAT1, STAT 3, STAT 4, STAT 5, STAT 6, CREB, Lyn, p-S6, Cbl, NF-kB, GSK3β, CARMA/Bcl10 and Tcl-1.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### EXAMPLES

### Example 1: Analysis of AML Patients

Patient Samples:

Sets of fresh or cryopreserved samples from patients can be analyzed. The sets can consist of peripheral blood mononuclear cell (PBMC) samples or bone marrow mononuclear cell (BMMC) samples derived from the blood of AML patients. All patients will be asked for consent for the collection and use of their samples for institutional review board (IRB)-approved research purposes. All clinical data is de-identified in compliance with Health Insurance Portability and Accountability Act (HIPAA) regulations. Sample inclusion criteria can require collection at a time point prior to initiation of induction chemotherapy, AML classification by the French-American-British (FAB) criteria as M0 through M7 (excluding M3), and availability of appropriate clinical annotations (e.g., disease response after one or two cycles of induction chemotherapy). Induction chemotherapy can consist of at least one cycle of standard cytarabine-based induction therapy (i.e., daunorubicin 60 mg/m2×3 days, cytarabine 100-200 mg/m2 continuous infusion×7 days); responses are measured after one cycle of induction therapy. Standard clinical and laboratory criteria can be used for defining complete responders (CR) in the patient samples. Leukemia samples obtained from patients who do not meet the criteria for CR or samples obtained from those who died during induction therapy are considered non-complete response (NR) for the primary analyses.

Cell Network Profiling Assays:

Cell network profiling assays involved measuring the expression of protein levels and their post-translational modification by phosphorylation in different populations of cells at baseline and after perturbation with various modulators. The populations that can be analyzed include myeloid leukemic cells, B cells, T cells, dendritic cells, monocytes, macrophages, neutrophils, eosinophils, and basophils. Other cells such as epithelial cells can also be analyzed.

A pathway “node” is defined as a combination of a specific proteomic readout in the presence or absence of a specific modulator. Levels of signaling proteins, as well as expression of cell surface markers (including cell lineage markers, membrane receptors and drug transporters), are detected by multiparameter flow cytometry using fluorochrome-conjugated antibodies to the target proteins. Multiple nodes (including surface receptors and transporters), using multiple modulators can be assessed in the two studies.

A minimum yield of 100,000 viable cells and 500 cells per gated sample in gate of interest can be used for each patient sample to be classified as evaluable.

Cyropreserved samples are thawed at 37° C., washed, and centrifuged in PBS, 10% FBS, and 2 mM EDTA. The cells are resuspended, filtered, and are washed in RPMI cell culture media, 1% FBS, then are stained with Live/Dead Fixable Aqua Viability Dye (Invitrogen, Carlsbad, Calif.) to distinguish non-viable cells. The viable cells are resuspended in RPMI, 1% FBS, aliquoted to 100,000 cells/condition, and are rested for 1-2 hours at 37° C. prior to cell-based functional assays or staining for phenotypic markers. Each condition can include 2 to 5 phenotypic markers (e.g., CD45, CD33), up to 3 intracellular stains, or up to 3 additional surface markers.

Cells are incubated with modulators, at 37° C. for 3-15 minutes, then fixed with 1.6% paraformaldehyde (final concentration) for 10 minutes at 37° C., pelleted, and permeabilized with 100% ice-cold methanol and stored at −20° C. For functional apoptosis assays, cells are incubated for 24 hours with cytotoxic drugs (i.e. Etoposide or Ara-C and daunorubicin), then re-stained with Live/Dead Fixable Aqua Viability Dye to distinguish non-viable cells before fixation and permeabilization, washed with FACS Buffer (PBS, 0.5% BSA, 0.05% NaN3), pelleted, and stained with fluorescent dye-conjugated antibodies (Becton Dickenson-Pharmingen, San Diego, Calif.) to both surface antigens (CD33, CD45) and the signaling protein targets.

Data Acquisition and Cytometry Analysis:

Data is acquired using FACS DIVA software on both LSR II and CANTO II Flow Cytometers (BD). For all analyses, dead cells and debris are excluded by FSC (forward scatter), SSC (side scatter), and Amine Aqua Viability Dye measurement. Leukemic cells are identified as cells that lacked the characteristics of mature lymphocytes (CD45++, CD33−), and that fit the CD45 and CD33 versus right-angle light-scatter characteristics consistent with myeloid leukemia cells. Other cell populations are identified using markers known in the art.

### Statistical Analysis and Stratifying Node Selection

a) Metrics:

The median fluorescence intensity (MFI) is computed for each node from the intensity levels for the cells in the gate of interest. The MFI values are then used to compute a variety of metrics by comparing them to the various baseline or background values, i.e. the unstimulated condition, autofluorescence, and isotype control. The following metricscan be computed in these studies: (1) Basal MFI=log 2(MFIUnmodulated Stained)−log 2(MFIGated Unstained (Autofluoresence)), designed to measure the basal levels of a certain protein under unmodulated conditions; (2) Fold Change MFI=log 2(MFIModulated Stained)−log 2(MFIUnmodulated Stained), a measure of the change in the activation state of a protein under modulated conditions; (3) Total Phospho MFI=log 2(MFIModulated Stained)−log 2(MFIGated Unstained (Autofluorescence)), a measure of the total levels of a protein under modulated conditions; (4) Fold over Control MFI=log 2(MFIStain)−log 2(MFIControl), a measure of the levels of surface marker staining relative to control antibody staining; (5) Percent Cell Positivity=a measure of the frequency of cells that have surface markers staining at an intensity level greater than the 95th percentile for control antibody staining

An additional metric is designed to measure the levels of cellular apoptosis in response to cytotoxic drugs: (6) Quadrant=a measure of the percentage of cells expressing high levels of apoptosis molecules (e.g. cleaved PARP and low levels of p-Chk2)

A low signaling node is defined as a node having a fold change metric or total phosphoprotein signal equal to I log 2(Fold) I>0.15. However, it is not necessary to use this as an exclusion criterion in this study.

b) Reproducibility Analysis

Two or more cryopreserved vials or fresh samples for each evaluable patient sample are obtained. All the vials are processed separately to access the assay reproducibility. Pearson and Spearman rank correlations were computed for each node/metric combination between the two data sets.

c) Univariate Analysis

All cell population/node/metric combinations are analyzed and compared across samples for their ability to distinguish between CR and NR samples. For each cell population/node/metric combination student t-test and Wilcoxon test p-Values are computed. In addition, the area under the receiver operator characteristic (ROC) (Hanley and McNeil, Radiology, 1982, Hanley and McNeil, Radiology, 1983, Bewick, et al, Critical Care, 2004) curve is also computed to access the diagnostic accuracy of each node for a given metric. The sensitivity (proportion of patients for whom a CR is correctly identified) and specificity (proportion of patients for whom a NR is correctly identified) data are plotted as ROC curves. A random result would produce an AUC value of 0.5. A (bio)marker with 100% specificity and selectivity would result in an AUC of 1.0. The cell population/node/metric combinations are independently tested for differences between patient samples whose response to standard induction therapy was CR vs NR. No corrections are applied to the p-values to correct for multiple testing. Instead, simulations are performed by randomly permuting the clinical variable to estimate the number of cell population/node/metric combinations that might appear to be significant by chance. For each permutation, nine donors are randomly chosen (without replacement) and assigned to the CR category and the remaining are assigned to the NR category. By comparing each cell population/node/metric combination to the permuted clinical variable, the student t-test p-values are computed. This process is repeated. The results from these simulations are then used to estimate the number of cell population/node/metric combinations that are expected to be significant by chance at the various p-values and compared with the empirical p-values for the number of cell population/node/metric combinations that were found to be significant from the real data.

The statistical analyses can be performed with the statistical software package R, version 2.7.0

d) Correlations Between Node:

Correlations between all pairs of cell population/node/metric combinations are accessed by computing Pearson and Spearman rank correlation.

e) Combinations of Nodes

Nodes that can potentially complement each other in combination to improve the accuracy of prediction of response to therapy are also explored. With a small size of the data set, a straightforward “corner classifier” approach for picking combinations can be adopted. Combinations that seem promising are also tested for their stability via a bootstrapping approach described below.

The corners classifier is a rules-based algorithm for dividing subjects into two classes (in this case the dichotomized response to induction therapy) using one or more numeric variables (defined in our study as a node/metric combination). This method works by setting a threshold on each variable, and then combining the resulting intervals (e.g., X<10, or Y>50) with the conjunction (and) operator (reference). This creates a rectangular region that is expected to hold most members of the class previously identified as the target (in this study CR or NR samples). Threshold values are chosen by minimizing an error criterion based on the logit-transformed misclassification rate within each class. The method assumes only that the two classes (i.e. response or lack of response to induction therapy) tend to have different locations along the variables used, and is invariant under monotone transformations of those variables.

A bagging, also known as bootstrapped aggregation, is used i to internally cross-validate the results of the above statistical model. Bootstrap re-samples are drawn from the original data. Each classifier, i.e. combination of cell population/node/metric, is fit to the resample, and then used to predict the class membership of those patients who were excluded from the resample. After repeating the re-sampling operation sufficiently, each patient acquires a list of predicted class memberships based on classifiers that are fit using other patients. Each patient's list is reduced to the fraction of target class predictions; members of the target class should have fractions near 1, unlike members of the other class. The set of such fractions, along with the patient's true class membership, is used to create an ROC curve and to calculate its AUC.

### Example 2: Analysis of Rheumatoid Arthritis Patients

Patient Samples:

Sets of fresh or cryopreserved samples from patients can be analyzed. The sets can consist of cells samples derived from the lymph nodes, synovium and/or synovial fluid of rheumatoid patients. All patients will be asked for consent for the collection and use of their samples for institutional review board (IRB)-approved research purposes. All clinical data is de-identified in compliance with Health Insurance Portability and Accountability Act (HIPAA) regulations.

Sample inclusion criteria can include: (i) A diagnosis of rheumatoid arthritis by the 1987 ACR criteria, (ii) Definite bony erosions, (iii) Age of disease onset greater than 18 years. (iv) Patient does not have psoriasis, inflammatory bowel disease, or systemic lupus erythematosus.

Standard clinical and laboratory criteria can be used for defining RA patients that are able to respond to a treatment in the patient samples. RA samples obtained from patients who do not meet the criteria for patients that are able to respond are considered non-complete responders for the primary analyses. Examples of possible treatments include nonsteroidal antiinflammatory drugs (NSAIDs) such as Acetylsalicylate (aspirin), naproxen (Naprosyn), ibuprofen (Advil, Medipren, Motrin), and etodolac (Lodine); Corticosteroid; Hydroxychloroquine; Sulfasalazine (Azulfidine); Gold salts such as Gold thioglucose (Solganal), gold thiomalate (Myochrysine), and auranofin (Ridaura); D-penicillamine (Depen, Cuprimine); Immunosuppressive medicines such as methotrexate (Rheumatrex, Trexall), azathioprine (Imuran), cyclophosphamide (Cytoxan), chlorambucil (Leukeran), and cyclosporine (Sandimmune).

Populations of cells that can be analyzed using the methods described in Example 1 include B cells, T cells, dendritic cells, monocytes, macrophages, neutrophils, eosinophils, and basophils. Other cells such as mesechymal cells and epithelial cells can also be analyzed.

### Example 3: Cellular and Intracellular Network Characterization of Cytokine JAK/STAT Signaling in Whole Blood Across Multiple Healthy Individuals: Defining “Normal”

Aberrant JAK/STAT signaling in hematopoietic cells has shown to be involved in certain hematological and immune diseases; thus, the regulation of JAK/STAT signaling is an important research area. Signaling pathway- and cell type-specific responses to various cytokines in the immune system signaling network can elicit a wide range of biological outcomes due to the combinatorial use of a limited set of kinases and STAT proteins. Although advances have been made in uncovering the intracellular mechanisms relating to cytokine signaling, the biological outcome may vary depending on composition and activation state of the cellular network. Single Cell Network Profiling (SCNP) by flow cytometry allows the interrogation of intracellular signaling networks within a heterogeneous cellular network, such as in unfractionated whole blood. We applied SCNP to investigate cytokine-induced JAK/STAT signaling in whole blood across healthy human donors (n=l 1) to 1) measure the relative contribution of signaling across multiple cell subsets; 2) measure the kinetics of signaling activation and resolution across cytokines and cell subsets; 3) measure the variation among donors in their overall signaling characteristics. Our aim was to better characterize “normal” cytokine responses across healthy individuals as a basis to eventually describe abnormal states.

Method:

Whole blood from 11 healthy donors (20-65 yrs, 7 males, 4 females, 8 Caucasians, 2 Hispanics, 1 East Asian) was stimulated at 37° C. in 96-well plates with a low, medium, and high dose of GM-CSF, IFN-α, IL-27 and IL-6, each added separately, as described in Example 5. For each dose, a stimulation time course was run with 6 time points between 3 and 45 minutes. Each well had a final concentration of 90% whole blood. The SCNP assay was performed using a fluorophore-labeled antibody cocktail to simultaneously measure signaling in six distinct cell populations, including: neutrophils, CD20+ B cells, CD3+CD4+T cells, CD3+CD4− T cells (CD8 enriched), CD3−CD20− lymphocytes (NK cell enriched), and CD14+ monocytes. The median fluorescent intensity of phospho (p)-STAT1(Y701), p-STAT3(Y705), and p-STAT5(Y694) were measured in each defined cell population for each experimental condition.

Results:

This SCNP assay was relatively high-throughput and provided high-content data, that equates to 19,000 gel lanes if attempted by Western analysis (11 donors×4 cytokines×4 concentrations×6 time points×6 cell subsets×3 p-readouts). In general, each cytokine demonstrated unique dose-dependent signaling characteristics (e.g., activation/termination kinetics, magnitude of response) for each cell type analyzed, and in some cases, the kinetics differed between p-STAT readouts within the same cell subset for the same cytokine. For instance, IL-6 induced signaling was only observed in CD4+ T cells and monocytes with peak p-STAT3 levels at 3 minutes followed by p-STAT1 and p-STAT5 at 10-15 minutes. In addition, signal resolution fell to baseline levels at 45 minutes in monocytes, while the CD4+ T cells showed sustained elevated signaling, suggesting a cell-type specific regulation. In contrast to IL-6, IFN-α stimulation activated all 3 STAT proteins, peaking at 10 minutes with similar kinetics in all cell subsets. However, IFN-α signaling resolution was faster and almost complete at 45 minutes in monocytes, while in the all other subsets the signal was sustained. This efficient signal termination in monocytes was also observed with GM-CSF→p-STAT5, while neutrophils maintained persistent p-STAT5 levels. IL-27 induced p-STAT1 and p-STAT3 in T cell subsets, B cells, and monocytes with peak activation at 30 minutes. In general, signaling characteristics were remarkably uniform across the healthy donors. IL-6→p-STAT3 was particularly consistent across time points and ligand concentrations, while p-STAT1 and p-STAT5 showed more variation. More results are provided in Example 5.

Approaching cell signaling from the perspective of the cellular network under physiological conditions (whole blood) allows for a more comprehensive and clinically relevant view of the signaling state of complex tissues. As many JAK/STAT targeting small molecule compounds enter the clinic, this study provides an important reference point for comparison with signaling networks that have become altered either by the pathological disease state or by therapy.

### Example 4: Single Cell Network Profiling (SCNP) of IFN-A Signaling Pathways in Peripheral Blood Mononuclear Cells from Healthy Donors: Implications for Disease Characterization, Treatment Selection, and Drug Discovery

The antiviral and antitumor effects of IFN-α, have been exploited for the treatment of viral infections such as hepatitis C (HCV) as well as for various malignancies, such as hairy cell leukemia and melanoma. However, widespread use of IFN-α for these and other indications is severely hampered by significant side effects which can have a major impact on patient quality of life. Thus, a greater understanding of intracellular signaling pathways regulated by IFN-α may guide in the selection of patients whose disease will have an optimal response with tolerable side effects to this cytokine. Specifically, the Signal Transducer and Activation of Transcription (Stat) transcription factors are known to play a critical role in transducing IFN-α mediated signals. Single cell network profiling (SCNP) is a multiparameter flow-cytometry based approach that can be used to simultaneously measure extracellular surface makers and intracellular signaling proteins in individual cells in response to externally added modulators. Here, we use SCNP to interrogate IFN-a signaling pathways in multiple cell subsets within peripheral blood mononuclear cells (PBMCs) from healthy donors.

This study was designed to apply SCNP to generate a map of IFN-α-mediated signaling responses, with emphasis on Stat proteins, in PBMCs from healthy donors. The data provides a reference for future studies using PBMCs from patient samples in which IFN-α-mediated signaling is aberrantly regulated.

Methods:

IFN-α-mediated signaling responses were measured by SCNP in PBMC samples from 12 healthy donors. PBMCs were processed for flow cytometry by fixation and permeabilization followed by incubation with fluorochrome-conjugated antibodies that recognize extracellular lineage markers and intracellular signaling molecules. The levels of several phospho-proteins (p-Stat1, p-Stat3, p-Stat4, p-Stat5, p-Stat6, and p-p38) were measured in multiple cell populations (CD14+ monocytes, CD20+ B cells, CD4+ CD3+ T cells, and CD4− CD3+ T cells) at 15 minutes, 1, 2 and 4 hours post IFN-α exposure as described in Example 6.

Results:

The data revealed distinct phospho-protein activation patterns in different cell subsets within PBMCs in response to IFN-α exposure. For example, activation of p-Stat4 was detected in T cell subsets (both CD4+ and CD4− T cells), but not in monocytes or B cells. Such cell-type specific activation patterns likely play a key role in mediating specific functions within different cell types in response to IFN-α. Differences in the kinetics of activation by IFN-α for different phospho-proteins were also observed. The peak response for activation of p-Stat1, p-Stat3, and p-Stat5 was at 15 minutes in most of the cell types interrogated in this study, whereas for the activation of p-Stat4, p-Stat6, and p-p38 it was at 1 hr in the majority of cell types tested. The relationships between phospho-protein readouts in each cell subset were determined by calculating the Pearson correlation coefficients. For example, the activation of p-Stat1 and p-Stat5 at 15 minutes was positively correlated in both B cells and T cells. More results are provided in Example 6.

The activation of intracellular signaling proteins was measured with emphasis on Stat transcription factors in PBMC subsets from healthy donors. We have analyzed the relationships between the activation states of phospho-proteins in the IFN-α signaling network. Characterization of IFN-α signaling pathways in samples from healthy donors has provided a network map that can be used as a reference for identifying alterations in IFN-α signaling that are the consequence of disease and/or therapeutic intervention. Future studies using SCNP to characterize IFN-α signaling pathways in PBMCs from patients with diseases such as viral infections or cancer may enable the optimization of IFN-α dosing and the identification of patient stratification biomarkers as well as the discovery of novel therapeutic agents.

### Example 5: Normal Cell Response to Erythropoietin (EPO) and Granulocyte Colony Stimulating Factor (G-CSF)

Normal cell signaling response to EPO and G-CSF was characterized through comparison to signaling response observed in samples from a subclass of patients with myelodysplatic syndrome (MDS) referred to herein as “low risk” patients. 15 samples of healthy BMMCs (from patients with no known diagnosis of disease) and 14 samples of BMMCs from patients who belonged to a subclass of patients with myelodysplastic syndrome were used to characterize normal cell response. The 14 samples of low risk patients were obtained from MD Anderson Cancer Center in Texas. The low risk patients were diagnosed as per standard of care at MD Anderson Cancer Center. The 15 samples of healthy BMMCs were obtained through Williamson Medical Center and from a commercial source (AllCells, Emeryville, Calif.). The samples obtained through Williamson Medical Center were collected with informed consent from patients undergoing surgeries such as knee or hip replacements.

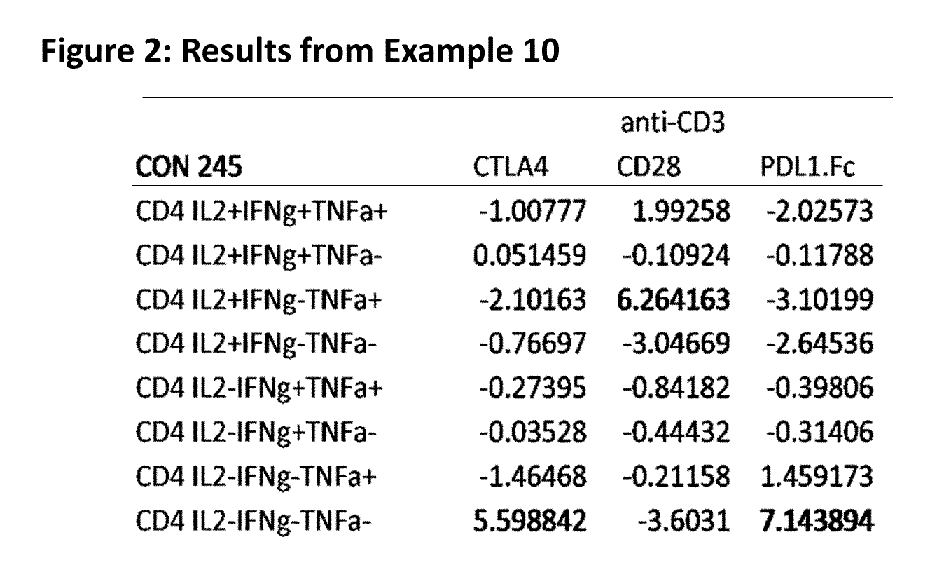

Each of the normal and the low risk samples were separated in aliquots. The aliquots were treated with a 3 IU/ml concentration of Erythropoietin, a 50 ng/ml concentration of G-CSF and both a 3 IU/ml concentration of Erythropoietin and a 50 ng/ml concentration of G-CSF. Activation levels of pStat1, pStat3 and pStat5 were measured using flow cytometry at 15 minutes after treatment with the modulators. In addition to the Stat proteins measured, several other elements were measured in order to separate the cells into discrete populations according to cell type. These markers included CD45, CD34, CD71 and CD235ab. CD45 was used to segregate Lymphocytes, Myeloid(p1) cells and nRBCs. The nRBCs were further segregated into 4 distinct cell populations based on expression of CD71 and CD235ab: m1, m2, m3 and m4. These cell populations correspond to RBC maturity and are illustrated in FIG. 2.

Distinct signaling responses were observed in the different discrete cell populations. FIG. 2 of U.S. Ser. No. 12/877,998 illustrates the different activation levels of pStat1, pStat3 and pStat5 observed in EPO, G-CSF and EPO+G-CSF treated Lymphocytes, nRBC1 cells, Myeloid(p1) cells and stem cells. Activation levels observed in different samples from the normal and low risk populations are plotted as dots. As shown in FIG. 2, different cell discrete populations demonstrated different induced activation levels. Although this was true in both the healthy and the low risk patients, the different discrete cell populations exhibited a narrower range of induced activation levels in then normal samples than in the low risk samples. These observations accord with the common understanding that diseased cells exhibit a wider range of different signaling phenotypes than normal cells.

Additionally, cell differentiation in disease may be inhibited or stunted, causing cells to exhibit characteristics that are different from other cells of the same type.

### Example 6: Normal Cell Response to Varying Concentrations of GM-CSF, IL-27, IFNα and IL-6

Kinetic response to varying concentrations of modulators was investigated in normal samples (i.e. samples from persons who have no diagnosis of disease). 11 normal samples were donated with informed consent by Nodality Inc. employees and processed at Nodality Inc. in South San Francisco, Calif. The samples were treated with 4 different modulators (GM-CSF, IL-27, IFNα and IL-6) at 4 different concentrations of the modulator and activation levels of pStat1, pStat3 and pStat5 were measured at different time points. Activation levels were measured at 3, 5, 10, 15, 30 and 45 minutes using flow cytometry-based single cell network profiling. The concentrations of the stimulators are tabulated below:

TABLE 3
Stimulator Concentrations

| | low | | med | | hi | |
|---|---|---|---|---|---|---|
| GM-CSF | 0.1 | ng/ml | 1 | ng/ml | 10 | ng/ml |
| IL-27 | 1 | ng/ml | 10 | ng/ml | 100 | ng/ml |
| IFNa | 1000 | IU | 4000 | IU | 100000 | IU |
| IL-6 | 1 | ng/ml | 10 | ng/ml | 100 | ng/ml |

Activation levels of different cell surface markers were also profiled using single cell network profiling and used in conjunction with gating to segregate the cells into discrete cell populations. In the gating analysis, SSC-A and FSC-A were first used to segregate lymphocytes from non-lymphocytes. CD14 and CD4 were then used to segregate the non-lymphocytes into populations of neutrophils and CD 14+ cells (monocytes). CD3 and CD20 were then used to segregate the lymphocytes into populations of CD20+ (B Cells), CD3+ (T Cells) and CD20−CD3− cells. CD4 was used to segregate the CD3+ T cells into populations of CD3+CD4− and CD3+CD4+ T cells.

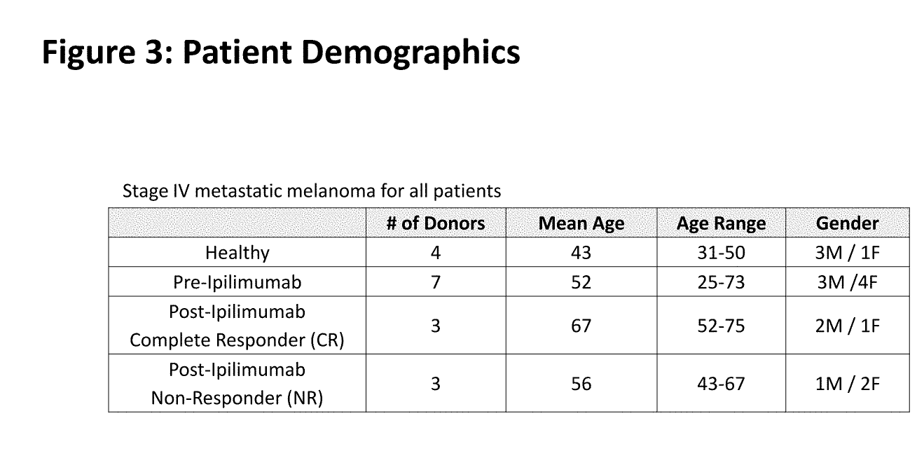

FIG. 3 of U.S. Ser. No. 12/877,998 illustrates the kinetic responses of different discrete cell populations in the normal samples. The line graphs contained in FIG. 3 of U.S. Ser. No. 12/877,998 plot the activation levels observed in all of the donors over the time intervals at which they were measured. The different concentrations of IL-6 tabulated above are represented by solid and dashed lines. Generally, the normal samples demonstrated similar activation profiles over time according to the concentration of sample given. Different concentrations of the modulator IL-6 yielded dramatically different activation profiles for some of the Stat phosphoproteins measured. For example, IL-6-induced pStat3 response varied at early time points (5-15 minutes) for the different concentrations of IL-6 but became more uniform at later time points. This uniformity of response supports the idea that normal cells exhibit a narrow range of activation.

Different discrete cell populations demonstrated unique responses to modulation. The neutrophils exhibited very low IL-6 induced activation as compared to the CD4+ T cells and monocytes. Between the CD4+ T cells and monocytes, several differences in activation profiles were observed. Monocytes showed a peak activation of IL-6-induced pStat1 activity at a different time point than the CD4+ T cells. Although both the monocytes and the CD4+ T cells demonstrated a drop-off in pStat3 activity after 15 minutes, the drop-off was much more dramatic in the monocytes. The difference in the slopes is illustrated in FIG. 3 of U.S. Ser. No. 12/877,998 by the use of boxes. This observation confirms the utility of using additional metrics which describe the dynamic response such as ‘slope’ and liner equations to represent dynamic response to induced activation.

### Example 7: Study Examining Modulated Proteomic Readouts in Pre-Treatment Peripheral Blood Mononuclear Cells (PBMC) from Patients with Metastatic Melanoma Who Received Ipilimumab

The current example identified signaling differences in PBMC samples from patients with metastatic melanoma vs. healthy donors. The study assessed using SCNP technology to identify, in pre-treatment cryopreserved PBMC samples from patients with metastatic melanoma, differential immune signaling between ipilimumab responsive and non-responsive patients.

Metastatic melanoma accounts for approximately 52,000 deaths annually worldwide. The incidence of melanoma in men is increasing at a faster rate than any other malignancy and in women is second only to lung cancer. The median survival of melanoma patients with distant metastases is less than one year. In phase 3 controlled trials conducted to date in metastatic melanoma, only one therapy, ipilimumab, has shown a survival benefit.

Ipilimumab is a fully human monoclonal antibody that blocks cytotoxic T-lymphocyte-associated antigen 4 (CTLA-4)-mediated T-cell suppression thus enabling potentiation of the antitumor T-cell response. In a randomized, controlled phase 3 trial in patients with previously treated metastatic melanoma, the hazard ratio for death with ipilimumab alone compared to gp100 vaccine alone was 0.66, (p=0.003). The beneficial effect of ipilimumab on overall survival was independent of age, sex, baseline lactate dehydrogenase level, metastatic disease stage, and receipt or nonreceipt of previous interleukin-2 therapy.

Ipilimumab benefits only a subset of patients, and has the potential to cause severe adverse effects that are mechanism-associated, including colitis; thus, there is need for biomarkers predictive of drug response to enable the identification of patients who will and will not benefit from this therapeutic.

The current Example gives the experimental design used to obtain results described in subsequent Examples. The purposes were:
1) to compare immune cell subset representation in cryopreserved pre-treatment PBMC samples from patients with melanoma and cryopreserved PBMC samples from healthy donors,
2) to compare basal and modulated signaling responses in cryopreserved pre-treatment PBMC samples from patients with melanoma and cryopreserved PBMC samples from healthy donors.
3) to describe functional distinctions between samples from ipilimumab responsive and non-responsive patients, including immune cell subset representation, basal and modulated signaling responses, and outcome of 48 hour T cell activation using cryopreserved PBMCs collected pre-treatment,
4) to verify that samples from patients with melanoma who responded to ipilimumab treatment had altered responsiveness to cytokines compared to samples from healthy donors or to patients with melanoma who did not respond to ipilimumab. Nodes evaluated in all T cells and CTLA-4+ CD4 T cells include IL-6→p-STAT1 and p-STAT3, and IL-10→p-STAT1 and p-STAT3, IL-12→p-STAT4, IL-15→p-STAT5, and IL-21→p-STAT1 and p-STAT3
5) to verify that CTLA-4 expressing T cells (CD4+/CTLA-4+/Foxp3−) in samples from patients with melanoma who responded to ipilimumab treatment have reduced anti-CD3→p-CD3zeta and anti-CD3→p-ZAP70 response compared to CTLA-4 expressing T cells in samples from healthy donors or patients with melanoma who did not respond to ipilimumab, and
6) to verify using samples from patients with melanoma:
   i. That increased levels of CTLA-4 in CD4 and CD8 T cells were associated with response to ipilimumab
   ii. That increased levels of PD-1 in CD4 and CD8 T cells were associated with non-response to ipilimumab
   iii. That increased inhibition by antiCTLA-4 activating antibody of anti-CD3 induced ICOS and PD-1 expression on CD4 and CD8 T cells at 48 hours was associated with response to ipilimumab
   iv. That increased inhibition by anti CTLA-4 activating antibody of anti-CD3 induced decreases in frequencies of T-cells producing cytokines (TNFα, IL-2, IFNγ) at 48 hours was associated with response to ipilimumab. Samples from patients that respond to ipilimumab were expected to have fewer frequencies of polyfunctional T cells—defined as being positive for 2+ cytokines—when cultured with anti CTLA-4 activating antibody.
   v. Increased PD-L1.fc activity was associated with non-response to ipilimumab as measured by anti-CD3 stimulation+/−anti-PD 1 activating antibody.

### Sample Collection

PBMC samples were collected prospectively as part of the required study procedures for the compassionate use ipilimumab study conducted in Naples, Italy. Fresh 3 mL peripheral blood samples were collected in EDTA at the pre-treatment study visit and the PBMCs were cryopreserved at the site within 24 hours using the institutional cryopreservation procedure. The cryopreserved samples were shipped in batch using a liquid nitrogen cryoshipper to a California laboratory.

Assay Procedure

Upon thaw, cells underwent a Ficoll-Hypaque gradient purification after which the total cell number, leukocyte count and cell recovery were determined for each sample. The samples were split into 3 assay types: A) Live cell staining of phenotypic markers, B) short term signaling whereby the cells are incubated with modulators, fixed, and permeabilized, and C) Two day T cell activation with secondary modulators. The samples were incubated with a cocktail of fluorochrome-conjugated antibodies that recognize extracellular lineage markers and intracellular epitopes including phospho-epitopes within intracellular signaling molecules. To assess sample health, cells were stained in a separate well for cPARP, except in the TCR signaling analyses where cPARP was evaluated in every well.

### Assay Panel

Live Cell Phenotypic Staining. PBMCs were stained for T cell expression levels of the four CD28 (CTLA-4) family members and antigen presenting cell (APC) expression levels of three ligands (B7 family members). The aim of this analysis was determine the basal expression levels of CTLA-4, its family members, and its ligands.

Short term signaling. PBMCs was modulated and stained with antibody panels designed to distinguish multiple cell subsets (Table 4) within a given cell lineage. The aim of this analysis was to quantify the signaling potential both within T cells that express CTLA-4, as well as the other immune cells that T cells interact with.

Two day T cell activation. T cells were activated by CD3 cross-linking in the presence or absence of anti-CTLA-4 activating antibody or PD-1 ligand (PD-L1.Fc). The aim of this analysis was to assess CTLA-4 and PD-1 functionality by quantifying how CTLA-4 and PD-1 activation affects intracellular cytokine production and activation markers (ICOS and PD-1) expression.

TABLE 4
List of backbone stains:

| Panel | |
|---|---|
| | TCR Signaling |
| Markers | CD4, CD8, CD45RA, CTLA-4, Foxp3 |
| Subsets: | CD4 vs. CD8 T cells |
| | CD45RA+ naive vs CD45R− memory/effector |
| | CTLA-4+/Foxp3+ Tregs |
| | CTLA-4+ non-Tregs |
| | T cell signaling |
| Markers | CD3, CD4, CD45RA, CTLA-4 |
| Subsets: | CD3+/CD4+ T cells vs. CD3+/CD4− T cells |
| | CD45RA+ naive vs CD45R− memory/effector |
| | CTLA-4_hi vs. CTLA-4_low T cells |
| | B cell signaling |
| Markers | CD4, CD19, CD27, IgD |
| Subsets: | CD4 vs. CD19 to discriminate B cells |
| | CD27+ memory/effector vs CD27− naive B cells |
| | IgD+ vs IgD− class switched B cells |
| | APC signaling |
| Markers | CD11c, CD14, HLA-DR, CD3/CD19/CD56 dump |
| Subsets: | CD14+/HLA-DR+ monocytes |
| | CD14+/HLA-DR− myeloid derived suppressor cells |
| | CD14−/HLA-DR+/CD11c+ dendritic cells |
| | CD28 Family: T cell - Live and 48 hour activation |
| Markers | CD3, CD4, CD8 |
| Measure | CD28, CTLA-4, ICOS, PD-1 |
| Expression | |
| | B7 Family: APC - Live |
| Markers | CD11c, CD14, HLA-DR, CD3/CD19/CD56 dump |
| Measure | CD80, CD86, PD-L1 |
| Expression | |
| | Intracellular Cytokines - 48 hour activation |
| Markers | CD3, CD4, CD8 |
| Cytokines | TNFa, IFNγ, IL-2 |

TABLE 5
Nodes below are listed by biological category.

| Modulator | Readouts | Cell Type |
|---|---|---|
| CD3 Xlink | p_CD3zeta, p_ZAP70, | TCR Signaling |
| | p_S6, p_PLCγ2 | |
| IL-6 | p_STAT1/3/5 | T cell Signaling |
| IL-10 | p_STAT1/3/5 | T cell Signaling |
| IL-15 | p_STAT1/3/5 | T cell Signaling |
| IL-21 | p_STAT1/3/5 | T cell Signaling |
| IL-2 + IL-4 | p_STAT4/5/6 | T cell Signaling |
| IL-12 | p_STAT4/5/6 | T cell Signaling |
| IL-21 | | T cell Signaling |
| IFNα | p_STAT1/3/5 | T cell Signaling |
| IFNγ | p_STAT1/3/5 | T cell Signaling |
| IL-6 | p_STAT1/3/5 | B cell Signaling |
| IL-21 | p_STAT1/3/5 | B cell Signaling |
| CD40L | IkBa, p_ERK, p_AKT | B cell Signaling |
| anti-IgM | IkBa, p_ERK, p_AKT | B cell Signaling |
| R848 | IkBa, p_ERK, p_AKT | B cell Signaling |
| IFNα | p_STAT3/5/6 | APC Signaling |
| IL-4 | p_STAT3/5/6 | APC Signaling |
| GM-CSF | p_STAT3/5/6 | APC Signaling |
| LPS | IkBa, p_ERK, p_AKT | APC Signaling |
| None | ICOS, PD-1, CD28, CTLA-4 | T cell - Live stain |
| None | CD80, CD86, PD-L1 | APC - Live stain |
| aCTLA-4 (Act) | Intracellular TNFa, IL-2, | T cell - 48 hr activation |
| | IFNγ | |
| CD28 | Intracellular TNFa, IL-2, | T cell - 48 hr activation |
| | IFNγ | |
| PD-L1.Fc | Intracellular TNFa, IL-2, | T cell - 48 hr activation |
| | IFNγ | |
| aPD-1 (Block) | Intracellular TNFa, IL-2, | T cell - 48 hr activation |
| | IFNγ | |
| aCTLA-4 (Act) | ICOS, PD-1, CTLA-4, CD28 | T cell - 48 hr activation |
| CD28 | ICOS, PD-1, CTLA-4, CD28 | T cell - 48 hr activation |
| PD-L1.Fc | ICOS, PD-1, CTLA-4, CD28 | T cell - 48 hr activation |
| aPD-1 (Block) | ICOS, PD-1, CTLA-4, CD28 | T cell - 48 hr activation |

### Example 8

Methods:

10 melanoma pre-treatment samples (4 from pts with stable disease, 5 with progressive disease, and 1 non-assessable) and 3 healthy samples were examined by a process as generally described in Example 7. SCNP analysis of cytokine and TCR signaling nodes focused on the CD4+ T cell subsets defined by intracellular staining of CTLA-4 and Foxp3. Metrics included Equivalent number of Reference Fluorophores (ERF; median fluorescence intensity calibrated per plate), and Uu (the proportion of cells responding relative to basal activity).

Results:

Lymphocyte viability was >90% in healthy donors, but only 7/10 melanoma met the >60% viability cut-off for analysis. Treg frequencies did not differ between healthy and melanoma samples. Anti-CD3 induced p-CD3-zeta in CD4+ T cells (Table 4). CD4+ T cells from melanoma patients had reduced Uu and ERF compared to CD4+ T cells from healthy subjects. Within CD4+ T cells, melanoma samples signaled highest in the Treg cell subset while signaling magnitude in healthy donor cells was greatest CTLA-4− T cells. Due to low sample numbers, comparison of signaling between responders and non-responders was not feasible.

Conclusions:

Signal transduction activities differed between CTLA-4 defined CD4+ subsets, and between healthy and melanoma samples.

TABLE 6
Anti-CD3 → p-CD3-zeta signaling in CD4+ T cell subsets

| | | CTLA-4− | | CTLA-4+ |
|---|---|---|---|---|
| Sample | Metric | Foxp3− | CTLA-4+ Foxp3− | Foxp3+ (Treg) |
| Healthy | ERF | 7877 ± 507 | 6410 ± 937 | 6142 ± 653 |
| Melanoma | ERF | 2011 ± 1332 | 1855 ± 1587 | 3604 ± 1885 |
| Healthy | Uu | 0.92 ± 0.02 | 0.82 ± 0.04 | 0.97 ± 0.01 |
| Melanoma | Uu | 0.66 ± 0.08 | 0.59 ± 0.07 | 0.78 ± 0.10 |

### Example 9

The following is an example of a method to assess the signaling capacity of antigen presenting cells in retrograde signaling and to interrupt the mechanism which suppresses the activation of T cells.

It is know that antigen presenting cells (APCs) and T cells interact to activate the T cells through the CD3 receptor on the T cell and through CD80 and CD86 on the APCs and CD28 on the T cells. For example, Treg cells can condition APCs through a mechanism dependent on interactions between CTLA-4 and CD80 and CD86 to express indoleamine 2,3-dioxygenase (IDO), which is a potent regulatory molecule that induces the catabolism of tryptophan into proapoptotic metabolites that result in the suppression of activation of effector T cells. IDO induction was found to depend on high expression of CTLA-4 on the Treg cells. The present example analyzes the role of cell signaling in the APCs extrinsic mechanism.

Take APCs from a patient suffering from melanoma. Use a process similar to that shown in Example 1 to detect activatable elements in the APCs. For example, contact the APCs with a modulator that can stimulate the CD80 and CD86 receptors in the same manner as CTLA4 to induce signaling in the APCs for the production of IDO and metabolites that suppress activation of T cells. An example of this reagent is a compound that comprises a portion of CTLA4 and an antibody, called CTLA4.fc. It is available from suppliers such as R+D Systems (Minneapolis, Minn.), Cell Networks (Canton Mass.), and Life Technologies (Carlsbad, Calif.). In some cases, the APCs may need to pre-activated with (such as with LPS or IFNa) to induce upregulation of CD80 and CD86 expression. Correlate cell activation at 15 minutes with IDO expression at 4 hours.

Detect signaling molecules in the APCs using the appropriate binding elements specific for the signaling molecules, for example measure all tyrosine phosphorylation to detect any modulation. Use the permeabilization and staining procedure similar to that outlined above in Example 1.

Once the cell signaling information is obtained for these pathways in the APCs, select an agent(s) that can block the pathway involved in the production of IDO to stop the dampening of T cell activation.

### Example 10

T cells and APCs also react through the PD-1 receptor on T cells and its ligand on the APC, PD-L1, to inhibit the activation of T cells. It is analogous to the CTLA4/CD 80 and CD 86 interaction as it controls T cell activation. The following example shows the effect of cell to cell communication on T cells using some of the molecules present in the T cell/APC interaction. The results show what cytokines normal cells are making and that this production can be affected using antibodies.

Previously cryopreserved PBMC cells from normal healthy individuals were taken and contacted with modulators, such anti-CD3 antibodies along with one of the following: antiCTLA4, anti CD28, and PDL1.fc. The modulators were kept in contact with the cells for 48 hours and then production of IL-2, TNFa, and IFNg was measured. PD-L 1.fc was obtained from R+D Systems, CTLA4 was obtained from BD, San Jose, Calif. and CD28 from BD, San Jose.

The subpopulation of CD4 cells were measured for intracellular cytokine production (IL-2, TNFa, and IFNg) using a LSR II from BD, San Jose, Calif.

The results are shown in FIG. 2. The three columns of results show the increase or decrease in the frequency of CD4 cells with the cytokines which were treated with anti-CD3 plus either anti CTLA4, anti CD28, or PD-L1.fc compared to CD4 cells treated with anti-CD3 only. The results show that overall, the use of the modulators caused a decrease in cytokine production over baseline in many instances. The results also show that cell to cell crosstalk is important in immune response and can be the basis for one embodiment of the present invention.

### Example 11

Background:

CTLA-4 is an immune regulator expressed by regulatory (Tregs) and activated Tcells. Ipilimumab, an anti-CTLA-4 monoclonal antibody, is approved for treatment of unresectable/metastatic melanoma. However treatment is expensive, benefits only a subset of patients and has been associated with significant adverse effects. Biomarkers are needed to identify patients most likely to respond. Single cell network profiling (SCNP) is a multiparametric flow cytometry-based assay that quantitatively measures both phenotypic markers and changes in intracellular signaling proteins in response to modulation, enabling analysis of cell signaling networks.

Objectives:

Functional proteomic profiling of immune signaling pathways in PBMC subsets in healthy donors and patients with metastatic melanoma receiving ipilumumab treatment.

Methods:

3 healthy and 13 melanoma patient cryopreserved samples, including 7 collected pre-treatment, and 6 collected post-treatment, were analyzed by SCNP. FIG. 3 shows subject demographics. Cells were assessed for viability, cell subset frequencies and signaling in response to different modulators. A node is defined as combination of modulator and intracellular readout (e.g. IL-6→p-STAT3). Cytokine and TCR- or FcγR-induced activation of STAT 1, 3, 5, CD3ζ and Erk was evaluated in multiple immune cell populations and comparisons made based on disease, treatment time point and clinical responses.

Results: Compared to healthy donors, melanoma patient monocytes displayed lower induction of IL-10→p-STAT1, IL-10→p-STAT3, IL-6→p-STAT1, IL-6→p-STAT3 and FcγR→p-Erk. Similarly, TCR→p-CD3ζ was reduced in the CD45RA− but not CD45RA+ CD4+ and CD4− cell subsets. These data suggest a generalized hyporesponsivity of circulating immune cell subsets in both the innate and adaptive arms of the immune response in melanoma patients. Of note, the same observations pertained to samples collected from melanoma patients regardless of in vivo administration of ipilumumab and independently from clinical response to treatment. Specific effects of ipilimumab treatment on T cell signaling were also observed compared to healthy donors and pre-treatment melanoma samples. Post-treatment samples had diminished TCR→p-Erk, TCR→p-CD3z in CD45RA+ but not CD45RA− CD4+ and CD4− subsets and IL-15→p-STAT5 in CD4− T cells. None of these markers associated with clinical response to ipilumumab. Noteworthy, in samples from patients non-responsive to ipilumumab the percentage of circulating Treg (CD25+, FOXP3+) were higher compared to those of complete responders and healthy donors. Basal p-Erk levels in CD45RA+CD4+ T cells were also elevated in this population.

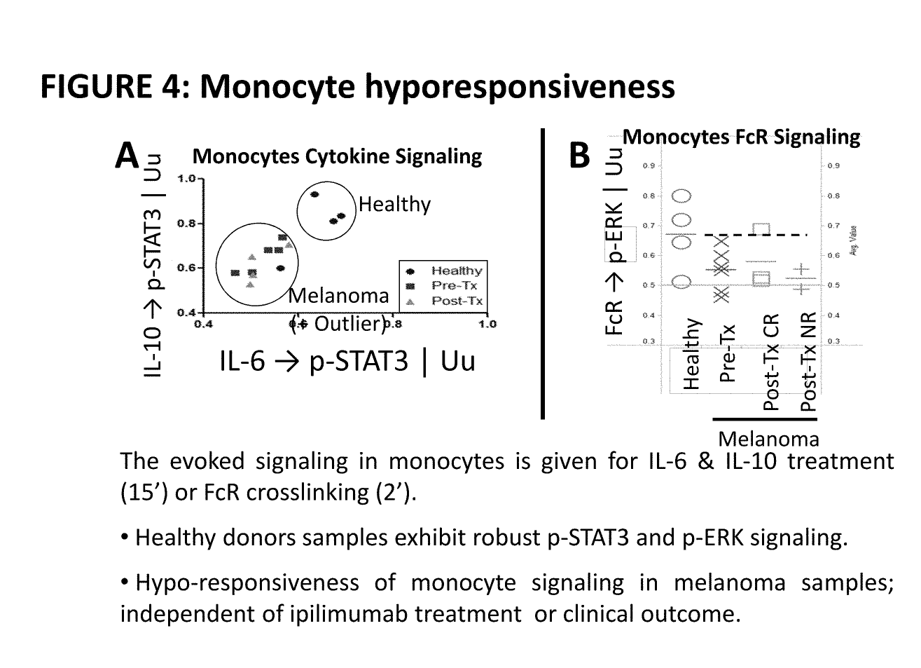

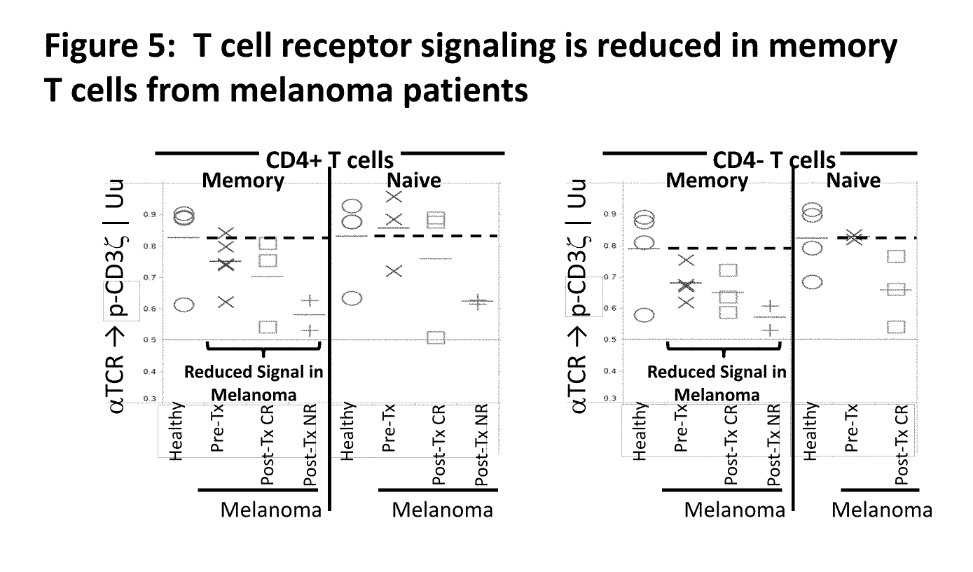

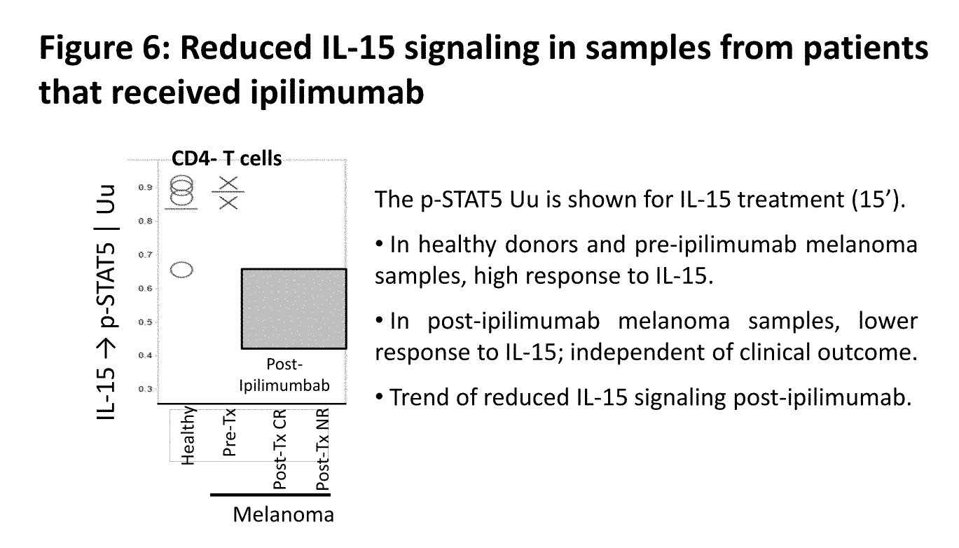

Monocytes of melanoma patients showed hyporesponsiveness compared to monocytes of healthy donors. Healthy donor samples exhibited robust p-STAT3 and p-ERK signaling in response to IL-6, IL-10, or FcR, whereas samples from all melanoma patients showed reduced signaling, regardless of ipilimumab treatment or clinical outcome. See FIG. 4. T cell receptor signaling is reduced in memory T cells from melanoma patients. See FIG. 5. There was reduced responsiveness to IL-15 (p-STAT5) in samples from patients that received ipilimumab. In healthy donors and in patients pre-ipilimumab, there was high responsiveness to IL-15, whereas in post-ipilimumab samples, the response to IL-15 was lower, regardless of clinical outcome. See FIG. 6.

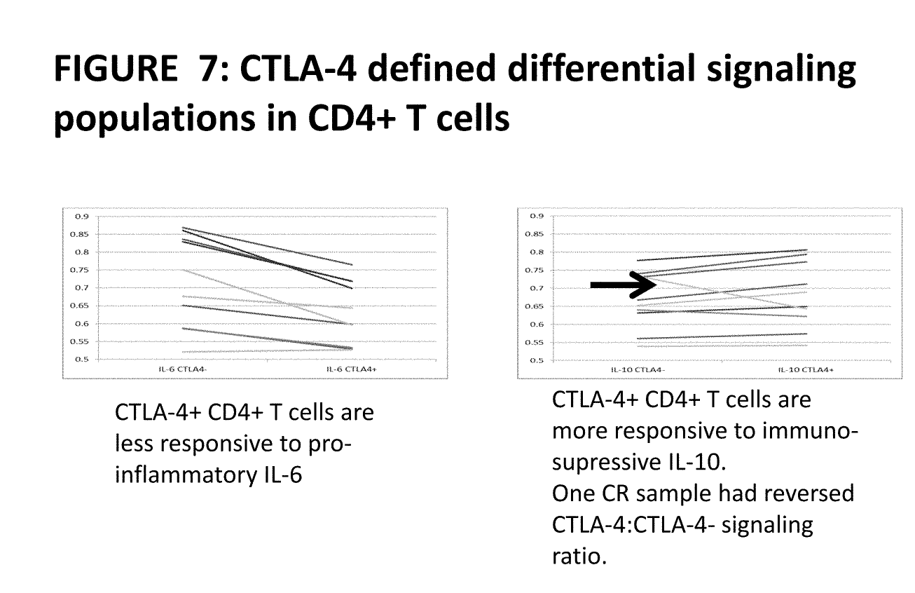

CTLA-4 defined differential signaling populations in CD4+ T cells. See FIG. 7. Finally, ipilimumab promotes in vitro T cell activation.

In sum, 1) melanoma samples display a trend of hypo-responsiveness compared to healthy samples, independent of ipilimumab treatment or clinical outcome. Specifically, melanoma samples displayed decreased cytokine→p-STAT3 signaling in monocytes, decreased FcγR signaling in monocytes, and decreased TCR signaling in memory T cells. 2) Ipilimumab-treated patient samples showed reduced IL-15→p-STAT5 signaling relative to healthy and untreated donor samples. This has potential implications for side effects and combination therapies. SCNP analysis of PBMC from healthy donors and melanoma patients pre- and post-ipilimumab treatment identified immune signaling differences between melanoma and healthy and between pre- and post ipilimumab treatment. This Example demonstrates that SCNP can be used to distinguish signaling abnormalities in specific populations of cells from melanoma patients, some of which have implications for both side effect prediction and combination therapies.

### Example 12

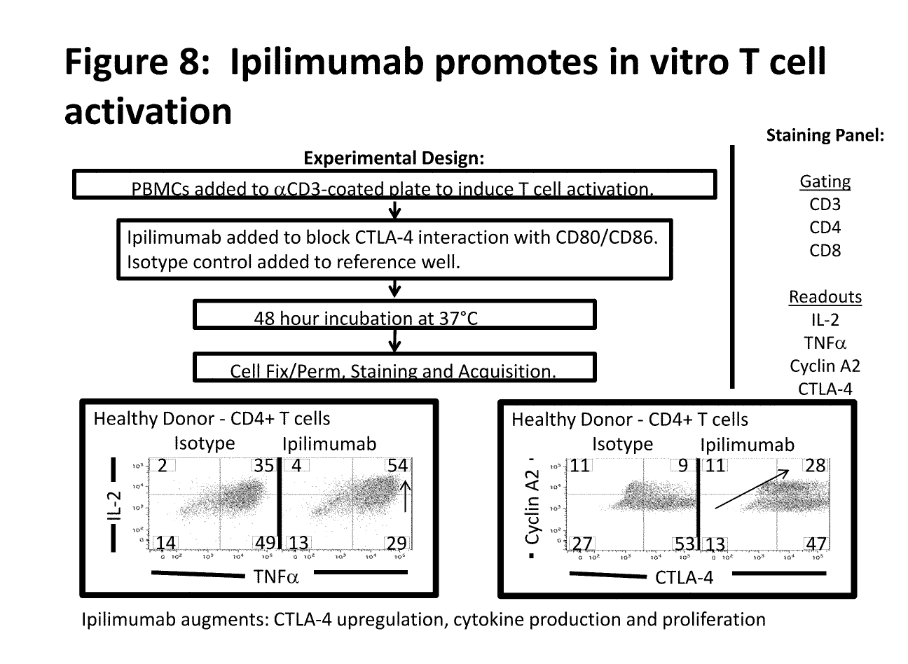

In this example, PBMCs were added to a αCD3-coated plate to induce T cell activation, then ipilimumab was added to block CTLA-4 interaction with CD80/86. Isotype control was added to a reference well. The cells were incubated at 37° C. for 48 hr, then fixed, permeabilized, stained, and data acquired as described in the Examples above. Cells were gated by CD3, CD4, and CD8, and the readouts were IL-2, TNF-α, Cyclin A2, and CTLA-4. Results are shown in FIG. 8. Ipilimumab augmented CTLA-4 upregulation, cytokine production, and proliferation.

### Example 13

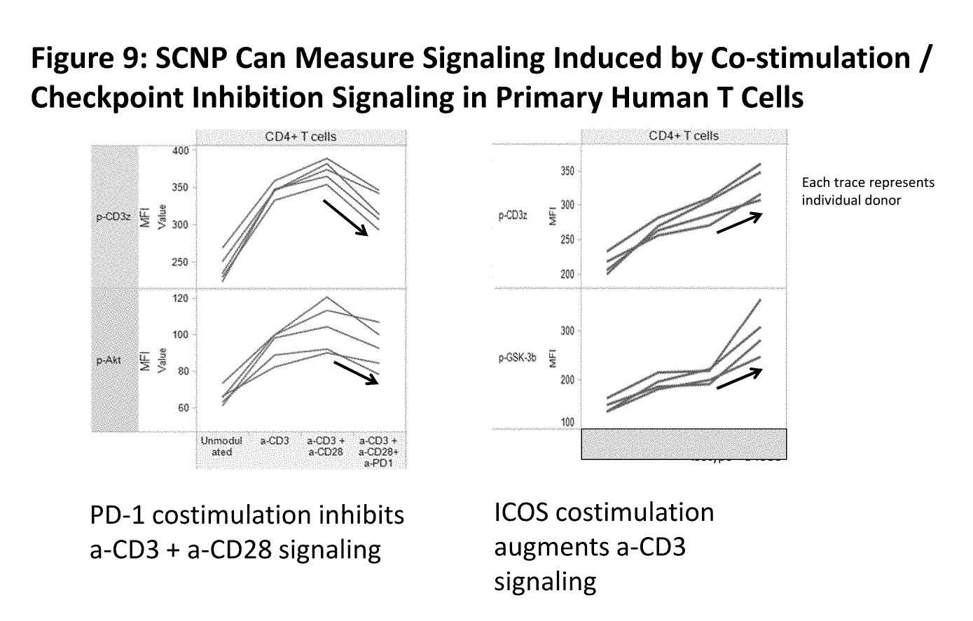

CTLA-4 (the target of Ipilimumab) and PD-1 both provide checkpoint inhibition of T cell activation that immunotherapy seeks to circumvent. In this Example it is shown that signaling inhibition through PD-1 crosslinking can be measured. Knowing if a sample shows strong PD-1 inhibition may be used to help inform if a patient should take anti-PD-1 or anti-CTLA-4 (e.g., ipilimumab). In this example, T cells were treated with a variety of modulators and response determined. In one experiment, T cells were unmodulated, modulated only with anti-CD3, modulated with both anti-CD3 and anti-CD28, using biotin-avidin crosslinking, and modulated with anti-CD3 and anti-CD28 with crosslinking and also with anti-PD1. p-AKT or p-CD3z were the activatable elements detected in the TCR activation pathway as indicators of TCR activation. The crosslinking was achieved by constructing biotinylated anti-CD3 and anti-CD28 antibodies, contacting the cells with the antibodies, then adding avidin so that the antibodies crosslinked into complexes. See FIG. 9, left panel. The greater response to the CD3/CD28 combination illustrates the positive co-stimulation effect of these two, and the decrease in that response with addition of anti-PD 1 illustrates the negative co-stimulation effect of PD 1. In a further experiment, T cells were unmodulated, modulated with anti-CD3 (also with isotype control), and modulated with anti-CD3 and anti-ICOS, with biotinylated-avidin crosslinking. The anti-CD3 antibody is a mouse antibody while the anti-ICOS is hamster, so they could not be crosslinked using secondary antibodies. Activation of the TCR activation pathway was assessed by measuring levels of p-CD3z or p-GSK3b. See FIG. 9, right panel. The greater response with the addition of anti-ICOS indicates the positive co-stimulatory effect of ICOS.

This Example illustrates that SCNP can be used to determine changes, and magnitude of changes, in T-cell signaling induced by various agents, when occurring with activation of immunomodulatory receptors.

### Example 14

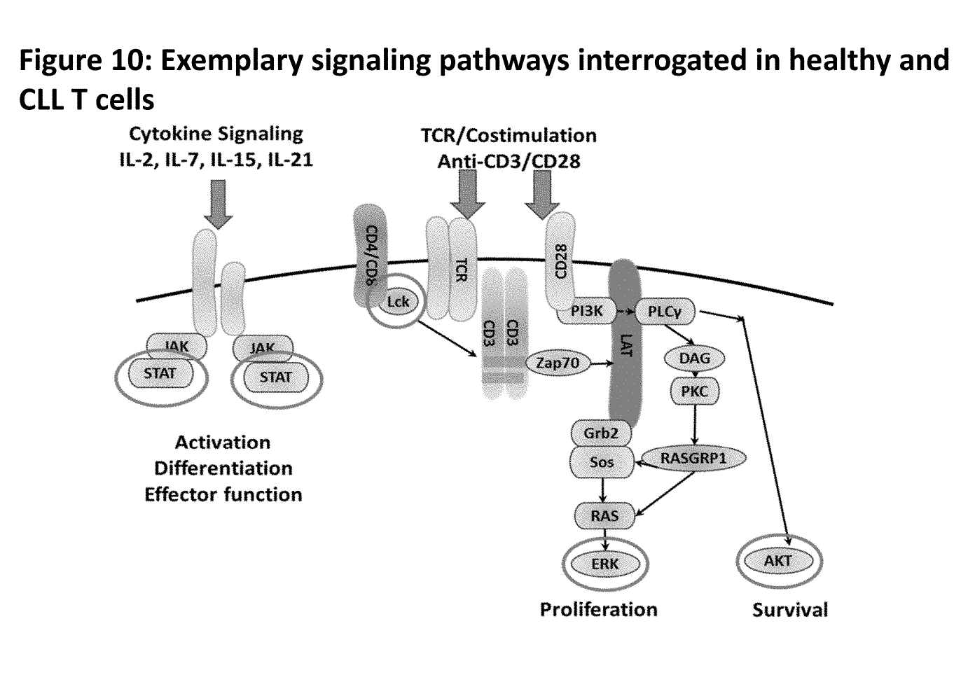

Inhibition of PD-1 signaling has shown clinical efficacy in the treatment of cancer. Profiling signaling networks of PD-1+ T cells can reveal biological alterations in disease with potential to yield disease-specific prognostic/predictive biomarkers and rationale for combining certain therapies. Single Cell Network Profiling (SCNP) was utilized to map signaling networks simultaneously in multiple T cell subsets in PBMC from chronic lymphocytic leukemia (CLL) patients and healthy donors (HD) (FIG. 10). Patient and Healthy donor characteristics are shown in Table 7, below:

TABLE 7

| Patient/Disease | | | |
|---|---|---|---|
| Characteristics | Subgroups | CLL Patients | Healthy Donors |
| Age | Min, Max | 53, 82 | 59, 78 |
| | Median | 65 | 66 |
| Gender | Male | 8 | 10 |
| | Female | 4 | 2 |

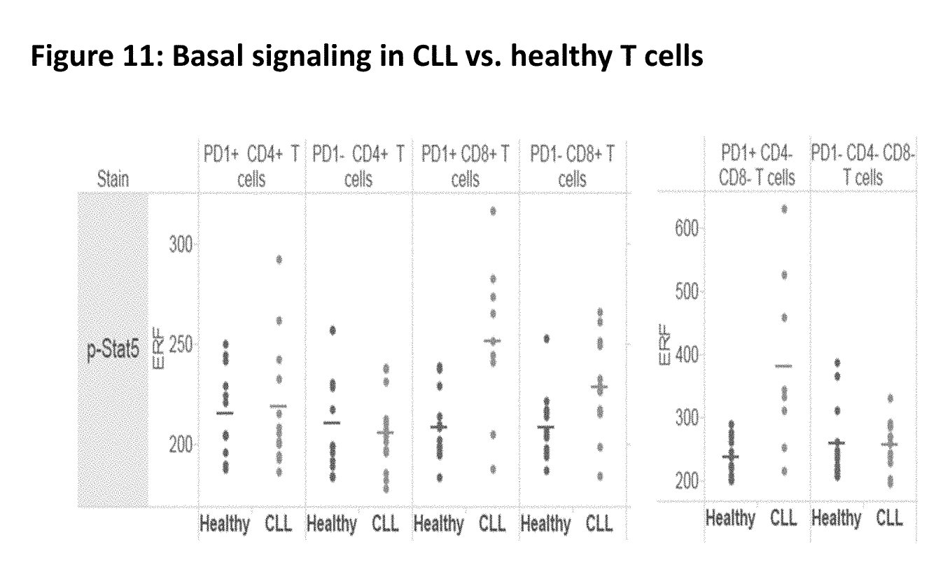

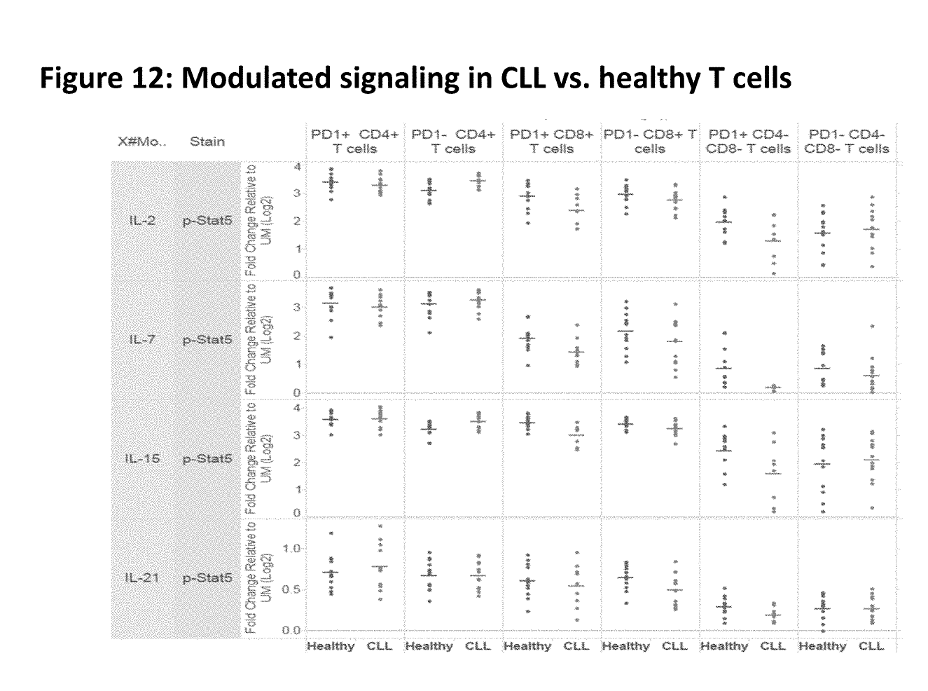

Overall CLL and HD did not differ significantly in PD-1 expression, although CLL samples were more heterogeneous, and significant differences were detected in basal and evoked signaling, particularly in PD-1+ CD8 T cells. Specifically, basal p-STAT5 was elevated in PD-1+ and PD-1− CD8 T cells from CLL compared to HD. See FIG. 11. Activation with IL-2, IL-7, or IL-15 increased p-STAT5 levels in T cells, with distinct findings observed for each cytokine across PD-1+ cell types (FIG. 12). Whereas IL-2, IL-7, and IL-15 treatment of PD-1+ CD4 cells resulted in similar p-STAT5 levels in CLL and HD, treatment of PD-1+ CD8 T and PD-1+ CD4−CD8− T cells resulted in lower inducible p-STAT5 levels in CLL as compared to HD, indicating dysfunctional signaling through these common γ-chain cytokines in these subsets (FIG. 12). Greater heterogeneity in CLL vs. HD cytokine responses was observed, suggesting the potential for subgroup identification.

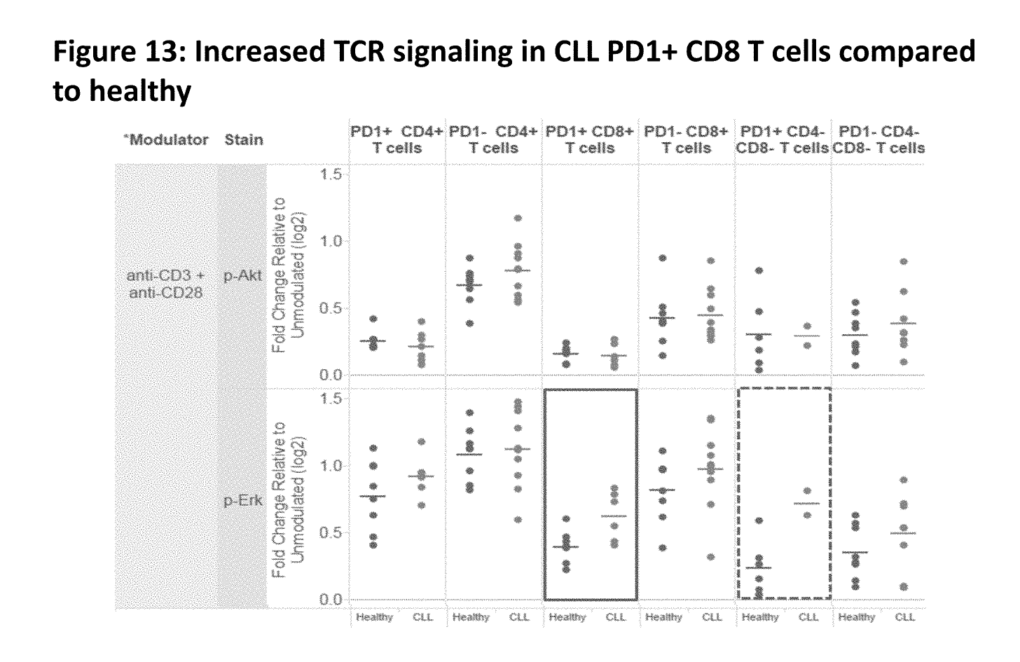

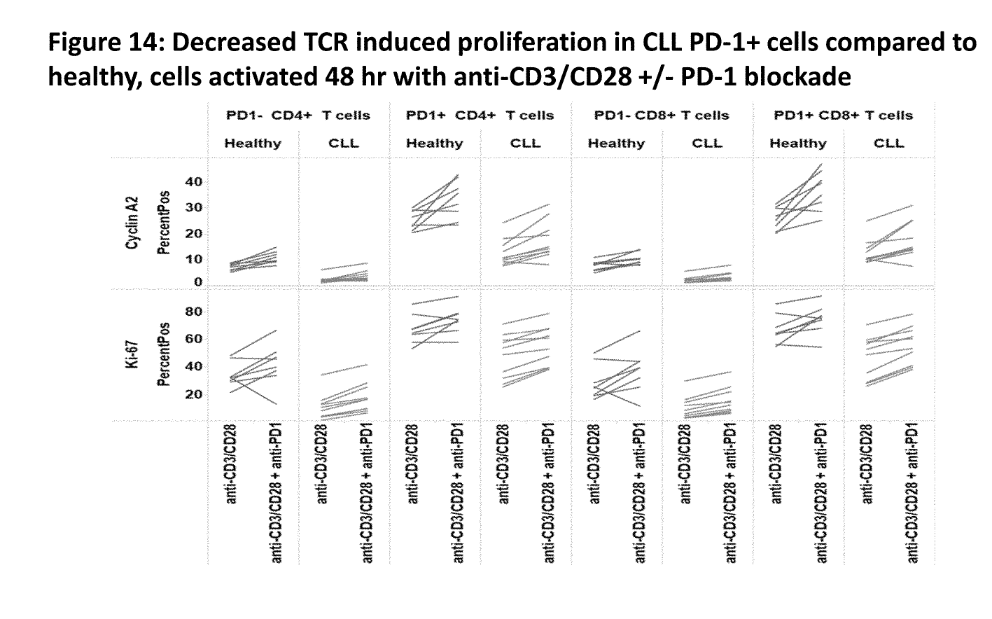

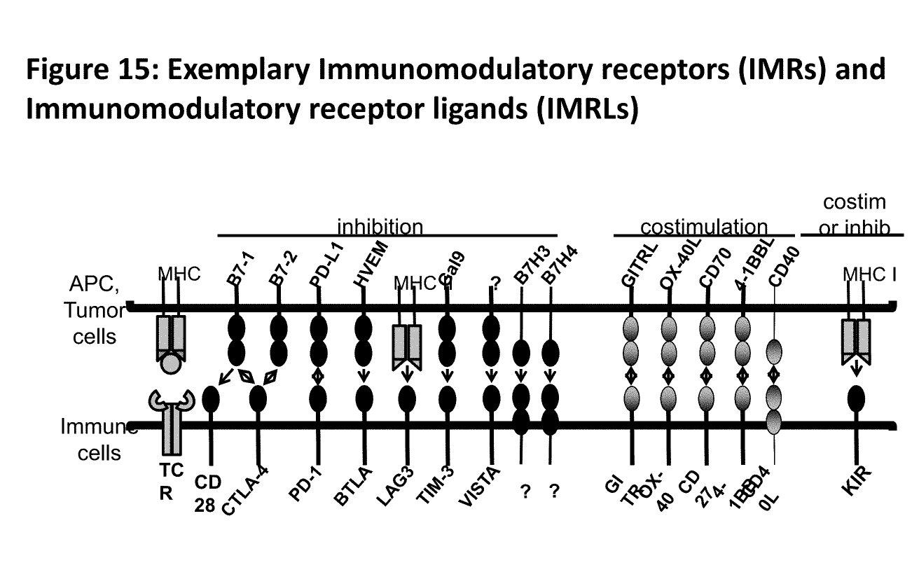

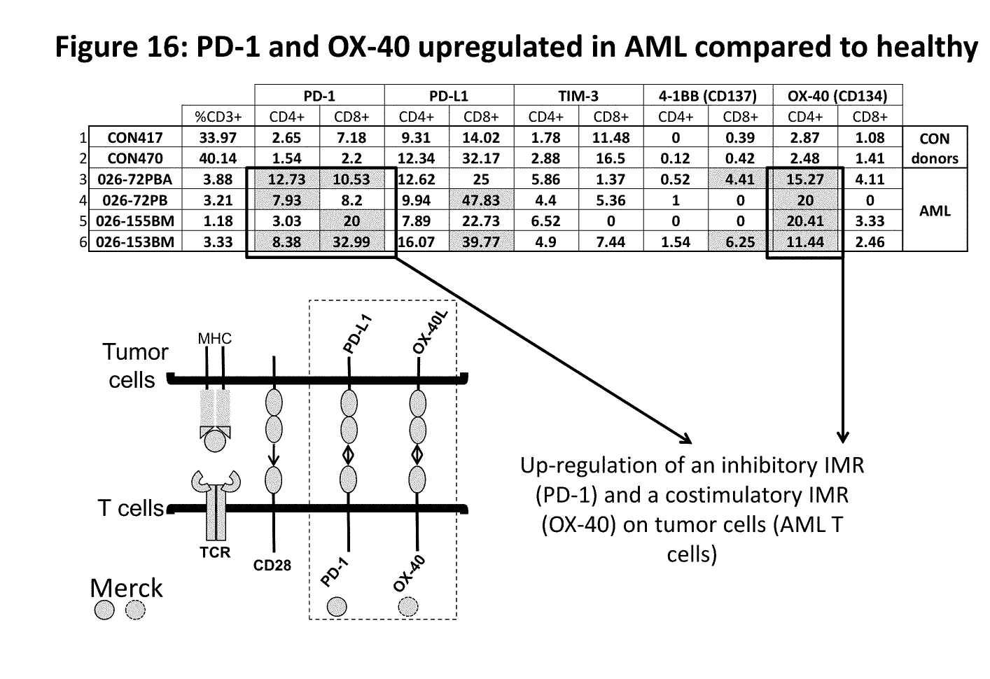

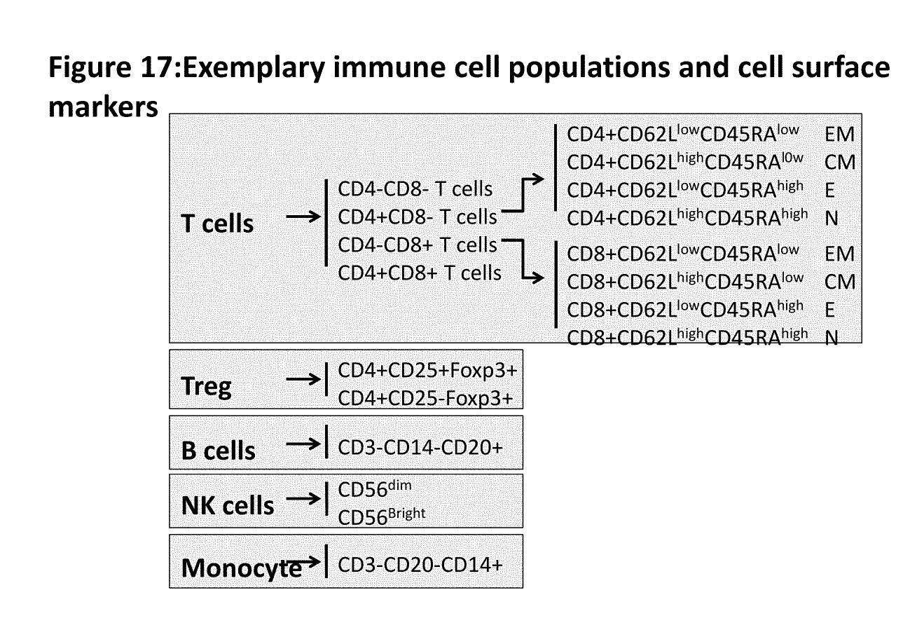

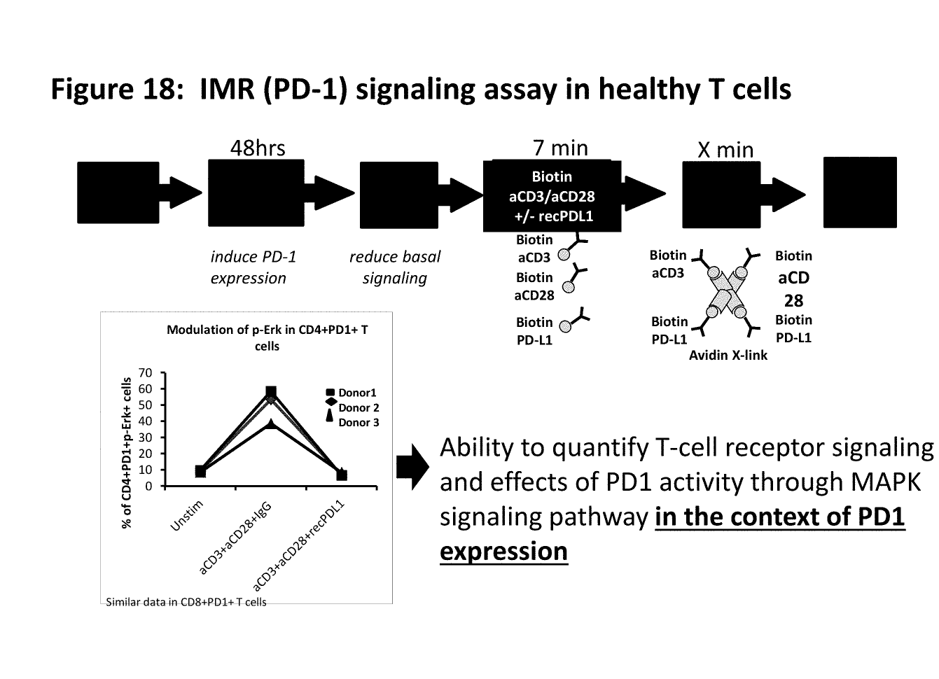

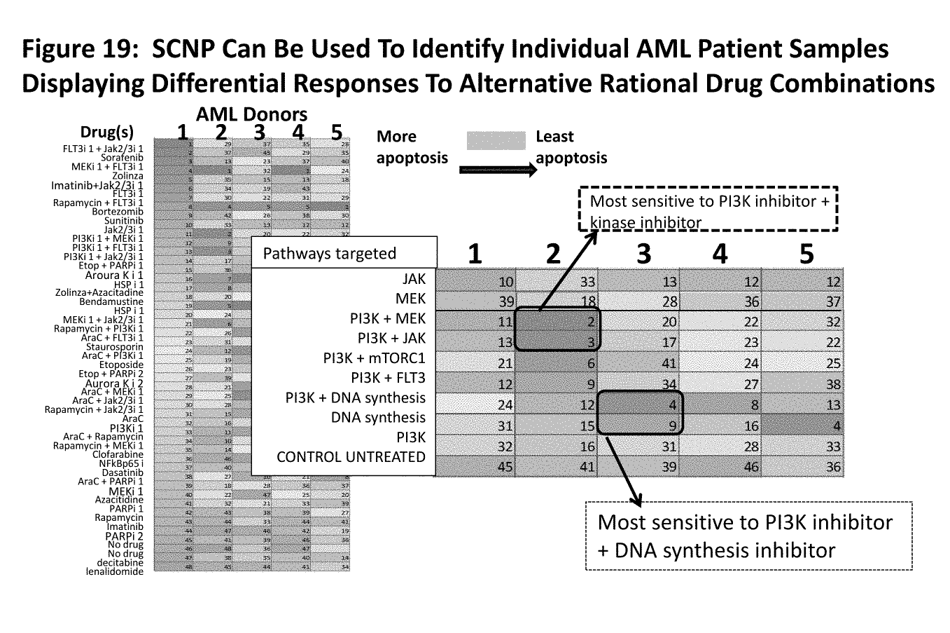

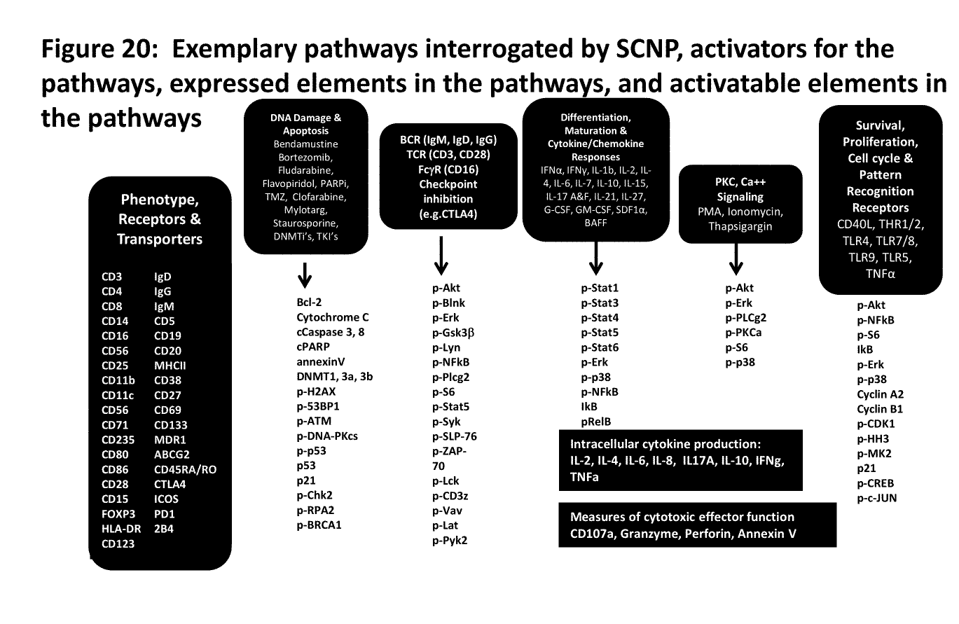

In contrast to reduced cytokine responsiveness, increased TCR modulated induction of p-Erk was observed in PD-1+ CD8 T cells in CLL vs HD (FIG. 13). Conversely, CLL T cells demonstrated decreased proliferation in response to CD3/CD28 stimulation, which could be partially reversed with PD-1 blockade (FIG. 14).

The dysregulated cytokine signaling, elevated TCR responsiveness and reduced proliferation observed in these cell subsets in CLL are consistent with the “pseudo-exhausted state” described in CLL.

Collectively, these data demonstrate the application of SCNP to interrogate signaling in PD-1+ T cells, the consequences of the tumor microenvironment on T cell exhaustion, and potential efficacy of therapeutics to restore T cell function.

### Example 15

Using the methods and compositions of the present disclosure we identified dysfunctional immune states in AML patients including the surface expression levels of important immunomodulatory receptors (IMRs) which are known to have a profound effect on immune function.

Briefly, using the methods and compositions provided herein we interrogated the expression of PD-1, PD-L1, LAG3, GITR, OX-40, 4-1BB, CD27, and TIM-3 across CD4+ and CD8+ T cell subsets isolated from peripheral blood mononuclear cells (PBMC) obtained from AML patients (4×) and healthy donor control patients (2×).

In the PBMC samples obtained from AML patients, we observed increased expression of OX-40 on the surface of CD4+ T cells relative to healthy control PBMC samples. See FIG. 16.

These results suggest that, OX-40 could be used as a pharmaceutical target to re-activate CD4 positive cells in AML patients using an OX-40 agonist. Further, these results suggest that OX-40 expression, along with the methods provided herein, could be used as a diagnostic test to select AML patients for OX-40 therapies comprising an OX-40 agonist or combination treatment therapies comprising an OX-40 agonist and other AML therapies.

This Example demonstrates
1) that the cell surface expression levels of a plurality of IMRs, both inhibitory and costimulatory, and the surface expression level of at least one IMRL, can be measured in single cells from a blood sample derivative (PBMC) taken from healthy individuals and individuals suffering from a pathological condition (cancer, in this case AML),
2) that a variety of cell populations may be interrogated for surface expression levels and gated based on a plurality of cell surface markers (e.g., CD3, CD4, CD8),
3) that altered surface expression of two of the IMRs (increased expression of both PD-1, an inhibitory IMR, and OX-40, a costimulatory IMR) can be detected in specific cell populations in specific sample types (increased surface expression levels of PD-1 in CD4+ and CD8+ cells from PBMC samples, compared to healthy controls, and increased surface expression levels of OX-40 in CD4+ cells in PBMC, compared to healthy controls),
4) that a trend toward altered (increased) surface expression of an IMRL (PD-L1) in PBMC in certain cells (CD8+ cells) in the AML patients helps corroborate the activation of the PD-1 pathway of immunosuppression.
5) that a trend toward altered (increased) expression of a second costimulatory IMR, 4-1BB, in two of the AML patients but not the other two, suggests that it may be useful in this cancer (AML) for classification or its modulation may be useful in monotherapy or combination therapy, or in this surface expression type of cancer (PD-1 increase, OX-40 increase, 4-1BB increase in two of the 4 AML patients but not the other two, suggestive that a surface expression classification of the cancer may be more useful in classifying the cancer, and/or in designing and implementing an immunotherapy, especially a combination immunotherapy, than traditional classifications of cancer)
6) that the altered surface expression levels suggest monotherapy (directed at the PD1 pathway, e.g., inhibit, or directed at the CD40 pathway, e.g., activate) or a combination therapy (both directed at the PD1 pathway, e.g., inhibit, and the OX-40 pathway, e.g., activate) for the particular disease (AML, or PD 1+, OX40+ cancer) and/or for the particular expression pattern (high PD-1, high OX-40).

### Example 16

This Example demonstrates that PBMC cells from healthy individuals may be induced to express IMRs and that IMR functional status can be assessed in the cells in which the IMR has been induced.

In brief, PBMCs were isolated from healthy blood donors according to approved protocol. PBMCs were first cultured in RPMI 1640 plus 10% FBS with anti CD3 and anti CD28 for 48 hours to increase levels of surface expression of PD-1. Subsequently, cells were washed and rested overnight in RPMI 1640 plus 10% FBS. For stimulation experiments, PBMC (1.5×10e5 cells) were incubated with anti-CD3, anti-CD28 and anti IgG Biotinylated Abs or anti-CD3, anti-CD28 biotinylated Abs plus biotinylated recombinant PDL1. Antibodies were then cross-linked with Avidin. At a certain time point, the reaction was stopped and the cell fixed with PFA 2.4%. Cells were them processed for SCNP analysis for selected read-outs. The protocol and results are shown in FIG. 18. Unstimulated cells showed low levels of an intracellular activatable element, p-ERK. Cells activated with a TCR activator, in the absence of an IMR modulator, exhibited high levels of p-ERK. Cells activated with a TCR activator, in the presence of an IMR modulator, exhibited low levels of p-ERK. All cells were gated so that PD1+ cells were used.

This Example illustrates that IMRs can be induced in cells from healthy individuals, providing a cell population that can be used, e.g., in screening agents for their effects in immunotherapy, that the functional status of the IMR can be measured in such cells and that the expression levels of the IMR can be measured in such cells.

### Example 17

This Example demonstrates that SCNP can be used for identifying rational drug combinations in cancer patients, e.g., AML patients.

The SCNP methods and compositions described herein were used to interrogate a number of different pathways in the presence of numerous different therapeutic agents, which differentially affected different pathways, in 5 different AML patients. See FIG. 19. The results show that different AML patients exhibited sensitivities in different pathways, as exhibited by increased levels of activatable elements in the apoptosis pathway for these patients (FIG. 19).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a patient suffering from cancer comprising treating the patient with a treatment;
wherein an aspect of treating the patient with the treatment is based on an outcome of a treatment decision process, wherein the treatment decision process comprises consideration of at least two of a first, second, and/or third quantitative value, or a value or values derived from the at least two quantitative values; and
wherein the first, second, and third quantitative values are obtained from results of a first, second, and/or third assay, respectively, wherein
(a) the first assay comprises determining surface expression levels of a first immunomodulatory receptor (IMR) of a first cell population cell population (CP) in a first sample from the patient;
(b) the second assay comprises determining functional status of a second IMR in single cells of a second CP or a subpopulation thereof in a second sample from the patient; and
(c) the third assay comprises determining surface expression levels of an IMR ligand (IMRL) for a third IMR in a third cell population in a third sample from the patient.

2. (canceled)

3. The method of claim 1 wherein the surface expression levels of the first IMR in first assay are determined in single cells, or the surface expression levels of the IMRL of the third IMR in the third assay are determined in single cells, or both and wherein the first, second and third assays are performed by flow cytometry or mass cytometry.

4-7. (canceled)

8. The method of claim 1 wherein the aspect of treating the patient comprises a decision to treat the patient or not treat the patient with the treatment, a choice of the treatment or a component of the treatment, a choice of the timing of the treatment or of a component of the treatment, a choice of a dosage of the treatment or a component of the treatment, or a combination thereof.

9. The method of claim 1 wherein the outcome of the treatment decision process comprises a first likelihood of the patient responding to the treatment, a second likelihood of prolongation of the patient's life due to receiving the treatment, or a third likelihood of the patient experiencing an adverse treatment effect, or any combination of the first, second, and/or third likelihoods.

10.-11. (canceled)

12. The method of claim 1 wherein the treatment is a combination treatment comprising an immunotherapy treatment, in a) and b) a plurality of IMRs is assayed, and wherein the aspect of the treatment comprises choice of the combination therapy.

13.-23. (canceled)

24. The method of claim 1 wherein the first and second samples comprise tumor-infiltrating lymphocytes (TILS) derived from a solid tumor sample and wherein the third sample comprises tumor cells derived from the same solid tumor sample.

25. (canceled)

26. The method of claim 1 wherein in c) a plurality of IMRLs is determined in single cells.

27. The method of claim 26 wherein the plurality of IMRLs comprises a plurality of IMRLS of FIG. 15 and the description thereof.

28. The method of claim 1 wherein in a) and b) a plurality of IMRs is assayed.

29. The method of claim 28 wherein the plurality of IMRs comprises a plurality of IMRs of FIG. 15 and the description thereof.

30. (canceled)

31. The method of claim 1 wherein the assay of the functional status of the IMR of b) comprises determining a change in an activation level of an intracellular activatable element or change in expression level of an intracellular expression element, wherein the activatable element is an activatable element of TABLE 1, or FIG. 20.

32.-35. (canceled)

36. The method of claim 1 wherein the first and second cell populations comprise a first and second immune cell population of TABLE 1 or FIG. 17.

37. (canceled)

38. The method of claim 36 wherein the cell populations are identified by surface expression levels of at least three of the cell surface markers of Table 1 or FIG. 17.

39.-41. (canceled)

42. A kit comprising
(i) a distinguishably detectable binding element configured for use in binding to and distinguishably detecting a first intracellular element, wherein a change in the expression level and/or activation level of the first intracellular element in a cell of an immune cell type in response to exposure of the cell to an activator of the immune cell type is indicative of activation of the cell; and
(ii) a distinguishably detectable binding element configured for use in binding to and distinguishably detecting a cell surface IMR on the cell or a cell surface IMRL on a cell of a population of cells of a non-immune cell type, and, optionally,
(iii) the activator.

43. (canceled)

44. The kit of claim 42 comprising a plurality of distinguishably detectable binding elements configured for use in binding to and distinguishably detecting a plurality of different cell surface IMRs or a plurality of different cell surface IMRLs.

45. The kit of claim 42 further comprising instructions for use of the kit in an assay for predicting the response of a patient to immunotherapy, wherein the immunotherapy is an immunotherapy that directly or indirectly affects activation of the population of cells of the immune cell type.

46. The kit of claim 42 further comprising a plurality of distinguishably detectable binding elements, each configured for use in binding to and distinguishably detecting a different cell surface marker, wherein the level of at least two of the plurality of different cell surface markers can be used to type the cell as a cell of an immune cell population, wherein the plurality of cell surface markers comprise a plurality of cell surface markers listed in TABLE 1 or FIG. 17.

47. (canceled)

48. The kit of claim 42 wherein the cell surface IMR or the cell surface IMRL comprises an IMR or an IMRL of FIG. 15 and the description thereof.

49. The kit of claim 42 wherein the IMR is PD-1 and the IMRL is PDL-1 or PDL-2.

50. The kit of claim 44 wherein the plurality of different surface IMRs and/or the plurality of different cell surface IMRLs are a plurality of different surface IMRs and/or a plurality of different cell surface IMRLs of FIG. 15 and the description thereof.

51. The kit of claim 50 wherein the plurality of IMRs comprise PD-1 and CTLA-4 and the plurality of IMRLs comprise at least two of B7-1, B7-2, PDL-1, and PDL-2.

52. (canceled)

53. The kit of claim 42 wherein the intracellular element is an intracellular activatable element and wherein the activatable element is an activatable element of TABLE 1 or FIG. 20.

54.-69. (canceled)

70. A method for screening a first agent at a first screening level comprising
(i) contacting a first immune cell population expressing a first IMR on their surfaces with the first agent and activating cells of the first population by contacting them with an activator;
(ii) activating cells of a second immune cell population expressing the first IMR on their surfaces that have not been contacted with the first agent by contacting them with the activator;
(iii) determining
(a) expression levels of an intracellular expression element in single cells of the first population or a subpopulation thereof and expression levels of the intracellular element in single cells of the second population or a subpopulation thereof, wherein the intracellular expression element is an element whose expression levels changes upon activation of the cells of the first and second immune cell populations, and/or
(b) activation levels of an intracellular activatable element in single cells of the first population or a subpopulation thereof and activation levels of the intracellular activatable element in single cells of the second population or a subpopulation thereof, wherein the intracellular activatable element is an activatable element whose activation level changes upon activation of the a cell of the first and second immune cell populations; and
(iv) making a determination to send or not send the agent to a second screening level based on the results of (iii).

71.-107. (canceled)

108. A method of treating a patient suffering from a pathological condition comprising
treating the patient with a treatment for the condition, wherein an aspect of treating the patient with the treatment is based on an outcome of a treatment decision process comprising consideration of a quantitative value, or a value or values derived from the quantitative value, wherein the quantitative value is obtained from results of an assay comprising determining functional status of an IMR in single cells of a immune cell population or a subpopulation thereof in a sample from the patient.

109. The method of claim 108 further comprising determining surface expression levels of the IMR in the single cells.

110. The method of claim 109 wherein the method is performed using a subpopulation of the immune cell population, and wherein single cells of the subpopulation are gated into the subpopulation on the basis of the surface expression level of the IMR of the single cell.

111. (canceled)

112. The method of claim 108 the determination of the functional status comprises performing flow cytometry or mass cytometry.

113-117. (canceled)
